(19) United States
(12) Patent Application Publication
(10) Pub. No.: US 2003/0203862 A1
(43) Pub. Date: Oct. 30, 2003

(54) Antisense modulation of MDM2 expression

(76) Inventors: Loren J. Miraglia, Encinitas, CA (US); Pamela Nero, San Diego, CA (US); Mark J. Graham, San Clemente, CA (US); Brett P. Monia, Encinitas, CA (US); Erich Koller, Carlsbad, CA (US); Ming Yi Chiang, San Diego, CA (US); Muthiah Manoharan, Carlsbad, CA (US)

Correspondence Address: Licata & Tyrrell P.C., 66 E. Main Street, Marlton, NJ, 08053, US

(21) Appl. No.: 10/005,344

(22) Filed: Dec. 4, 2001

Related U.S. Application Data
(63) Continuation-in-part of application No. 09/752,983, filed on Jan. 2, 2001, now abandoned.
Application No. 09/752,983 is a continuation of application No. 09/280,805, filed on Mar. 26, 1999, now Pat. No. 6,184,212.
Application No. 09/280,805 is a continuation-in-part of application No. 09/048,810, filed on Mar. 26, 1998, now Pat. No. 6,238,921.

(51) Int. Cl. A61K 48/00; C07H 21/04

(52) U.S. Cl. 514/44; 536/23.2

(57) ABSTRACT

Compounds, compositions and methods are provided for inhibiting the expression of human mdm2. The compositions include antisense compounds targeted to nucleic acids encoding mdm2. Methods of using these oligonucleotides for inhibition of mdm2 expression and for treatment of diseases such as cancers associated with overexpression of mdm2 are provided.

This application is a continuation-in-part of U.S. patent application Ser. No. 09/752,983, filed Jan. 2, 2001, which is a continuation of U.S. patent application Ser. No. 09/280,805, filed Mar. 26, 1999, now issued as U.S. Pat. No. 6,184,212, which is a continuation in part of U.S. patent application Ser. No. 09/048,810 filed Mar. 26, 1998, now issued as U.S. Pat. No. 6,238,921.

### FIELD OF THE INVENTION

This invention relates to compositions and methods for modulating expression of the mdm2 gene, a naturally present cellular gene implicated in abnormal cell proliferation and tumor formation. This invention is also directed to methods for inhibiting hyperproliferation of cells; these methods can be used diagnostically or therapeutically. Furthermore, this invention is directed to treatment of conditions associated with expression of the mdm2 gene. This invention is also directed to novel oligonucleotide compounds useful in antisense, or as ribozymes or aptamers.

### BACKGROUND OF THE INVENTION

Inactivation of tumor suppressor genes leads to unregulated cell proliferation and is a cause of tumorigenesis. In many tumors, the tumor suppressors, p53 or Rb (retinoblastoma) are inactivated. This can occur either by mutations within these genes, or by overexpression of the mdm2 gene. The mdm2 protein physically associates with both p53 and Rb, inhibiting their function. The levels of mdm2 are maintained through a feedback loop mechanism with p53. Overexpression of mdm2 effectively inactivates p53 and promotes cell proliferation.

The role of p53 in apoptosis and tumorigenesis is well-known in the art (see, in general, Canman, C. E. and Kastan, M. B., Adv. Pharmacol., 1997, 41, 429-460). Mdm2 has been shown to regulate p53's apoptotic functions (Chen, J., et al., Mol. Cell Biol., 1996, 16, 2445-2452; Haupt, Y., et al., EMBO J., 1996, 15, 1596-1606). Overexpression of mdm2 protects tumor cells from p53-mediated apoptosis. Thus, mdm2 is an attractive target for cancers associated with altered p53 expression.

Amplification of the mdm2 gene is found in many human cancers, including soft tissue sarcomas, astrocytomas, glioblastomas, breast cancers and non-small cell lung carcinomas. In many blood cancers, overexpression of mdm2 can occur with a normal copy number. This has been attributed to enhanced translation of mdm2 mRNA, which is thought to be related to a distinct 5′-untranslated region (5′-UTR) which causes the transcript to be translated more efficiently than the normal mdm2 transcript. Landers et al., Cancer Res. 57, 3562, (1997).

Several approaches have been used to disrupt the interaction between p53 and mdm2. Small peptide inhibitors, screened from a phage display library, have been shown in ELISA assays to disrupt this interaction [Bottger et al., J. Mol. Biol., 269, 744 (1997)]. Microinjection of an anti-mdm2 antibody targeted to the p53-binding domain of mdm2 increased p53-dependent transcription [Blaydes et al., oncogene, 14, 1859 (1997)].

A vector-based antisense approach has been used to study the function of mdm2. Using a rhabdomyosarcoma model, Fiddler et al. [Mol. Cell Biol., 16, 5048 (1996)] demonstrated that amplified mdm2 inhibits the ability of MyoD to function as a transcription factor. Furthermore, expression of full-length antisense mdm2 from a cytomegalovirus promoter-containing vector restores muscle-specific gene expression.

Antisense oligonucleotides have also been useful in understanding the role of mdm2 in regulation of p53. An antisense oligonucleotide directed to the mdm2 start codon allowed cisplatin-induced p53-mediated apoptosis to occur in a cell line overexpressing mdm2 [Kondo et al., Oncogene, 10, 2001 (1995)]. The same oligonucleotide was found to inhibit the expression of P-glycoprotein [Kondo et al., Br. J. Cancer, 74, 1263 (1996)]. P-glycoprotein was shown to be induced by mdm2. Teoh et al [Blood, 90, 1982 (1997)] demonstrated that treatment with an identical mdm2 antisense oligonucleotide or a shorter version within the same region in a tumor cell line decreased DNA synthesis and cell viability and triggered apoptosis.

Chen et al. [Proc. Natl. Acad. Sci. USA, 95, 195 (1998); WO 99/10486] disclose antisense oligonucleotides targeted to the coding region of mdm2. A reduction in mdm2 RNA and protein levels was seen, and transcriptional activity from a p53-responsive promoter was increased after oligonucleotide treatment of JAR (choriocarcinoma) or SJSA (osteosarcoma) cells.

WO 93/20238 and WO 97/09343 disclose, in general, the use of antisense constructs, antisense oligonucleotides, ribozymes and triplex-forming oligonucleotides to detect or to inhibit expression of mdm2. EP 635068B1, issued Nov. 5, 1997, describes methods of treating in vitro neoplastic cells with an inhibitor of mdm2, and inhibitory compounds, including antisense oligonucleotides and triple-strand forming oligonucleotides.

There remains a long-felt need for improved compositions and methods for inhibiting mdm2 gene expression.

### SUMMARY OF THE INVENTION

The present invention provides oligonucleotide compounds, preferably antisense oligonucleotides, according to a graphical representation of a single nucleotide member thereof depicted as compound I which is further bound to any one of compounds II, III or IV. These oligonucleotides are preferably targeted to nucleic acids encoding mdm2 and are capable of modulating, and preferably, inhibiting mdm2 expression. Similarly modified oligonucleotides of the invention may also be designed which are targeted to other nucleic acid targets.

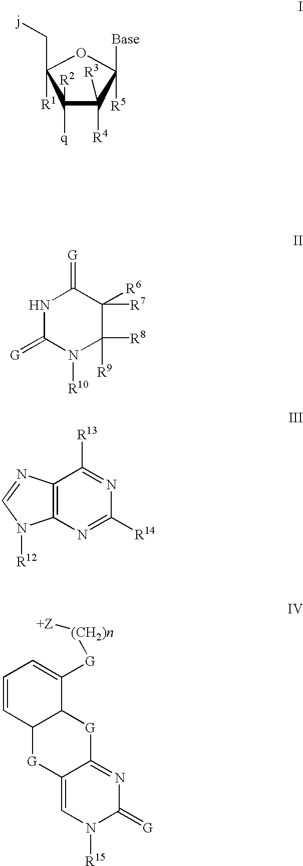

Compound I is further defined where q and j are covalent nucleoside linkers of between 1-5 atoms including carbon, nitrogen, phosphorus, sulfur and oxygen which may themselves be substituted with additional atoms not counted among the stated 1-5 atoms. The present invention also provides chimeric compounds, preferably (but not only) targeted to nucleic acids encoding mdm2. The chimeric compounds according to the present invention comprise at least one modified nucleotide according to compound I, as covalently bound to any of compounds II, III or IV.

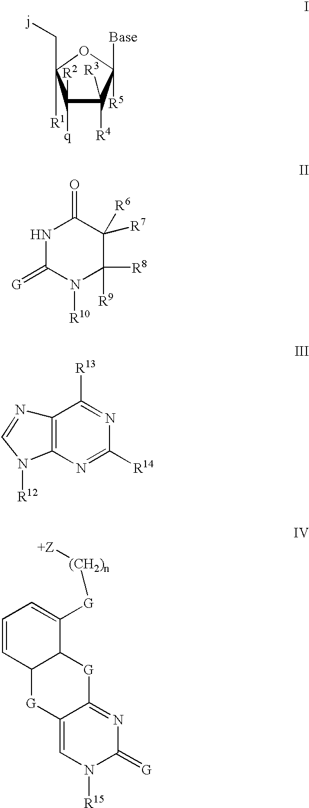

The oligonucleotide compounds of the invention are believed to be useful both diagnostically and therapeutically, and are believed to be particularly useful in the methods of the present invention.

The present invention also comprises methods of inhibiting the expression of mdm2, particularly the increased expression resulting from amplification of mdm2. These methods are believed to be useful both therapeutically and diagnostically as a consequence of the association between mdm2 expression and hyperproliferation. These methods are also useful as tools, for example, for detecting and determining the role of mdm2 expression in various cell functions and physiological processes and conditions and for diagnosing conditions associated with mdm2 expression.

The present invention also comprises methods of inhibiting hyperproliferation of cells using compounds of the invention. These methods are believed to be useful, for example, in diagnosing mdm2-associated cell hyperproliferation. Methods of treating abnormal proliferative conditions associated with mdm2 are also provided. These methods employ the antisense compounds of the invention. These methods are believed to be useful both therapeutically and as clinical research and diagnostic tools.

### DETAILED DESCRIPTION OF THE INVENTION

Tumors often result from genetic changes in cellular regulatory genes. Among the most important of these are the tumor suppressor genes, of which p53 is the most widely studied. Approximately half of all human tumors have a mutation in the p53 gene. This mutation disrupts the ability of the p53 protein to bind to DNA and act as a transcription factor. Hyperproliferation of cells occurs as a result. Another mechanism by which p53 can be inactivated is through overexpression of mdm2, which regulates p53 activity in a feedback loop. The mdm2 protein binds to p53 in its DNA binding region, preventing its activity. Mdm2 is amplified in some human tumors, and this amplification is diagnostic of neoplasia or the potential therefor. Over one third of human sarcomas have elevated mdm2 sequences. Elevated expression may also be involved in other tumors including but not limited to those in which p53 inactivation has been implicated. These include colorectal carcinoma, lung cancer and chronic myelogenous leukemia.

Many abnormal proliferative conditions, particularly hyperproliferative conditions, are believed to be associated with increased mdm2 expression and are, therefore believed to be responsive to inhibition of mdm2 expression. Examples of these hyperproliferative conditions are cancers, psoriasis, blood vessel stenosis (e.g., restenosis or atherosclerosis), and fibrosis, e.g., of the lung or kidney. Increased levels of wild-type or mutated p53 have been found in some cancers (Nagashima, G., et al., Acta Neurochir. (Wein), 1999, 141, 53-61; Fiedler, A., et al., Langenbecks Arch. Surg., 1998, 383, 269-275). Increased levels of p53 is also associated with resistance of a cancer to a chemotherapeutic drug (Brown, R., et al., Int. J. Cancer, 1993, 55, 678-684). These diseases or conditions may be amenable to treatment by induction of mdm2 expression.

The present invention employs antisense compounds, particularly oligonucleotides, for use in modulating the function of nucleic acid molecules encoding mdm2, ultimately modulating the amount of mdm2 produced. This is accomplished by providing oligonucleotides which specifically hybridize with nucleic acids, preferably mRNA, encoding mdm2.

This relationship between an antisense compound such as an oligonucleotide and its complementary nucleic acid target, to which it hybridizes, is commonly referred to as “antisense”. “Targeting” an oligonucleotide to a chosen nucleic acid target, in the context of this invention, is a multistep process. The process usually begins with identifying a nucleic acid sequence whose function is to be modulated. This may be, as examples, a cellular gene (or mRNA made from the gene) whose expression is associated with a particular disease state, or a foreign nucleic acid from an infectious agent. In the present invention, the target is a nucleic acid encoding mdm2; in other words, a mdm2 gene or RNA expressed from a mdm2 gene. mdm2 mRNA is presently the preferred target. The targeting process also includes determination of a site or sites within the nucleic acid sequence for the antisense interaction to occur such that modulation of gene expression will result.

In accordance with this invention, persons of ordinary skill in the art will understand that messenger RNA includes not only the information to encode a protein using the three letter genetic code, but also associated ribonucleotides which form a region known to such persons as the 5′-untranslated region, the 3′-untranslated region, the 5′ cap region and intron/exon junction ribonucleotides. Thus, oligonucleotides may be formulated in accordance with this invention which are targeted wholly or in part to these associated ribonucleotides as well as to the informational ribonucleotides. The oligonucleotide may therefore be specifically hybridizable with a transcription initiation site region, a translation initiation codon region, a 5′ cap region, an intron/exon junction, coding sequences, a translation termination codon region or sequences in the 5′- or 3′-untranslated region. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding mdm2, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. This region is a preferred target region. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. This region is a preferred target region. The open reading frame (ORF) or “coding region,” which is known in the art to refer to the region between the translation initiation codon and the translation termination codon, is also a region which may be targeted effectively. Other preferred target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene) and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene). mdm2 is believed to have alternative transcripts which differ in their 5′-UTR regions. The S-mdm2 transcript class is translated approximately 8-fold more efficiently than the L-mdm2 transcripts produced by the constitutive promoter. Landers et al., Cancer Res., 57, 3562 (1997). Accordingly, both the 5′-UTR of the S-mdm transcript and the 5′-UTR of the L-mdm2 transcript are preferred target regions, with the S-mdm2 5′-UTR being more preferred. mRNA splice sites may also be preferred target regions, and are particularly useful in situations where aberrant splicing is implicated in disease, or where an overproduction of a particular mRNA splice product is implicated in disease. Aberrant fusion junctions due to rearrangements or deletions may also be preferred targets.

Once the target site or sites have been identified, oligonucleotides are chosen which are sufficiently complementary to the target, i.e., hybridize sufficiently well and with sufficient specificity, to give the desired modulation.

“Hybridization”, in the context of this invention, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them.

“Specifically hybridizable” and “complementary” are terms which are used to indicate a sufficient degree of complementarity such that stable and specific binding occurs between the DNA or RNA target and the oligonucleotide.

It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment and, in the case of in vitro assays, under conditions in which the assays are conducted.

Hybridization of antisense oligonucleotides with mRNA interferes with one or more of the normal functions of mRNA. The functions of mRNA to be interfered with include all vital functions such as, for example, translocation of the RNA to the site of protein translation, translation of protein from the RNA, splicing of the RNA to yield one or more mRNA species, and catalytic activity which may be engaged in by the RNA.

The overall effect of interference with mRNA function is modulation of mdm2 expression. In the context of this invention “modulation” means either inhibition or stimulation; i.e., either a decrease or increase in expression. This modulation can be measured in ways which are routine in the art, for example by Northern blot assay of mRNA expression as taught in the examples of the instant application or by Western blot or ELISA assay of protein expression, or by an immunoprecipitation assay of protein expression, as taught in the examples of the instant application. Effects on cell proliferation or tumor cell growth can also be measured, as taught in the examples of the instant application.

The antisense compounds of this invention can be used in diagnostics, therapeutics, prophylaxis, and as research reagents and in kits. Since these compounds hybridize to nucleic acids encoding mdm2, sandwich, calorimetric and other assays can easily be constructed to exploit this fact. Furthermore, since the antisense compounds of this invention hybridize specifically to nucleic acids encoding particular isozymes of mdm2, such assays can be devised for screening of cells and tissues for particular mdm2 isozymes. Such assays can be utilized for diagnosis of diseases associated with various mdm2 forms. Provision of means for detecting hybridization of oligonucleotide with a mdm2 gene or mRNA can routinely be accomplished. Such provision may include enzyme conjugation, radiolabelling or any other suitable detection systems. Kits for detecting the presence or absence of mdm2 may also be prepared.

The present invention is also suitable for diagnosing abnormal proliferative states in tissue or other samples from patients suspected of having a hyperproliferative disease such as cancer or psoriasis. The ability of the oligonucleotides of the present invention to inhibit cell proliferation may be employed to diagnose such states. A number of assays may be formulated employing the present invention, which assays will commonly comprise contacting a tissue sample with an antisense compound of the invention under conditions selected to permit detection and, usually, quantitation of such inhibition. In the context of this invention, to “contact” tissues or cells with an antisense compound means to add the compound(s), usually in a liquid carrier, to a cell suspension or tissue sample, either in vitro or ex vivo, or to administer the antisense compound(s) to cells or tissues within an animal. Similarly, the present invention can be used to distinguish mdm2-associated tumors, particularly tumors associated with mdm2α, from tumors having other etiologies, in order that an efficacious treatment regime can be designed.

The antisense compounds of this invention may also be used for research purposes. Thus, the specific hybridization exhibited by oligonucleotides may be used for assays, purifications, cellular product preparations and in other methodologies which may be appreciated by persons of ordinary skill in the art.

In the context of this invention, the term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

The antisense compounds in accordance with this invention preferably comprise from about 5 to about 50 nucleobases. Particularly preferred are antisense oligonucleotides comprising from about 8 to about 30 linked nucleobases (i.e. from about 8 to about 30 nucleosides). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thiono-alkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. No.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

Representative United States patents that teach the preparation of the above oligonucleosides include, but are not limited to, U.S. Pat. No.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Specific examples of some preferred modified oligonucleotides envisioned for this invention include those containing phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioates (usually abbreviated in the art as P═S) and those with CH₂—NH—O—CH₂, CH₂—N(CH₃)—O—CH₂ [known as a methylene(methylimino) or MMI backbone], CH₂—O—N(CH₃) —CH₂, CH₂—N(CH₃) —N(CH₃) —CH₂ and O—N(CH₃) —CH₂—CH₂ backbones, wherein the native phosphodiester (usually abbreviated in the art as P═O) backbone is represented as O—P—O—CH₂). Also preferred are oligonucleotides having morpholino backbone structures (Summerton and Weller, U.S. Pat. No. 5,034,506). Further preferred are oligonucleotides with NR—C(*)—CH₂—CH₂, CH₂—NR—C(*) —CH₂, CH₂—CH₂—NR—C(*), C(*)—NR—CH₂—CH₂ and CH₂—C(*)—NR—CH₂ backbones, wherein “*” represents O or S (known as amide backbones; DeMesmaeker et al., WO 92/20823, published Nov. 26, 1992).

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. No.: 5,539,082; 5,714,331; and 5,719,262, each of which is herein incorporated by reference. Further teaching of PNA compounds can be found in Nielsen et al., Science, 1991, 254, 1497-1500.

A further preferred modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH2—)n group bridging the 2′ oxygen atom and the 4′ carbon atom wherein n is 1 or 2. LNAs and preparation thereof are described in WO 98/39352 and WO 99/14226.

Preferred modified oligonucleotides may contain one or more substituted sugar moieties comprising one of the following at the 2′ position: OH, SH, SCH₃, F, OCN, OCH30CH₃, OCH₃O (CH₂)ₙCH₃, O(CH₂)ₙNH₂ or O(CH₂)ₙCH3 where n is from 1 to about 10; C₁ to C10 lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-O-methoxyethyl [which can be written as 2′-O-CH₂CH₂OCH3, and is also known in the art as 2′-O-(2-methoxyethyl) or 2′-methoxyethoxy] [Martin et al., Helv. Chim. Acta, 78, 486 (1995)]. Other preferred modifications include 2′-methoxy (2′-O-CH₃), 2′-propoxy (2′-OCH₂CH₂CH₃), 2′-aminopropoxy (2′-OCH₂CH₂CH₂NH₂) and 2′-fluoro (2′-F). A further preferred modification includes 2′-dimethylaminooxyethoxy, i.e., a O(CH₂)₂ON(CH₃)₂ group, also known as 2′-DMAOE, as described in examples hereinbelow. Similar modifications may also be made at other positions on the oligonucleotide, particularly the 3′ position of the sugar on the 3′ terminal nucleotide or in 2′-5′ linked oligonucleotides, and the 5′ position of the 5′ terminal nucleotide. Oligonucleotides may also have sugar mimetics such as cyclobutyls in place of the pentofuranosyl group.

Other preferred modifications include 2′-methoxy (2′—O—CH₃), 2′-aminopropoxy (2′—OCH₂CH₂CH₂NH₂), 2′-allyl (2′-CH₂—CH═CH₂), 2′-O-allyl (2′—O—CH₂—CH═CH₂) and 2′-fluoro (2′—F). The 2′-modification may be in the arabino (up) position or ribo (down) position. A preferred 2′-arabino modification is 2′—F. Representative United States patents that teach the preparation of modified sugar structures include, but are not limited to, U.S. Pat. No.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

The oligonucleotides of the invention may additionally or alternatively include nucleobase modifications or substitutions. As used herein, “unmodified” or “natural” nucleobases include adenine (A), guanine (G), thymine (T), cytosine (C) and uracil (U). Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 5-hydroxymethyluracil, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. N⁶(6-aminohexyl)adenine and 2,6-diaminopurine are also included. [Kornberg, A., DNA Replication, 1974, W. H. Freeman & Co., San Francisco, 1974, pp. 75-77; Gebeyehu, G., et al., Nucleic Acids Res., 15, 4513 (1987)]. Further modified nucleobases include tricyclic pyrimidines such as phenoxazine cytidine(1H-pyrimido[5,4-b] [1,4]benzoxazin-2(3H)-one), phenothiazine cytidine (1H-pyrimido[5,4-b] [1,4]benzothiazin-2(3H)-one), G-clamps such as a substituted phenoxazine cytidine (e.g. 9-(2-aminoethoxy)-H-pyrimido[5,4-b] [1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyl-adenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. No.: 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Another preferred additional or alternative modification of the oligonucleotides of the invention involves chemically linking to the oligonucleotide one or more lipophilic moieties which enhance the cellular uptake of the oligonucleotide. Such lipophilic moieties may be linked to an oligonucleotide at several different positions on the oligonucleotide. Some preferred positions include the 3′ position of the sugar of the 3′ terminal nucleotide, the 5′ position of the sugar of the 5′ terminal nucleotide, and the 2′ position of the sugar of any nucleotide. The N6 position of a purine nucleobase may also be utilized to link a lipophilic moiety to an oligonucleotide of the invention (Gebeyehu, G., et al., Nucleic Acids Res., 1987, 15, 4513). Such lipophilic moieties include but are not limited to a cholesteryl moiety [Letsinger et al., Proc. Natl. Acad. Sci. USA,, 86, 6553 (1989)], cholic acid [Manoharan et al., Bioorg. Med. Chem. Let., 4, 1053 (1994)], a thioether, e.g., hexyl-S-tritylthiol [Manoharan et al., Ann. N.Y. Acad. Sci., 660, 306 (1992); Manoharan et al., Bioorg. Med. Chem. Let., 3, 2765 (1993)], a thiocholesterol [Oberhauser et al., Nucl. Acids Res., 20, 533 (1992)], an aliphatic chain, e.g., dodecandiol or undecyl residues [Saison-Behmoaras et al., EMBO J., 10, 111 (1991); Kabanov et al., FEBS Lett., 259, 327 (1990); Svinarchuk et al., Biochimie., 75, 49(1993)], a phospholipid, e.g., di-hexadecyl-rac-glycerol or triethylammonium 1,2-di-O-hexadecyl-rac-glycero-3-H-phosphonate [Manoharan et al., Tetrahedron Lett., 36, 3651 (1995); Shea et al., Nucl. Acids Res., 18, 3777 (1990)], a polyamine or a polyethylene glycol chain [Manoharan et al., Nucleosides & Nucleotides, 14, 969 (1995)], or adamantane acetic acid [Manoharan et al., Tetrahedron Lett., 36, 3651 (1995)], a palmityl moiety [Mishra et al., Biochim. Biophys. Acta, 1264, 229 (1995)], or an octadecylamine or hexylamino-carbonyl-oxycholesterol moiety [Crooke et al., J. Pharmacol. Exp. Ther., 277, 923 (1996)]. Oligonucleotides comprising lipophilic moieties, and methods for preparing such oligonucleotides, as disclosed in U.S. Pat. No. 5,138,045, No. 5,218,105 and No. 5,459,255, the contents of which are hereby incorporated by reference.

The present invention also includes oligonucleotides which are chimeric oligonucleotides. “Chimeric” oligonucleotides or “chimeras,” in the context of this invention, are oligonucleotides which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Cleavage of the RNA target can be routinely detected by gel electrophoresis and, if necessary, associated nucleic acid hybridization techniques known in the art. This RNAse H-mediated cleavage of the RNA target is distinct from the use of ribozymes to cleave nucleic acids.

Examples of chimeric oligonucleotides include but are not limited to “gapmers,” in which three distinct regions are present, normally with a central region flanked by two regions which are chemically equivalent to each other but distinct from the gap. A preferred example of a gapmer is an oligonucleotide in which a central portion (the “gap”) of the oligonucleotide serves as a substrate for RNase H and is preferably composed of 2′-deoxynucleotides, while the flanking portions (the 5′ and 3′ “wings”) are modified to have greater affinity for the target RNA molecule but are unable to support nuclease activity (e.g., 2′-fluoro-or 2′-O-methoxyethyl- substituted). Other chimeras include “wingmers,” also known in the art as “hemimers,” that is, oligonucleotides with two distinct regions. In a preferred example of a wingmer, the 5′ portion of the oligonucleotide serves as a substrate for RNase H and is preferably composed of 2′-deoxynucleotides, whereas the 3′ portion is modified in such a fashion so as to have greater affinity for the target RNA molecule but is unable to support nuclease activity (e.g., 2′-fluoro- or 2′-O-methoxyethyl- substituted), or vice-versa. In one embodiment, the oligonucleotides of the present invention contain a 2′-O-methoxyethyl (2′-O-CH2CH20CH3) modification on the sugar moiety of at least one nucleotide. This modification has been shown to increase both affinity of the oligonucleotide for its target and nuclease resistance of the oligonucleotide. According to the invention, one, a plurality, or all of the nucleotide subunits of the oligonucleotides of the invention may bear a 2′-O-methoxyethyl (—O—CH2CH2OCH3) modification. oligonucleotides comprising a plurality of nucleotide subunits having a 2′-O-methoxyethyl modification can have such a modification on any of the nucleotide subunits within the oligonucleotide, and may be chimeric oligonucleotides. Aside from or in addition to 2′-O-methoxyethyl modifications, oligonucleotides containing other modifications which enhance antisense efficacy, potency or target affinity are also preferred. Chimeric oligonucleotides comprising one or more such modifications are presently preferred. Through use of such modifications, active oligonucleotides have been identified which are shorter than conventional “first generation” oligonucleotides active against mdm2. oligonucleotides in accordance with this invention are from 5 to 50 nucleotides in length, preferably from about 8 to about 30. In the context of this invention it is understood that this encompasses non-naturally occurring oligomers as hereinbefore described, having from 5 to 50 monomers, preferably from about 8 to about 30.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of the routineer. It is well known to use similar techniques to prepare oligonucleotides such as the phosphorothioates and 2′-alkoxy or 2′-alkoxyalkoxy derivatives, including 2′-O-methoxyethyl oligonucleotides [Martin, P., Helv. Chim. Acta, 78, 486 (1995)]. It is also well known to use similar techniques and commercially available modified amidites and controlled-pore glass (CPG) products such as biotin, fluorescein, acridine or psoralen-modified amidites and/or CPG (available from Glen Research, Sterling Va.) to synthesize fluorescently labeled, biotinylated or other conjugated oligonucleotides.

The antisense compounds of the present invention include bioequivalent compounds, including pharmaceutically acceptable salts and prodrugs. This is intended to encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to pharmaceutically acceptable salts of the nucleic acids of the invention and prodrugs of such nucleic acids.

Pharmaceutically acceptable “salts” are physiologically and pharmaceutically acceptable salts of the nucleic acids of the invention: i.e., salts that retain the desired biological activity of the parent compound and do not impart undesired toxicological effects thereto [see, for example, Berge et al., “Pharmaceutical Salts,” J. of Pharma Sci., 66:1 (1977)].

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; 8 salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The oligonucleotides of the invention may additionally or alternatively be prepared to be delivered in a “prodrug” form. The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993.

For therapeutic or prophylactic treatment, oligonucleotides are administered in accordance with this invention. oligonucleotide compounds of the invention may be formulated in a pharmaceutical composition, which may include pharmaceutically acceptable carriers, thickeners, diluents, buffers, preservatives, surface active agents, neutral or cationic lipids, lipid complexes, liposomes, penetration enhancers, carrier compounds and other pharmaceutically acceptable carriers or excipients and the like in addition to the oligonucleotide. Such compositions and formulations are comprehended by the present invention.

Pharmaceutical compositions comprising the oligonucleotides of the present invention may include penetration enhancers in order to enhance the alimentary delivery of the oligonucleotides. Penetration enhancers may be classified as belonging to one of five broad categories, i.e., fatty acids, bile salts, chelating agents, surfactants and non-surfactants (Lee et al., Critical Reviews in Therapeutic Drug Carrier Systems, 1991, 8:91-192; Muranishi, Critical Reviews in Therapeutic Drug Carrier Systems, 1990, 7:1). One or more penetration enhancers from one or more of these broad categories may be included. Compositions comprising oligonucleotides and penetration enhancers are disclosed in co-pending U.S. patent application Ser. No. 08/886,829 to Teng et al., filed Jul. 1, 1997, which is herein incorporated by reference in its entirety.

The compositions of the present invention may additionally contain other adjunct components conventionally found in pharmaceutical compositions, at their art-established usage levels. Thus, for example, the compositions may contain additional compatible pharmaceutically-active materials such as, e.g., antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the invention.

Regardless of the method by which the oligonucleotides of the invention are introduced into a patient, colloidal dispersion systems may be used as delivery vehicles to enhance the in vivo stability of the oligonucleotides and/or to target the oligonucleotides to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid: oligonucleotide complexes of uncharacterized structure. A preferred colloidal dispersion system is a plurality of liposomes. Liposomes are microscopic spheres having an aqueous core surrounded by one or more outer layers made up of lipids arranged in a bilayer configuration [see, generally, Chonn et al., Current Op. Biotech., 6, 698 (1995)]. Liposomal antisense compositions are prepared according to the disclosure of co-pending U.S. patent application Ser. No. 08/961,469 to Hardee et al., filed Oct. 31, 1997, herein incorporated by reference in its entirety.

The pharmaceutical compositions of the present invention may be administered in a number of ways depending upon whether local or systemic treatment is desired and upon the area to be treated. Administration may be topical (including ophthalmic, vaginal, rectal, intranasal, epidermal and transdermal), oral or parenteral. Parenteral administration includes intravenous drip, subcutaneous, intraperitoneal or intramuscular injection, pulmonary administration, e.g., by inhalation or insufflation, or intracranial, e.g., intrathecal or intraventricular, administration. Oligonucleotides with at least one 2′-O-methoxyethyl modification are believed to be particularly useful for oral administration. Modes of administering oligonucleotides are disclosed in co-pending U.S. patent application Ser. No. 08/961,469 to Hardee et al., filed Oct. 31, 1997, herein incorporated by reference in its entirety.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water or non-aqueous media, capsules, sachets or tablets. Thickeners, flavoring agents, diluents, emulsifiers, dispersing aids or binders may be desirable.

Compositions for parenteral administration may include sterile aqueous solutions which may also contain buffers, diluents and other suitable additives. In some cases it may be more effective to treat a patient with an oligonucleotide of the invention in conjunction with other traditional therapeutic modalities in order to increase the efficacy of a treatment regimen. In the context of the invention, the term “treatment regimen” is meant to encompass therapeutic, palliative and prophylactic modalities. For example, a patient may be treated with conventional chemotherapeutic agents, particularly those used for tumor and cancer treatment. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine (CA), 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine,taxol,vincristine,vinblastine,etoposide, trimetrexate, teniposide, cisplatin and diethylstilbestrol (DES). See, generally, The Merck Manual of Diagnosis and Therapy, 15th Ed., pp. 1206-1228, Berkow et al., eds., Rahay, N.J., 1987). When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide).

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC50s found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

Thus, in the context of this invention, by “therapeutically effective amount” is meant the amount of the compound which is required to have a therapeutic effect on the treated mammal. This amount, which will be apparent to the skilled artisan, will depend upon the type of mammal, the age and weight of the mammal, the type of disease to be treated, perhaps even the gender of the mammal, and other factors which are routinely taken into consideration when treating a mammal with a disease. A therapeutic effect is assessed in the mammal by measuring the effect of the compound on the disease state in the animal. For example, if the disease to be treated is cancer, therapeutic effects are assessed by measuring the rate of growth or the size of the tumor, or by measuring the production of compounds such as cytokines, production of which is an indication of the progress or regression of the tumor.

The following examples illustrate the present invention and are not intended to limit the same.

### EXAMPLES

### Example 1

Synthesis of Oligonucleotides

Unmodified oligodeoxynucleotides are synthesized on an automated DNA synthesizer (Applied Biosystems model 380B) using standard phosphoramidite chemistry with oxidation by iodine. -cyanoethyldiisopropyl-phosphoramidites are purchased from Applied Biosystems (Foster City, Calif.). For phosphorothioate oligonucleotides, the standard oxidation bottle was replaced by a 0.2 M solution of 3H-1,2-benzodithiole-3-one 1,1-dioxide in acetonitrile for the stepwise thiation of the phosphite linkages. The thiation cycle wait step was increased to 68 seconds and was followed by the capping step.

2′-methoxy oligonucleotides are synthesized using 2′-methoxy -cyanoethyldiisopropyl-phosphoramidites (Chemgenes, Needham, Mass.) and the standard cycle for unmodified oligonucleotides, except the wait step after pulse delivery of tetrazole and base was increased to 360 seconds. Other 2′-alkoxy oligonucleotides were synthesized by a modification of this method, using appropriate 2′-modified amidites such as those available from Glen Research, Inc., Sterling, Va.

2′-fluoro oligonucleotides were synthesized as described in Kawasaki et al., J. Med. Chem., 36, 831 (1993). Briefly, the protected nucleoside N6-benzoyl-2′-deoxy-2′-fluoroadenosine was synthesized utilizing commercially available 9-β-D-arabinofuranosyladenine as starting material and by modifying literature procedures whereby the 2′- -fluoro atom is introduced by a SN2-displacement of a 2′-β-O-trifyl group. Thus N6-benzoyl-9-β-D-arabinofuranosyladenine was selectively protected in moderate yield as the 3′,5′-ditetrahydropyranyl (THP) intermediate. Deprotection of the THP and N6-benzoyl groups was accomplished using standard methodologies and standard methods were used to obtain the 5′-dimethoxytrityl- (DMT) and 5′-DMT-3′-phosphoramidite intermediates.

The synthesis of 2′-deoxy-2′-fluoroguanosine was accomplished using tetraisopropyldisiloxanyl (TPDS) protected 9-β-D-arabinofuranosylguanine as starting material, and conversion to the intermediate diisobutyryl-arabinofuranosylguanosine. Deprotection of the TPDS group was followed by protection of the hydroxyl group with THP to give diisobutyryl di-THP protected arabinofuranosylguanine. Selective O-deacylation and triflation was followed by treatment of the crude product with fluoride, then deprotection of the THP groups. Standard methodologies were used to obtain the 5′-DMT- and 5′-DMT-3′-phosphoramidites.

Synthesis of 2′-deoxy-2′-fluorouridine was accomplished by the modification of a known procedure in which 2, 2′-anhydro-1-β-D-arabinofuranosyluracil was treated with 70% hydrogen fluoride-pyridine. Standard procedures were used to obtain the 5′-DMT and 5′-DMT-3′phosphoramidites.

2′-deoxy-2′-fluorocytidine was synthesized via amination of 2′-deoxy-2′-fluorouridine, followed by selective protection to give N4-benzoyl-2′-deoxy-2′-fluorocytidine. Standard procedures were used to obtain the 5′-DMT and 5′-DMT-3′phosphoramidites.

2′-(2-methoxyethyl)-modified amidites are synthesized according to Martin, P., Helv. Chim. Acta, 78,486 (1995). For ease of synthesis, the last nucleotide was a deoxynucleotide. 2′-O-CH₂CH₂OCH₃-cytosines may be 5-methyl cytosines.

Synthesis of 5-Methyl cytosine monomers:

2,2′-Anhydro[1-(-D-arabinofuranosyl)-5-methyluridine]:

5-Methyluridine (ribosylthymine, commercially available through Yamasa, Choshi, Japan) (72.0 g, 0.279 M), diphenylcarbonate (90.0 g, 0.420 M) and sodium bicarbonate (2.0 g, 0.024 M) were added to DMF (300 mL). The mixture was heated to reflux, with stirring, allowing the evolved carbon dioxide gas to be released in a controlled manner. After 1 hour, the slightly darkened solution was concentrated under reduced pressure. The resulting syrup was poured into diethylether (2.5 L), with stirring. The product formed a gum. The ether was decanted and the residue was dissolved in a minimum amount of methanol (ca. 400 mL). The solution was poured into fresh ether (2.5 L) to yield a stiff gum. The ether was decanted and the gum was dried in a vacuum oven (60° C. at 1 mm Hg for 24 hours) to give a solid which was crushed to a light tan powder (57 g, 85% crude yield). The material was used as is for further reactions.

2′-O-Methoxyethyl-5-methyluridine:

2,2′-Anhydro-5-methyluridine (195 g, 0.81 M), tris(2-methoxyethyl)borate (231 g, 0.98 M) and 2-methoxyethanol (1.2 L) were added to a 2 L stainless steel pressure vessel and placed in a pre-heated oil bath at 160° C. After heating for 48 hours at 155-160° C., the vessel was opened and the solution evaporated to dryness and triturated with MeOH (200 mL). The residue was suspended in hot acetone (1 L). The insoluble salts were filtered, washed with acetone (150 mL) and the filtrate evaporated. The residue (280 g) was dissolved in CH₃CN (600 mL) and evaporated. A silica gel column (3 kg) was packed in CH₂Cl₂/acetone/MeOH (20:5:3) containing 0.5% Et₃NH. The residue was dissolved in CH₂Cl₂ (250 mL) and adsorbed onto silica (150 g) prior to loading onto the column. The product was eluted with the packing solvent to give 160 g (63%) of product.

2′-O-Methoxyethyl-5′-O-dimethoxytrityl-5-methyluridine:

2′-O-Methoxyethyl-5-methyluridine (160 g, 0.506 M) was co-evaporated with pyridine (250 mL) and the dried residue dissolved in pyridine (1.3 L). A first aliquot of dimethoxytrityl chloride (94.3 g, 0.278 M) was added and the mixture stirred at room temperature for one hour. A second aliquot of dimethoxytrityl chloride (94.3 g, 0.278 M) was added and the reaction stirred for an additional one hour. Methanol (170 mL) was then added to stop the reaction. HPLC showed the presence of approximately 70% product. The solvent was evaporated and triturated with CH3CN (200 mL). The residue was dissolved in CHCl3 (1.5 L) and extracted with 2×500 mL of saturated NaHCO3 and 2×500 mL of saturated NaCl. The organic phase was dried over Na2SO4, filtered and evaporated. 275 g of residue was obtained. The residue was purified on a 3.5 kg silica gel column, packed and eluted with EtOAc/Hexane/Acetone (5:5:1) containing 0.5% Et3NH. The pure fractions were evaporated to give 164 g of product. Approximately 20 g additional was obtained from the impure fractions to give a total yield of 183 g (57%).

3′-O-Acetyl-2′-O-methoxyethyl-5′-O-dimethoxytrityl-5-methyluridine:

2′-O-Methoxyethyl-5′-O-dimethoxytrityl-5-methyl-uridine (106 g, 0.167 M), DMF/pyridine (750 mL of a 3:1 mixture prepared from 562 mL of DMF and 188 mL of pyridine) and acetic anhydride (24.38 mL, 0.258 M) were combined and stirred at room temperature for 24 hours. The reaction was monitored by tic by first quenching the tic sample with the addition of MeOH. Upon completion of the reaction, as judged by tic, MeOH (50 mL) was added and the mixture evaporated at 35° C. The residue was dissolved in CHCl3 (800 mL) and extracted with 2×200 mL of saturated sodium bicarbonate and 2×200 mL of saturated NaCl. The water layers were back extracted with 200 mL of CHCl3. The combined organics were dried with sodium sulfate and evaporated to give 122 g of residue (approx. 90% product). The residue was purified on a 3.5 kg silica gel column and eluted using EtOAc/Hexane(4:1). Pure product fractions were evaporated to yield 96 g (84%).

3′-O-Acetyl-2′-O-methoxyethyl-5′-O-dimethoxytrityl-5-methyl-4-triazoleuridine:

A first solution was prepared by dissolving 3′-O-acetyl-2′-O-methoxyethyl-5′-O-dimethoxytrityl-5-methyluridine (96 g, 0.144 M) in CH3CN (700 mL) and set aside. Triethylamine (189 mL, 1.44 M) was added to a solution of triazole (90 g, 1.3 M) in CH3CN (1 L), cooled to −5° C. and stirred for 0.5 h using an overhead stirrer. POCl3 was added dropwise, over a 30 minute period, to the stirred solution maintained at 0-10° C., and the resulting mixture stirred for an additional 2 hours. The first solution was added dropwise, over a 45 minute period, to the later solution. The resulting reaction mixture was stored overnight in a cold room. Salts were filtered from the reaction mixture and the solution was evaporated. The residue was dissolved in EtOAc (1 L) and the insoluble solids were removed by filtration. The filtrate was washed with 1×300 mL of NaHCO3 and 2×300 mL of saturated NaCl, dried over sodium sulfate and evaporated. The residue was triturated with EtOAc to give the title compound.

2′-O-Methoxyethyl-5′-O-dimethoxytrityl-5-methylcytidine:

A solution of 3′-O-acetyl-2′-O-methoxyethyl-5′-O-dimethoxytrityl-5-methyl-4-triazoleuridine (103 g, 0.141 M) in dioxane (500 mL) and NH40H (30 mL) was stirred at room temperature for 2 hours. The dioxane solution was evaporated and the residue azeotroped with MeOH (2×200 mL). The residue was dissolved in MeOH (300 mL) and transferred to a 2 liter stainless steel pressure vessel. MeOH (400 mL) saturated with NH3 gas was added and the vessel heated to 100° C. for 2 hours (tlc showed complete conversion). The vessel contents were evaporated to dryness and the residue was dissolved in EtOAc (500 mL) and washed once with saturated NaCl (200 mL). The organics were dried over sodium sulfate and the solvent was evaporated to give 85 g (95%) of the title compound.

N4-Benzoyl-2′-O-methoxyethyl-5′-O-dimethoxytrityl-5-methylcytidine:

2′-O-Methoxyethyl-5′-O-dimethoxytrityl-5-methyl-cytidine (85 g, 0.134 M) was dissolved in DMF (800 mL) and benzoic anhydride (37.2 g, 0.165 M) was added with stirring. After stirring for 3 hours, tlc showed the reaction to be approximately 95% complete. The solvent was evaporated and the residue azeotroped with MeOH (200 mL). The residue was dissolved in CHCl3 (700 mL) and extracted with saturated NaHCO3 (2×300 mL) and saturated NaCl (2×300 mL), dried over MgSO4 and evaporated to give a residue (96 g). The residue was chromatographed on a 1.5 kg silica column using EtOAc/Hexane (1:1) containing 0.5% Et3NH as the eluting solvent. The pure product fractions were evaporated to give 90 g (90%) of the title compound.

N4-Benzoyl-2′-O-methoxyethyl-5′-O-dimethoxytrityl-5-methylcytidine-3′-amidite:

N4-Benzoyl-2′-O-methoxyethyl-5′-O-dimethoxytrityl-5-methylcytidine (74 g, 0.10 M) was dissolved in CH2Cl2 (1 L). Tetrazole diisopropylamine (7.1 g) and 2-cyanoethoxy-tetra(isopropyl)phosphite (40.5 mL, 0.123 M) were added with stirring, under a nitrogen atmosphere. The resulting mixture was stirred for 20 hours at room temperature (tlc showed the reaction to be 95% complete). The reaction mixture was extracted with saturated NaHCO3 (1×300 mL) and saturated NaCl (3×300 mL). The aqueous washes were back-extracted with CH2Cl2 (300 mL), and the extracts were combined, dried over MgSO4 and concentrated. The residue obtained was chromatographed on a 1.5 kg silica column using EtOAc\Hexane (3:1) as the eluting solvent. The pure fractions were combined to give 90.6 g (87%) of the title compound.

5-methyl-2′-deoxycytidine (5-me-C) containing oligonucleotides were synthesized according to published methods [Sanghvi et al., Nucl. Acids Res., 21, 3197 (1993)] using commercially available phosphoramidites (Glen Research, Sterling Va. or ChemGenes, Needham Mass.).

2═—O-(dimethylaminooxyethyl) nucleoside amidites

2′-(Dimethylaminooxyethoxy) nucleoside amidites [also known in the art as 2′-O-(dimethylaminooxyethyl) nucleoside amidites] are prepared as described in the following paragraphs. Adenosine, cytidine and guanosine nucleoside amidites are prepared similarly to the thymidine (5-methyluridine) except the exocyclic amines are protected with a benzoyl moiety in the case of adenosine and cytidine and with isobutyryl in the case of guanosine.

5′-O-tert-butyldiphenylsilyl-O2-2′-anhydro-5-methyluridine

O2-2′-anhydro-5-methyluridine (Pro. Bio. Sint., Varese, Italy, 100.0 g, 0.416 mmol), dimethylaminopyridine (0.66 g, 0.013 eq, 0.0054 mmol) were dissolved in dry pyridine (500 mL) at ambient temperature under an argon atmosphere and with mechanical stirring. tert-butyldiphenylchlorosilane (125.8 g, 119.0 mL, 1.1 eq, 0.458 mmol) was added in one portion. The reaction was stirred for 16 h at ambient temperature. TLC (Rf 0.22, ethyl acetate) indicated a complete reaction. The solution was concentrated under reduced pressure to a thick oil. This was partitioned between dichloromethane (1 L) and saturated sodium bicarbonate (2×1 L) and brine (1 L). The organic layer was dried over sodium sulfate and concentrated under reduced pressure to a thick oil. The oil was dissolved in a 1:1 mixture of ethyl acetate and ethyl ether (600 mL) and the solution was cooled to −10° C. The resulting crystalline product was collected by filtration, washed with ethyl ether (3×200 mL) and dried (40° C., 1 mm Hg, 24 h) to 149 g (74.8%) of white solid. TLC and NMR were consistent with pure product.

5′-O-tert-butyldiphenylsilyl-2′-O-(2-hydroxyethyl)-5-methyluridine

In a 2 L stainless steel, unstirred pressure reactor was added borane in tetrahydrofuran (1.0 M, 2.0 eq, 622 mL). In the fume hood and with manual stirring, ethylene glycol (350 mL, excess) was added cautiously at first until the evolution of hydrogen gas subsided. 5′-O-tert-butyldiphenylsilyl-O2-2′-anhydro-5-methyluridine (149 g, 0.311 mol) and sodium bicarbonate (0.074 g, 0.003 eq) were added with manual stirring. The reactor was sealed and heated in an oil bath until an internal temperature of 160° C. was reached and then maintained for 16 h (pressure<100 psig). The reaction vessel was cooled to ambient and opened. TLC (Rf 0.67 for desired product and Rf 0.82 for ara-T side product, ethyl acetate) indicated about 70% conversion to the product. In order to avoid additional side product formation, the reaction was stopped, concentrated under reduced pressure (10 to 1 mm Hg) in a warm water bath (40-100° C.) with the more extreme conditions used to remove the ethylene glycol. [Alternatively, once the low boiling solvent is gone, the remaining solution can be partitioned between ethyl acetate and water. The product will be in the organic phase.] The residue was purified by column chromatography (2 kg silica gel, ethyl acetate-hexanes gradient 1:1 to 4:1). The appropriate fractions were combined, stripped and dried to product as a white crisp foam (84 g, 50%), contaminated starting material (17.4 g) and pure reusable starting material 20 g. The yield based on starting material less pure recovered starting material was 58%. TLC and NMR were consistent with 99% pure product.

2′-O-([2-phthalimidoxy)ethyl]-5′-t-butyldiphenylsilyl-5-methyluridine

5′-O-tert-butyldiphenylsilyl-2′-O-(2-hydroxyethyl)-5-methyluridine (20 g, 36.98 mmol) was mixed with triphenylphosphine (11.63 g, 44.36 mmol) and N-hydroxyphthalimide (7.24 g, 44.36 mmol). It was then dried over P205 under high vacuum for two days at 40° C. The reaction mixture was flushed with argon and dry THF (369.8 mL, Aldrich, sure seal bottle) was added to get a clear solution. Diethyl-azodicarboxylate (6.98 mL, 44.36 mmol) was added dropwise to the reaction mixture. The rate of addition is maintained such that resulting deep red coloration is just discharged before adding the next drop. After the addition was complete, the reaction was stirred for 4 hrs. By that time TLC showed the completion of the reaction (ethylacetate:hexane, 60:40). The solvent was evaporated in vacuum. Residue obtained was placed on a flash column and eluted with ethyl acetate:hexane (60:40), to get 2′-O-([2-phthalimidoxy)ethyl]-5′-t-butyldiphenylsilyl-5-methyluridine as white foam (21.819, 86%).

5′-O-tert-butyldiphenylsilyl-2′-O-[(2-formadoximinooxy)ethyl]-5-methyluridine

2′-O-([2-phthalimidoxy)ethyl]-5′-t-butyldiphenylsilyl-5-methyluridine (3.19, 4.5 mmol) was dissolved in dry CH2Cl2 (4.5 mL) and methylhydrazine (300 mL, 4.64 mmol) was added dropwise at −10° C. to 0° C. After 1 hr the mixture was filtered, the filtrate was washed with ice cold CH2Cl2 and the combined organic phase was washed with water, brine and dried over anhydrous Na₂SO₄. The solution was concentrated to get 2′-O-(aminooxyethyl) thymidine, which was then dissolved in MeOH (67.5 mL). To this formaldehyde (20% aqueous solution, w/w, 1.1 eg.) was added and the mixture for 1 hr. Solvent was removed under vacuum; residue chromatographed to get 5′-O-tert-butyldiphenylsilyl-2′-O-[(2-formadoximinooxy) ethyl]-5-methyluridine as white foam (1.95, 78%).

5′-O-tert-butyldiphenylsilyl-2′-O-[N,N-dimethylaminooxyethyl]-5-methyluridine

5′-O-tert-butyldiphenylsilyl-2′-O-[(2-formadoximinooxy)ethyl]-5-methyluridine (1.77 g, 3.12 mmol) was dissolved in a solution of 1M pyridinium p-toluenesulfonate (PPTS) in dry MeOH (30.6 mL). Sodium cyanoborohydride (0.39 g, 6.13 mmol) was added to this solution at 10° C. under inert atmosphere. The reaction mixture was stirred for 10 minutes at 10° C. After that the reaction vessel was removed from the ice bath and stirred at room temperature for 2 hr, the reaction monitored by TLC (5% MeOH in CH₂Cl₂) . Aqueous NaHCO₃ solution (5%, 10 mL) was added and extracted with ethyl acetate (2×20 mL). Ethyl acetate phase was dried over anhydrous Na₂SO₄, evaporated to dryness. Residue was dissolved in a solution of 1M PPTS in MeOH (30.6 mL). Formaldehyde (20% w/w, 30 mL, 3.37 mmol) was added and the reaction mixture was stirred at room temperature for 10 minutes. Reaction mixture cooled to 10° C. in an ice bath, sodium cyanoborohydride (0.39 g, 6.13 mmol) was added and reaction mixture stirred at 10° C. for 10 minutes. After 10 minutes, the reaction mixture was removed from the ice bath and stirred at room temperature for 2 hrs. To the reaction mixture 5% NaHCO3 (25 mL) solution was added and extracted with ethyl acetate (2×25 mL). Ethyl acetate layer was dried over anhydrous Na₂SO₄ and evaporated to dryness. The residue obtained was purified by flash column chromatography and eluted with 5% MeOH in CH₂Cl₂ to get 5′-O-tert-butyldiphenylsilyl-2′-O-[N,N-dimethylaminooxyethyl]-5-methyluridine as a white foam (14.6 g, 80%).

2′-O-(dimethylaminooxyethyl)-5-methyluridine

Triethylamine trihydrofluoride (3.91 mL, 24.0 mmol) was dissolved in dry THF and triethylamine (1.67 mL, 12 mmol, dry, kept over KOH). This mixture of triethylamine-2HF was then added to 5′-O-tert-butyldiphenylsilyl-2′-O-[N,N-dimethylaminooxyethyl]-5-methyluridine (1.40 g, 2.4 mmol) and stirred at room temperature for 24 hrs. Reaction was monitored by TLC (5% MeOH in CH2Cl2). Solvent was removed under vacuum and the residue placed on a flash column and eluted with 10% MeOH in CH₂Cl₂ to get 2′-O-(dimethylaminooxyethyl)-5-methyluridine (766 mg, 92.5%).

5′-O-DMT-2′-O-(dimethylaminooxyethyl)-5-methyluridine

2′-O-(dimethylaminooxyethyl)-5-methyluridine (750 mg, 2.17 mmol) was dried over P205 under high vacuum overnight at 40° C. It was then co-evaporated with anhydrous pyridine (20 mL). The residue obtained was dissolved in pyridine (11 mL) under argon atmosphere. 4-dimethylaminopyridine (26.5 mg, 2.60 mmol), 4,4′-dimethoxytrityl chloride (880 mg, 2.60 mmol) was added to the mixture and the reaction mixture was stirred at room temperature until all of the starting material disappeared. Pyridine was removed under vacuum and the residue chromatographed and eluted with 10% MeOH in CH₂Cl₂ (containing a few drops of pyridine) to get 5′-O-DMT-2′-O-(dimethylamino-oxyethyl)-5-methyluridine (1.13 g, 80%).

5′-O-DMT-2′-O-(2-N,N-dimethylaminooxyethyl)-5-methyluridine-3′-[(2-cyanoethyl)-N,N-diisopropylphosphoramidite]

5′-O-DMT-2′-O-(dimethylaminooxyethyl)-5-methyluridine (1.08 g, 1.67 mmol) was co-evaporated with toluene (20 mL). To the residue N,N-diisopropylamine tetrazonide (0.29 g, 1.67 mmol) was added and dried over P₂O₅ under high vacuum overnight at 40° C. Then the reaction mixture was dissolved in anhydrous acetonitrile (8.4 mL) and 2-cyanoethyl-N,N,N1,N1-tetraisopropylphosphoramidite (2.12 mL, 6.08 mmol) was added. The reaction mixture was stirred at ambient temperature for 4 hrs under inert atmosphere. The progress of the reaction was monitored by TLC (hexane:ethyl acetate 1:1). The solvent was evaporated, then the residue was dissolved in ethyl acetate (70 mL) and washed with 5% aqueous NaHCO₃ (40 mL). Ethyl acetate layer was dried over anhydrous Na₂SO₄ and concentrated. Residue obtained was chromatographed (ethyl acetate as eluent) to get 5′-O-DMT-2′-O-(2-N,N-dimethylaminooxyethyl)-5-methyluridine-3′-[(2-cyanoethyl)-N,N-diisopropylphosphoramidite] as a foam (1.04 g, 74.9%).

2′-(Aminooxyethoxy) nucleoside amidites

2′-(Aminooxyethoxy) nucleoside amidites [also known in the art as 2′-O-(aminooxyethyl) nucleoside amidites] are prepared as described in the following paragraphs. Adenosine, cytidine and thymidine nucleoside amidites are prepared similarly.

N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite]

The 2′-O-aminooxyethyl guanosine analog may be obtained by selective 2′-O-alkylation of diaminopurine riboside. Multigram quantities of diaminopurine riboside may be purchased from Schering AG (Berlin) to provide 2′-O-(2-ethylacetyl) diaminopurine riboside along with a minor amount of the 3′-O-isomer. 2′-O-(2-ethylacetyl) diaminopurine riboside may be resolved and converted to 2′-O-(2-ethylacetyl)guanosine by treatment with adenosine deaminase. (McGee, D. P. C., Cook, P. D., Guinosso, C. J., WO 94/02501 Al 940203.) Standard protection procedures should afford 2′-O-(2-ethylacetyl)-5′-O-(4,4′-dimethoxytrityl)guanosine and 2-N-isobutyryl-6-O-diphenylcarbamoyl-2′-O-(2-ethylacetyl)-5′-O-(4,4′-dimethoxytrityl)guanosine which may be reduced to provide 2-N-isobutyryl-6-O-diphenylcarbamoyl-2′-O-(2-ethylacetyl)-5′-O-(4,4′-dimethoxytrityl) guanosine. As before the hydroxyl group may be displaced by N-hydroxyphthalimide via a Mitsunobu reaction, and the protected nucleoside may phosphitylated as usual to yield 2-N-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite].

Oligonucleotides having methylene(methylimino) (MMI) backbones are synthesized according to U.S. Pat. No. 5,378,825, which is coassigned to the assignee of the present invention and is incorporated herein in its entirety. For ease of synthesis, various nucleoside dimers containing MMI linkages were synthesized and incorporated into oligonucleotides. Other nitrogen-containing backbones are synthesized according to WO 92/20823 which is also coassigned to the assignee of the present invention and incorporated herein in its entirety.

Oligonucleotides having amide backbones are synthesized according to De Mesmaeker et al., Acc. Chem. Res., 28, 366 (1995). The amide moiety is readily accessible by simple and well-known synthetic methods and is compatible with the conditions required for solid phase synthesis of oligonucleotides.

Oligonucleotides with morpholino backbones are synthesized according to U.S. Pat. No. 5,034,506 (Summerton and Weller).

Peptide-nucleic acid (PNA) oligomers are synthesized according to P. E. Nielsen et al., Science, 254, 1497 (1991).

After cleavage from the controlled pore glass column (Applied Biosystems) and deblocking in concentrated ammonium hydroxide at 55° C. for 18 hours, the oligonucleotides are purified by precipitation twice out of 0.5 M NaCl with 2.5 volumes ethanol. Synthesized oligonucleotides were analyzed by polyacrylamide gel electrophoresis on denaturing gels and judged to be at least 85% full length material. The relative amounts of phosphorothioate and phosphodiester linkages obtained in synthesis were periodically checked by ³¹p nuclear magnetic resonance spectroscopy, and for some studies oligonucleotides were purified by HPLC, as described by Chiang et al., J. Biol. Chem., 266, 18162 (1991). Results obtained with HPLC-purified material were similar to those obtained with non-HPLC purified material.

### Example 2

Human mdm2 Oligonucleotide Sequences

The oligonucleotides tested are presented in Table 1. Sequence data are from the cDNA sequence published by Oliner,J. D., et al., Nature, 358, 80 (1992); Genbank accession number Z12020, provided herein as SEQ ID NO: 1. Oligonucleotides were synthesized primarily as chimeric oligonucleotides having a centered deoxy gap of eight nucleotides flanked by 2′-O-methoxyethyl regions.

A549 human lung carcinoma cells (American Type Culture Collection, Manassas, Va.) were routinely passaged at 80-90% confluency in Dulbecco's modified Eagle's medium (DMEM) and 10% fetal bovine serum (Hyclone, Logan, Utah). JEG-3 cells, a human choriocarcinoma cell line (American Type Culture Collection, Manassas, Va.), were maintained in RPMI1640, supplemented with 10% fetal calf serum. All cell culture reagents, except as otherwise indicated, are obtained from Life Technologies (Rockville, Md.).

A549 cells were treated with phosphorothioate oligonucleotides at 200 nM for four hours in the presence of 6 μg/mL LIPOFECTIN™, washed and allowed to recover for an additional 20 hours. Total RNA was extracted and 15-20 μg of each was resolved on 1% gels and transferred to nylon membranes. The blots were probed with a ³²P radiolabeled mdm2 cDNA probe and then stripped and reprobed with a radiolabeled G3PDH probe to confirm equal RNA loading. mdm2 transcripts were examined and quantified with a PhosphorImager (Molecular Dynamics, Sunnyvale, Calif.). Results are shown in Table 2. Oligonucleotides 16506 (SEQ ID NO: 3), 16507 (SEQ ID NO: 4), 16508 (SEQ ID NO: 5), 16510 (SEQ ID NO: 7), 16518 (SEQ ID NO: 15), 16520 (SEQ ID NO: 17), 16521 (SEQ ID NO: 18), 16522 (SEQ ID NO: 19) and 16524 (SEQ ID NO: 21) gave at least approximately 50% of mdm2 mRNA levels. Oligonucleotides 16507 and better than 85% reduction of mdm2.

TABLE 1
Nucleotide Sequences of Human mdm2
Phosphorothioate Oligonucleotides

| | | SEQ | TARGET GENE | GENE |
|---|---|---|---|---|
| ISIS | NUCLEOTIDE SEQUENCE¹ | ID | NUCLEOTIDE | TARGET |
| NO. | (5′->3′) | NO: | CO-ORDINATES² | REGION |
| 16506 | CAGCCAAGCTCGCGCGGTGC | 3 | 0001-0020 | 5′-UTR |
| 16507 | TCTTTCCGACACACAGGGCC | 4 | 0037-0056 | 5′-UTR |
| 16508 | CAGCAGGATCTCGGTCAGAG | 5 | 0095-0114 | 5′-UTR |
| 16509 | GGGCGCTCGTACGCACTAAT | 6 | 0147-0166 | 5′-UTR |
| 16510 | TCGGGGATCATTCCACTCTC | 7 | 0181-0200 | 5′-UTR |
| 16511 | CGGGGTTTTCGCGCTTGGAG | 8 | 0273-0292 | 5′-UTR |
| 16512 | CATTTGCCTGCTCCTCACCA | 9 | 0295-0314 | AUG |
| 16513 | GTATTGCACATTTGCCTGCT | 10 | 0303-0322 | AUG |
| 16514 | AGCACCATCAGTAGGTACAG | 11 | 0331-0350 | ORF |
| 16515 | CTACCAAGTTCCTGTAGATC | 12 | 0617-0636 | ORF |
| 16516 | TCAACTTCAAATTCTACACT | 13 | 1047-1066 | ORF |
| 16517 | TTTACAATCAGGAACATCAA | 14 | 1381-1400 | ORF |
| 16518 | AGCTTCTTTGCACATGTAAA | 15 | 1695-1714 | ORF |
| 16519 | CAGGTCAACTAGGGGAAATA | 16 | 1776-1795 | stop |
| 16520 | TCTTATAGACAGGTCAACTA | 17 | 1785-1804 | stop |
| 16521 | TCCTAGGGTTATATAGTTAG | 18 | 1818-1837 | 3′-UTR |
| 16522 | AAGTATTCACTATTCCACTA | 19 | 1934-1953 | 3′-UTR |
| 16523 | CCAAGATCACCCACTGCACT | 20 | 2132-2151 | 3′-UTR |
| 16524 | AGGTGTGGTGGCAGATGACT | 21 | 2224-2243 | 3′-UTR |
| 16525 | CCTGTCTCTACTAAAAGTAC | 22 | 2256-2275 | 3′-UTR |
| 17604 | ACAAGCCTTCGCTCTACCGG | 23 | scrambled | 16507 |
| | | | control | |
| 17605 | TTCAGCGCATTTGTACATAA | 24 | scrambled | 16518 |
| | | | control | |
| 17615 | TCTTTCCGACACACAGGGCC | 25 | 0037-0056 | 5′-UTR |
| 17616 | AGCTTCTTTGCACATGTAAA | 15 | 1695-1714 | ORF |
| 17755 | CACATGTAAA | 15 | 1695-1714 | ORF |
| 17756 | AGCTTCTTTATACATGTAAA | 26 | 2-base | 17616 |
| | | | mismatch | |
| 17757 | AGCTTCTTTACACATGTAAA | 27 | 1-base | 17616 |
| | | | mismatch | |
| ¹Emboldened residues, 2′-methoxyethoxy-residues (others are 2′-deoxy-) including “C” residues, 5-methyl-cytosines; all linkages are phosphorothioate linkages. | | | | |
| ²Co-ordinates from Genbank Accession No. Z12020, locus name “HSP53ASSG”, SEQ ID NO:1. Oligonucleotides 16505-16511 are targeted to the 5′ UTR of the L-mdm2 transcript as described hereinabove [Landers et al., Cancer Res., 57, 3562 (1997)] Nucleotide coordinates on the Landers sequence [Landers et al., Cancer Res., 57, 3562 (1997) and Genbank accession no. | | | | |
| #U39736] are identical to those shown in Table 1 except for ISIS 16511, which maps to nucleotides 267-286 on the Landers sequence. | | | | |

TABLE 2
Activities of Phosphorothioate Oligonucleotides
Targeted to Human mdm2

| | SEQ | GENE | | |
|---|---|---|---|---|
| | ID | TARGET | % mRNA | % mRNA |
| ISIS No: | NO: | REGION | EXPRESSION | INHIBITION |
| LIPOFECTIN ™ | — | — | 100% | 0% |
| only | | | | |
| 16506 | 3 | 5′-UTR | 45% | 55% |
| 16507 | 4 | 5′-UTR | 13% | 87% |
| 16508 | 5 | 5′-UTR | 38% | 62% |
| 16509 | 6 | 5′-UTR | 161% | — |
| 16510 | 7 | 5′-UTR | 46% | 54% |
| 16511 | 8 | 5′-UTR | 91% | 9% |
| 16512 | 9 | AUG | 89% | 11% |
| 16513 | 10 | AUG | 174% | — |
| 16514 | 11 | Coding | 92% | 8% |
| 16515 | 12 | Coding | 155% | — |
| 16516 | 13 | Coding | 144% | — |
| 16517 | 14 | Coding | 94% | 6% |
| 16518 | 15 | Coding | 8% | 92% |
| 16519 | 16 | stop | 73% | 27% |
| 16520 | 17 | stop | 51% | 49% |
| 16521 | 18 | 3′-UTR | 38% | 62% |
| 16522 | 19 | 3′-UTR | 49% | 51% |
| 16523 | 20 | 3′-UTR | 109% | — |
| 16524 | 21 | 3′-UTR | 47% | 53% |
| 16525 | 22 | 3′-UTR | 100% | — |

### Example 3

Dose Response Of Antisense Oligonucleotide Effects On Human mdm2 mRNA Levels In A549 Cells

Oligonucleotides 16507 and 16518 were tested at different concentrations. A549 cells were grown, treated and processed as described in Example 2. LIPOFECTIN™ was added at a ratio of 3 μg/mL per 100 nM of oligonucleotide. The control included LIPOFECTIN™ at a concentration of 12 μg/mL. Oligonucleotide 17605, an oligonucleotide with different sequence but identical base composition to oligonucleotide 16518, was used as a negative control. Results are shown in Table 3. Oligonucleotides 16507 and 16518 gave approximately 90% inhibition at concentrations greater than 200 nM. No inhibition was seen with oligonucleotide 17605.

TABLE 3
Dose Response of A549 Cells to mdm2
Antisense Oligonucleotides (ASOs)

| | SEQ | | | | |
|---|---|---|---|---|---|
| | ID | ASO Gene | | % mRNA | % mRNA |
| ISIS # | NO: | Target | Dose | Expression | Inhibition |
| control | — | LIPOFECTIN ™ | — | 100% | 0% |
| | | only | | | |
| 16507 | 4 | 5′-UTR | 25 nM | 55% | 45% |
| 16507 | 4 | ″ | 50 nM | 52% | 48% |
| 16507 | 4 | ″ | 100 nM | 24% | 76% |
| 16507 | 4 | ″ | 200 nM | 12% | 88% |
| 16518 | 15 | Coding | 50 nM | 18% | 82% |
| 16518 | 15 | ″ | 100 nM | 14% | 86% |
| 16518 | 15 | ″ | 200 nM | 9% | 91% |
| 16518 | 15 | ″ | 400 nM | 8% | 92% |
| 17605 | 24 | scrambled | 400 nM | 129% | — |
| | | control | | | |

### Example 4

Time Course of Antisense Oligonucleotide Effects on Human mdm2 mRNA Levels in A549 Cells

Oligonucleotides 16507 and 17605 were tested by treating for varying times. A549 cells were grown, treated for times indicated in Table 4 and processed as described in Example 2. Results are shown in Table 4. Oligonucleotide 16507 gave greater than 90% inhibition throughout the time course. No inhibition was seen with oligonucleotide 17605.

TABLE 4
Time Course of Response of Cells to
Human mdm2 Antisense Oligonucleotides (ASOs)

| | SEQ | ASO Gene | | | |
|---|---|---|---|---|---|
| | ID | Target | | % RNA | % RNA |
| ISIS # | NO: | Region | Time | Expression | Inhibition |
| basal | — | LIPOFECTIN ™ | 24 h | 100% | 0% |
| | | only | | | |
| basal | — | LIPOFECTIN ™ | 48 h | 100% | 0% |
| | | only | | | |
| basal | — | LIPOFECTIN ™ | 72 h | 100% | 0% |
| | | only | | | |
| 16518 | 15 | Coding | 24 h | 3% | 97% |
| 16518 | 15 | ″ | 48 h | 6% | 94% |
| 16518 | 15 | ″ | 72 h | 5% | 95% |
| 17605 | 24 | scrambled | 24 h | 195% | — |
| 17605 | 24 | ″ | 48 h | 100% | — |
| 17605 | 24 | ″ | 72 h | 102% | — |

### Example 5

Effect of Antisense Oligonucleotides on Cell Proliferation in A549 Cells

A549 cells were treated on day 0 for four hours with 400 nM oligonucleotide and 12 mg/mL LIPOFECTIN. After four hours, the medium was replaced. Twenty-four, forty-eight or seventy-two hours after initiation of oligonucleotide treatment, live cells were counted on a hemacytometer. Results are shown in Table 5.

TABLE 5
Antisense Inhibition of Cell Proliferation
in A549 cells

| | SEQ | | | | |
|---|---|---|---|---|---|
| | ID | ASO Gene | | | % Growth |
| ISIS # | NO: | Target Region | Time | % Cell Growth | Inhibition |
| basal | — | LIPOFECTIN ™ | 24 h | 100% | 0% |
| | | only | | | |
| basal | — | LIPOFECTIN ™ | 48 h | 100% | 0% |
| | | only | | | |
| basal | — | LIPOFECTIN ™ | 72 h | 100% | 0% |
| | | only | | | |
| 16518 | 15 | Coding | 24 h | 53% | 47% |
| 16518 | 15 | ″ | 48 h | 27% | 73% |
| 16518 | 15 | ″ | 72 h | 17% | 83% |
| 17605 | 24 | scrambled | 24 h | 93% | 7% |
| 17605 | 24 | ″ | 48 h | 76% | 24% |
| 17605 | 24 | ″ | 72 h | 95% | 5% |

### Example 6

Effect of mdm2 Antisense Oligonucleotide on p53 Protein Levels

JEG3 cells were cultured and treated as described in Example 2, except that 300 nM oligonucleotide and 9 μg/mL of LIPOFECTIN™ was used.

For determination of p53 protein levels by western blot, cellular extracts were prepared using 300 ul of RIPA extraction buffer per 100-mm dish. The protein concentration was quantified by Bradford assay using the BioRad kit (BioRad, Hercules, Calif.). Equal amounts of protein were loaded on 10% or 12% SDS-PAGE mini-gel (Novex, San Diego, Calif.). Once transferred to PVDF membranes (Millipore, Bedford, Mass.), the membranes were then treated for a minimum of 2 h with specific primary antibody (p53 antibody, Transduction Laboratories, Lexington, Ky.) followed by incubation with secondary antibody conjugated to HRP. The results were visualized by ECL Plus Western Blotting Detection System (Amersham Pharmacia Biotech, Piscataway, N.J.). In some experiments, the blots were stripped in stripping buffer (2% SDS, 12.5 mM Tris, pH 6.8) for 30 min. at 50° C. After extensive washing, the blots were blocked and blotted with different primary antibody.

Results are shown in Table 6. Treatment with mdm2 antisense oligonucleotide results in the induction of p53 levels. An approximately three-fold increase in activity was seen under these conditions.

TABLE 6
Activity of ISIS 16518 on p53 Protein Levels

| | SEQ ID | GENE TARGET | % protein |
|---|---|---|---|
| ISIS No: | NO: | REGION | EXPRESSION |
| LIPOFECTIN ™ | — | — | 100% |
| only | | | |
| 16518 | 15 | coding | 289% |

### Example 7

Effect of ISIS 16518 on Expression of p53 Mediated Genes

p53 is known to regulate the expression of a number of genes and to be involved in apoptosis. Representative genes known to be regulated by p53 include p21 (Deng, C., et al., Cell, 1995, 82, 675), bax (Selvakumaran, M., et al., Oncogene, 1994, 9, 1791-1798) and GADD45 (Carrier, F., et al., J. Biol. Chem., 1994, 269, 32672-32677). The effect of an mdm2 antisense oligonucleotide on these genes is investigated by RPA analysis using the RIBOQUANT™ RPA kit, according to the manufacturer's instructions (Pharmingen, San Diego, Calif.), along with the hSTRESS-1 multi-probe template set. Included in this template set are bclx, p53, GADD45, c-fos, p21, bax, bcl2 and mcl1. The effect of mdm2 antisense oligonucleotides on p53-mediated apoptosis can readily be assessed using commercial kits based on apoptotic markers such as DNA fragmentation or caspase activity.

### Example 8

Additional Human mdm2 Chimeric (deoxy gapped) Antisense Oligonucleotides

Additional oligonucleotides targeted to the 5′-untranslated region of human mdm2 mRNA were designed and synthesized. Sequence data are from the cDNA sequence published by Zauberman, A., et al., Nucleic Acids Res., 23, 2584 (1995); Genbank accession number HSU28935. Oligonucleotides were synthesized primarily as chimeric oligonucleotides having a centered deoxy gap of eight nucleotides flanked by 2′-O-methoxyethyl regions. The oligonucleotide sequences are shown in Table 7. These oligonucleotides were tested in A549 cells as described in Example 2. Results are shown in Table 8.

TABLE 7
Nucleotide Sequences of additional Human mdm2
Chimeric (deoxy gapped) Phosphorothioate
Oligonucleotides

| | | SEQ | TARGET GENE | GENE |
|---|---|---|---|---|
| ISIS | NUCLEOTIDE SEQUENCE¹ | ID | NUCLEOTIDE | TARGET |
| NO. | (5′->3′) | NO: | CO-ORDINATES² | REGION |
| 21926 | CTACCCTCCAATCGCCACTG | 28 | 0238-0257 | coding |
| 21927 | GGTCTACCCTCCAATCGCCA | 29 | 0241-0260 | coding |
| 21928 | CGTGCCCACAGGTCTACCCT | 30 | 0251-0270 | coding |
| 21929 | AAGTGGCGTGCGTCCGTGCC | 31 | 0265-0284 | coding |
| 21930 | AAAGTGGCGTGCGTCCGTGC | 32 | 0266-0285 | coding |

¹Emboldened residues, 2′-methoxyethoxy-residues (others are 2′-deoxy-); all 2′-methoxyethoxy-cytosine and 2′-deoxy-cytosine residues, 5-methyl-cytosines; all linkages are phosphorothioate linkages. ²Co-ordinates from Genbank Accession No. U28935, locus name “HSU28935”, SEQ ID NO:2.

TABLE 8
Activities of Chimeric (deoxy gapped) Oligonucleotides
Targeted to Human mdm2

| | SEQ | GENE | | |
|---|---|---|---|---|
| | ID | TARGET | % mRNA | % mRNA |
| ISIS No: | NO: | REGION | EXPRESSION | INHIBITION |
| LIPOFECTIN ™ | — | — | 100% | 0% |
| only | | | | |
| 21926 | 28 | coding | 345% | — |
| 21927 | 29 | coding | 500% | — |
| 21928 | 30 | coding | 417% | — |
| 21929 | 31 | coding | 61% | 39% |
| 21930 | 32 | coding | 69% | 31% |

These oligonucleotide sequences were also tested for their ability to reduce mdm2 protein levels. JEG3 cells were cultured and treated as described in Example 2, except that 300 nM oligonucleotide and 9 μg/mL of LIPOFECTIN™ was used. Mdm2 protein levels were assayed by Western blotting as described in Example 6, except a mouse anti-mdm2 monoclonal antibody (Santa Cruz Biotechnology, Santa Cruz, Calif.) was used. Results are shown in Table 9.

TABLE 9
Activities of Chimeric (deoxy gapped)
Human mdm2 Antisense Oligonucleotides
on mdm2 Protein Levels

| | SEQ | GENE | | |
|---|---|---|---|---|
| | ID | TARGET | % PROTEIN | % PROTEIN |
| ISIS No: | NO: | REGION | EXPRESSION | INHIBITION |
| LIPOFECTIN ™ | — | — | 100% | 0% |
| only | | | | |
| 21926 | 28 | coding | 30% | 70% |
| 21927 | 29 | coding | 18% | 82% |
| 21928 | 30 | coding | 43% | 57% |
| 21929 | 31 | coding | 62% | 38% |
| 21930 | 32 | coding | 56% | 44% |

Each oligonucleotide tested reduced mdm2 protein levels by greater than approximately 40%. Maximum inhibition was seen with oligonucleotide 21927 (SEQ ID NO. 29) which gave greater than 80% inhibition of mdm2 protein.

### Example 9

Additional Human mdm2 Antisense Oligonucleotides

Additional oligonucleotides targeted to human mdm2 mRNA were signed and synthesized. Sequence data are from the cDNA sequence published by Zauberman, A., et al., Nucleic Acids Res., 23, 2584 (1995); Genbank accession number HSU28935. Oligonucleotides were synthesized in 96 well plate format via solid phase P(III) phosphoramidite chemistry on an automated synthesizer capable of assembling 96 sequences simultaneously in a standard 96 well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-di-isopropyl phosphoramidites were purchased from commercial vendors (e.g. PE-Applied Biosystems, Foster City, Calif., or Pharmacia, Piscataway, N.J.). Non-standard nucleosides are synthesized as per published methods. They are utilized as base protected beta-cyanoethyldiisopropyl phosphoramidites.

Oligonucleotides were cleaved from support and deprotected with concentrated NH₄0H at elevated temperature (55-60° C.) for 12-16 hours and the released product then dried in vacuo. The dried product was then re-suspended in sterile water to afford a master plate from which all analytical and test plate samples are then diluted utilizing robotic pipettors.

Two sets of oligonucleotides were synthesized; one as phosphorothioate oligodeoxynucleotides, the other as chimeric oligonucleotides having a centered deoxy gap of ten nucleotides flanked by regions of five 2′-O-methoxyethyl nucleotides. These oligonucleotides sequences are shown in Tables 10 and 11.

mRNA was isolated using the RNAEASY™ kit (Qiagen, Santa Clarita, Calif.).

TABLE 10
Nucleotide Sequences of Human mdm2
Phosphorothioate Oligodeoxynucleotides

| | | SEQ | TARGET GENE | GENE |
|---|---|---|---|---|
| ISIS | NUCLEOTIDE SEQUENCE¹ | ID | NUCLEOTIDE | TARGET |
| NO. | (5′->3′) | NO: | CO-ORDINATES² | REGION |
| 31393 | CAGCCAAGCTCGCGCGGTGC | 3 | 0001-0020 | 5′ UTR |
| 31712 | AAGCAGCCAAGCTCGCGCGG | 33 | 0004-0023 | 5′ UTR |
| 31552 | CAGGCCCCAGAAGCAGCCAA | 34 | 0014-0033 | 5′ UTR |
| 31713 | GCCACACAGGCCCCAGAAGC | 35 | 0020-0039 | 5′ UTR |
| 31394 | ACACACAGGGCCACACAGGC | 36 | 0029-0048 | 5′ UTR |
| 31714 | TTCCGACACACAGGGCCACA | 37 | 0034-0053 | 5′ UTR |
| 31553 | GCTCCATCTTTCCGACACAC | 38 | 0043-0062 | 5′ UTR |
| 31715 | GCTTCTTGCTCCATCTTTCC | 39 | 0050-0069 | 5′ UTR |
| 31395 | CCCTCGGGCTCGGCTTCTTG | 40 | 0062-0081 | 5′ UTR |
| 31716 | GCGGCCGCCCCTCGGGCTCG | 41 | 0070-0089 | 5′ UTR |
| 31554 | AAGCAGCAGGATCTCGGTCA | 42 | 0098-0107 | 5′ UTR |
| 31717 | GCTGCGAAAGCAGCAGGATC | 43 | 0105-0124 | 5′ UTR |
| 31396 | TGCTCCTGGCTGCGAAAGCA | 44 | 0113-0132 | 5′ UTR |
| 31718 | GGGACGGTGCTCCTGGCTGC | 45 | 0120-0139 | 5′ UTR |
| 31555 | ACTGGGCGCTCGTACGCACT | 46 | 0150-0169 | 5′ UTR |
| 31719 | GCCAGGGCACTGGGCGCTCG | 47 | 0158-0177 | 5′ UTR |
| 31397 | TCTCCGGGCCAGGGCACTGG | 48 | 0165-0184 | 5′ UTR |
| 31720 | TCATTCCACTCTCCGGGCCA | 49 | 0174-0193 | 5′ UTR |
| 31556 | GGAAGCACGACGCCCTGGGC | 50 | 0202-0221 | 5′ UTR |
| 31721 | TACTGCGGAAGCACGACGCC | 51 | 0208-0227 | 5′ UTR |
| 31398 | GGGACTGACTACTGCGGAAG | 52 | 0217-0236 | 5′ UTR |
| 31722 | TCAAGACTCCCCAGTTTCCT | 53 | 0242-0261 | 5′ UTR |
| 31557 | CCTGCTCCTCACCATCCGGG | 54 | 0289-0308 | 5′ UTR |
| 31399 | TTTGCCTGCTCCTCACCATC | 55 | 0293-0312 | AUG |
| 31400 | ATTTGCCTGCTCCTCACCAT | 56 | 0294-0313 | AUG |
| 31401 | CATTTGCCTGCTCCTCACCA | 9 | 0295-0314 | AUG |
| 31402 | ACATTTGCCTGCTCCTCACC | 57 | 0296-0315 | AUG |
| 31403 | CACATTTGCCTGCTCCTCAC | 58 | 0297-0316 | AUG |
| 31404 | GCACATTTGCCTGCTCCTCA | 59 | 0298-0317 | AUG |
| 31405 | TGCACATTTGCCTGCTCCTC | 60 | 0299-0318 | AUG |
| 31406 | TTGCACATTTGCCTGCTCCT | 61 | 0300-0319 | AUG |
| 31407 | ATTGCACATTTGCCTGCTCC | 62 | 0301-0320 | AUG |
| 31408 | TATTGCACATTTGCCTGCTC | 63 | 0302-0321 | AUG |
| 31409 | GTATTGCACATTTGCCTGCT | 10 | 0303-0322 | AUG |
| 31410 | GGTATTGCACATTTGCCTGC | 64 | 0304-0323 | AUG |
| 31411 | TGGTATTGCACATTTGCCTG | 65 | 0305-0324 | AUG |
| 31412 | TTGGTATTGCACATTTGCCT | 66 | 0306-0325 | AUG |
| 31413 | GTTGGTATTGCACATTTGCC | 67 | 0307-0326 | AUG |
| 31414 | TGTTGGTATTGCACATTTGC | 68 | 0308-0327 | AUG |
| 31415 | ATGTTGGTATTGCACATTTG | 69 | 0309-0328 | AUG |
| 31416 | CATGTTGGTATTGCACATTT | 70 | 0310-0329 | AUG |
| 31417 | ACATGTTGGTATTGCACATT | 71 | 0311-0330 | AUG |
| 31418 | GACATGTTGGTATTGCACAT | 72 | 0312-0331 | AUG |
| 31419 | AGACATGTTGGTATTGCACA | 73 | 0313-0332 | AUG |
| 31420 | CAGACATGTTGGTATTGCAC | 74 | 0314-0333 | AUG |
| 31558 | CAGTAGGTACAGACATGTTG | 75 | 0323-0342 | coding |
| 31723 | TACAGCACCATCAGTAGGTA | 76 | 0334-0353 | coding |
| 31421 | GGAATCTGTGAGGTGGTTAC | 77 | 0351-0370 | coding |
| 31559 | TTCCGAAGCTGGAATCTGTG | 78 | 0361-0380 | coding |
| 31724 | AGGGTCTCTTGTTCCGAAGC | 79 | 0372-0391 | coding |
| 31422 | GCTTTGGTCTAACCAGGGTC | 80 | 0386-0405 | coding |
| 31560 | GCAATGGCTTTGGTCTAACC | 81 | 0392-0411 | coding |
| 31725 | TAACTTCAAAAGCAATGGCT | 82 | 0403-0422 | coding |
| 31423 | GTGCACCAACAGACTTTAAT | 83 | 0422-0441 | coding |
| 31561 | ACCTCTTTCATAGTATAAGT | 84 | 0450-0469 | coding |
| 31726 | ATAATATACTGGCCAGGATA | 85 | 0477-0496 | coding |
| 31424 | TAATCGTTTAGTCATAATAT | 86 | 0490-0509 | coding |
| 31727 | ATCATATAATCGTTTAGTCA | 87 | 0496-0515 | coding |
| 31562 | GCTTCTCATCATATAATCGT | 38 | 0503-0522 | coding |
| 31728 | CAATATGTTGTTGCTTCTCA | 89 | 0515-0534 | coding |
| 31425 | GAACAATATACAATATGTTG | 90 | 0525-0544 | coding |
| 31729 | TCATTTGAACAATATACAAT | 91 | 0531-0550 | coding |
| 31563 | TAGAAGATCATTTGAACAAT | 92 | 0538-0557 | coding |
| 31730 | AACAAATCTCCTAGAAGATC | 93 | 0549-0568 | coding |
| 31426 | TGGCACGCCAAACAAATCTC | 94 | 0559-0578 | coding |
| 31731 | AGAAGCTTGGCACGCCAAAC | 95 | 0566-0585 | coding |
| 31564 | CTTTCACAGAGAAGCTTGGC | 96 | 0575-0594 | coding |
| 31732 | TTTTCCTGTGCTCTTTCACA | 97 | 0587-0606 | coding |
| 31427 | TATATATTTTCCTGTGCTCT | 98 | 0593-0612 | coding |
| 31733 | ATCATGGTATATATTTTCCT | 99 | 0600-0619 | coding |
| 31565 | TTCCTGTAGATCATGGTATA | 100 | 0609-0628 | coding |
| 31734 | TACTACCAAGTTCCTGTAGA | 101 | 0619-0638 | coding |
| 31428 | TTCCTGCTGATTGACTACTA | 102 | 0634-0653 | coding |
| 31566 | TGAGTCCGATGATTCCTGCT | 103 | 0646-0665 | coding |
| 31735 | CAGATGTACCTGAGTCCGAT | 104 | 0656-0675 | coding |
| 31429 | CTGTTCTCACTCACAGATGT | 105 | 0669-0688 | coding |
| 31567 | TTCAAGGTGACACCTGTTCT | 106 | 0632-0701 | coding |
| 31736 | ACTCCCACCTTCAAGGTGAC | 107 | 0691-0710 | coding |
| 31430 | GGTCCTTTTGATCACTCCCA | 108 | 0704-0723 | coding |
| 31568 | AAGCTCTTGTACAAGGTCCT | 109 | 0718-0737 | coding |
| 31737 | CTCTTCCTGAAGCTCTTGTA | 110 | 0727-0746 | coding |
| 31431 | AAGATGAAGGTTTCTCTTCC | 111 | 0740-0759 | coding |
| 31569 | AAACCAAATGTGAAGATGAA | 112 | 0752-0771 | coding |
| 31738 | ATGGTCTAGAAACCAAATGT | 113 | 0761-0780 | coding |
| 31432 | CTAGATGAGGTAGATGGTCT | 114 | 0774-0793 | coding |
| 31570 | AATTGCTCTCCTTCTAGATG | 115 | 0787-0806 | coding |
| 31739 | TCTGTCTCACTAATTGCTCT | 116 | 0798-0817 | coding |
| 31433 | TCTGAATTTTCTTCTGTCTC | 117 | 0810-0829 | coding |
| 31571 | CACCAGATAATTCATCTGAA | 118 | 0824-0843 | coding |
| 31740 | TTTGTCGTTCACCAGATAAT | 119 | 0833-0852 | coding |
| 31434 | GTGGCGTTTTCTTTGTCGTT | 120 | 0844-0863 | coding |
| 31572 | TACTATCAGATTTGTGGCGT | 121 | 0857-0876 | coding |
| 31741 | GAAAGGGAAATACTATCAGA | 122 | 0867-0886 | coding |
| 31435 | GCTTTCATCAAAGGAAAGGG | 123 | 0880-0899 | coding |
| 31573 | TACACACAGAGCCAGGCTTT | 124 | 0895-0914 | coding |
| 31742 | CTCCCTTATTACACACAGAG | 125 | 0904-0923 | coding |
| 31436 | TCACAACATATCTCCCTTAT | 126 | 0915-0934 | coding |
| 31574 | CTACTGCTTCTTTCACAACA | 127 | 0927-0946 | coding |
| 31743 | GATTCACTGCTACTGCTTCT | 128 | 0936-0955 | coding |
| 31437 | TGGCGTCCCTGTAGATTCAC | 129 | 0949-0968 | coding |
| 31575 | AAGATCCGGATTCGATGGCG | 130 | 0964-0983 | coding |
| 31744 | CAGCATCAAGATCCGGATTC | 131 | 0971-0990 | coding |
| 31438 | GTTCACTTACACCAGCATCA | 132 | 0983-1002 | coding |
| 31576 | CAATCACCTGAATGTTCACT | 133 | 0996-1015 | coding |
| 31745 | CTGATCCAACCAATCACCTG | 134 | 1006-1025 | coding |
| 31439 | GAAACTGAATCCTGATCCAA | 135 | 1017-1036 | coding |
| 31746 | TGATCTGAAACTGAATCCTG | 136 | 1023-1042 | coding |
| 31577 | CTACACTAAACTGATCTGAA | 137 | 1034-1053 | coding |
| 31747 | CAACTTCAAATTCTACACTA | 138 | 1046-1065 | coding |
| 31440 | AGATTCAACTTCAAATTCTA | 139 | 1051-1070 | coding |
| 31748 | GAGTCGAGAGATTCAACTTC | 140 | 1059-1078 | coding |
| 31578 | TAATCTTCTGAGTCGAGAGA | 141 | 1068-1087 | coding |
| 31749 | CTAAGGCTATAATCTTCTGA | 142 | 1077-1096 | coding |
| 31441 | TTCTTCACTAAGGCTATAAT | 143 | 1084-1103 | coding |
| 31750 | TCTTGTCCTTCTTCACTAAG | 144 | 1092-1111 | coding |
| 31579 | CTCAGAGTTCTTGTCCTTCT | 145 | 1100-1119 | coding |
| 31751 | TTCATCTGAGAGTTCTTGTC | 146 | 1105-1124 | coding |
| 31442 | CCTCATCATCTTCATCTGAG | 147 | 1115-1134 | coding |
| 31752 | CTTGATATACCTCATCATCT | 148 | 1124-1143 | coding |
| 31753 | ATACACAGTAACTTGATATA | 149 | 1135-1154 | coding |
| 31443 | CTCTCCCCTGCCTGATACAC | 150 | 1149-1168 | coding |
| 31580 | GAATCTGTATCACTCTCCCC | 151 | 1161-1180 | coding |
| 31754 | TCTTCAAATCAATCTGTATC | 152 | 1170-1189 | coding |
| 31444 | AAATTTCAGGATCTTCTTCA | 153 | 1184-1203 | coding |
| 31581 | AGTCAGCTAAGGAAATTTCA | 154 | 1196-1215 | coding |
| 31755 | GCATTTCCAATAGTCAGCTA | 155 | 1207-1226 | coding |
| 31445 | CATTGCATGAAGTGCATTTC | 156 | 1220-1239 | coding |
| 31756 | TCATTTCATTGCATGAAGTG | 157 | 1226-1245 | coding |
| 31582 | CATCTGTTGCAATGTGATGG | 158 | 1257-1276 | coding |
| 31757 | GAAGGGCCCAACATCTGTTG | 159 | 1268-1287 | coding |
| 31446 | TTCTCACGAAGGGCCCAACA | 160 | 1275-1294 | coding |
| 31758 | GAAGCCAATTCTCACGAAGG | 161 | 1283-1302 | coding |
| 31583 | TATCTTCAGGAAGCCAATTC | 162 | 1292-1311 | coding |
| 31759 | CTTTCCCTTTATCTTCAGGA | 163 | 1301-1320 | coding |
| 31447 | TCCCCTTTATCTTTCCCTTT | 164 | 1311-1330 | coding |
| 31584 | CTTTCTCAGAGATTTCCCCT | 165 | 1325-1344 | coding |
| 31760 | CAGTTTGGCTTTCTCAGAGA | 166 | 1333-1352 | coding |
| 31448 | GTGTTGAGTTTTCCAGTTTG | 167 | 1346-1365 | coding |
| 31585 | CCTCTTCAGCTTGTGTTGAG | 168 | 1358-1377 | coding |
| 31761 | ACATCAAAGCCCTCTTCAGC | 169 | 1368-1787 | coding |
| 31449 | GAATCATTCACTATAGTTTT | 170 | 1401-1420 | coding |
| 31586 | ATGACTCTCTGGAATCATTC | 171 | 1412-1431 | coding |
| 31762 | CCTCAACACATGACTCTCTG | 172 | 1421-1440 | coding |
| 31450 | TTATCATCATTTTCCTCAAC | 173 | 1434-1453 | coding |
| 31763 | TAATTTTATCATCATTTTCC | 174 | 1439-1458 | coding |
| 31587 | GAAGCTTGTGTAATTTTATC | 175 | 1449-1468 | coding |
| 31764 | TGATTGTGAAGCTTGTGTAA | 176 | 1456-1475 | coding |
| 31451 | CACTTTCTTGTGATTGTGAA | 177 | 1466-1485 | coding |
| 31588 | GCTGAGAATAGTCTTCACTT | 178 | 1481-1500 | coding |
| 31765 | AGTTGATGGCTGAGAATAGT | 179 | 1489-1508 | coding |
| 31452 | TGCTACTAGAAGTTGATGGC | 180 | 1499-1518 | coding |
| 31766 | TAAATAATGCTACTAGAAGT | 181 | 1506-1525 | coding |
| 31589 | CTTGGCTGCTATAAATAATG | 182 | 1517-1536 | coding |
| 31590 | ATCTTCTTGGCTGCTATAAA | 183 | 1522-1541 | coding |
| 31453 | AACTCTTTCACATCTTCTTG | 184 | 1533-1552 | coding |
| 31767 | CCCTTTCAAACTCTTTCACA | 185 | 1541-1560 | coding |
| 31591 | GGGTTTCTTCCCTTTCAAAC | 186 | 1550-1569 | coding |
| 31768 | TCTTTGTCTTGGGTTTCTTC | 187 | 1560-1579 | coding |
| 31454 | CTCTCTTCTTTGTCTTGGGT | 188 | 1566-1585 | coding |
| 31592 | AACTAGATTCCACACTCTCT | 189 | 1580-1599 | coding |
| 31769 | CAAGGTTCAATGGCATTAAG | 190 | 1605-1624 | coding |
| 31455 | TGACAAATCACACAAGGTTC | 191 | 1617-1636 | coding |
| 31593 | TCCACCTTCACAAATCACAC | 192 | 1624-1643 | coding |
| 31594 | ATGGACAATGCAACCATTTT | 193 | 1648-1667 | coding |
| 31770 | TGTTTTGCCATGGACAATGC | 194 | 1657-1676 | coding |
| 31456 | TAAGATGTCCTGTTTTGCCA | 195 | 1667-1686 | coding |
| 31595 | GCAGGCCATAAGATGTCCTG | 196 | 1675-1694 | coding |
| 31596 | ACATGTAAAGCAGGCCATAA | 197 | 1684-1703 | coding |
| 31771 | CTTTGCACATGTAAAGCAGG | 198 | 1690-1709 | coding |
| 31457 | TTTCTTTAGCTTCTTTGCAC | 199 | 1702-1721 | coding |
| 31597 | TTATTCCTTTTCTTTAGCTT | 200 | 1710-1729 | coding |
| 31598 | TGGGCAGGGCTTATTCCTTT | 201 | 1720-1739 | coding |
| 31772 | ACATACTGGGCAGGGCTTAT | 202 | 1726-1745 | coding |
| 31458 | TTGGTTGTCTACATACTGGG | 203 | 1736-1755 | coding |
| 31599 | TCATTTGAATTGGTTGTCTA | 204 | 1745-1764 | coding |
| 31600 | AAGTTAGCACAATCATTTGA | 205 | 1757-1776 | coding |
| 31601 | TCTCTTATAGACAGGTCAAC | 206 | 1787-1806 | STOP |
| 31459 | AAATATATAATTCTCTTATA | 207 | 1798-1817 | 3′ UTR |
| 31602 | AGTTAGAAATATATAATTCT | 208 | 1804-1823 | 3′ UTR |
| 31773 | ATATAGTTAGAAATATATAA | 209 | 1808-1827 | 3′ UTR |
| 31603 | CTAGGGTTATATAGTTAGAA | 210 | 1816-1835 | 3′ UTR |
| 31774 | TAAATTCCTAGGGTTATATA | 211 | 1823-1842 | 3′ UTR |
| 31460 | CAGGTTGTCTAAATTCCTAG | 212 | 1832-1851 | 3′ UTR |
| 31604 | ATAAATTTCAGGTTGTCTAA | 213 | 1840-1859 | 3′ UTR |
| 31605 | ATATATGTGAATAAATTTCA | 214 | 1850-1869 | 3′ UTR |
| 31606 | CTTTGATATATGTGAATAAA | 215 | 1855-1874 | 3′ UTR |
| 31461 | CATTTTCTCACTTTGATATA | 216 | 1865-1884 | 3′ UTR |
| 31607 | ATTGAGGCATTTTCTCACTT | 217 | 1872-1891 | 3′ UTR |
| 31608 | AATCTATGTGAATTGAGGCA | 218 | 1883-1902 | 3′ UTR |
| 31609 | AGAAGAAATCTATGTGACTT | 219 | 1889-1908 | 3′ UTR |
| 31462 | ATACTAAAGAGAAGAAATCT | 220 | 1898-1917 | 3′ UTR |
| 31610 | GTCAATTATACTAAAGAGAA | 221 | 1905-1924 | 3′ UTR |
| 31775 | TAGGTCAATTATACTAAAGA | 222 | 1908-1927 | 3′ UTR |
| 31611 | CAAAGTAGGTCAATTATACT | 223 | 1913-1932 | 3′ UTR |
| 31776 | CCACTACCAAAGTAGGTCAA | 224 | 1920-1939 | 3′ UTR |
| 31463 | AGTATTCACTATTCCACTAC | 225 | 1933-1952 | 3′ UTR |
| 31612 | TATAGTAAGTATTCACTATT | 226 | 1940-1959 | 3′ UTR |
| 31613 | AGTCAAATTATAGTAAGTAT | 227 | 1948-1967 | 3′ UTR |
| 31777 | CATATTCAAGTCAAATTATA | 228 | 1956-1975 | 3′ UTR |
| 31464 | AAACGATGAGCTACATATTC | 229 | 1969-1988 | 3′ UTR |
| 31778 | GTGTAAAGGATGAGCTACAT | 230 | 1973-1992 | 3′ UTR |
| 31614 | TAGGAGTTGGTGTAAAGGAT | 231 | 1982-2001 | 3′ UTR |
| 31779 | TTTAAAATTAGGAGTTGGTG | 232 | 1990-2009 | 3′ UTR |
| 31615 | GAAATTATTTAAAATTAGGA | 233 | 1997-2016 | 3′ UTR |
| 31465 | CAGAGTAGAAATTATTTAAA | 234 | 2004-2023 | 3′ UTR |
| 31616 | CTCATTTAAGACAGAGTAGA | 235 | 2015-2034 | 3′ UTR |
| 31780 | TACTTCTCATTTAAGACAGA | 236 | 2020-2039 | 3′ UTR |
| 31617 | CATATACATATTTAAGAAAA | 237 | 2051-2070 | 3′ UTR |
| 31466 | TTAAATGTCATATACATATT | 238 | 2059-2078 | 3′ UTR |
| 31618 | TAATAAGTTACATTTAAATG | 239 | 2072-2091 | 3′ UTR |
| 31619 | GTAACAGAGCAAGACTCGGT | 240 | 2103-2122 | 3′ UTR |
| 31467 | CAGCCTGGGTAACAGAGCAA | 241 | 2111-2130 | 3′ UTR |
| 31781 | CACTCCAGCCTGGGTAACAG | 242 | 2116-2135 | 3′ UTR |
| 31620 | CCCACTGCACTCCAGCCTGG | 243 | 2123-2142 | 3′ UTR |
| 31782 | GCCAAGATCACCCACTGCAC | 244 | 2133-2152 | 3′ UTR |
| 31621 | GCAGTGAGCCAAGATCACCC | 245 | 2140-2159 | 3′ UTR |
| 31468 | GAGCTTGCAGTGAGCCAAGA | 246 | 2146-2165 | 3′ UTR |
| 31783 | GAGGGCAGAGCTTGCAGTGA | 247 | 2153-2172 | 3′ UTR |
| 31622 | CAGGAGAATGGTGCGAACCC | 248 | 2176-2195 | 3′ UTR |
| 31623 | AGGCTGAGGCAGGAGAATGG | 249 | 2185-2204 | 3′ UTR |
| 31784 | ATTGGGAGGCTGAGGCAGGA | 250 | 2191-2210 | 3′ UTR |
| 31469 | CAAGCTAATTGGGAGGCTGA | 251 | 2198-2217 | 3′ UTR |
| 31624 | AGGCCAAGCTAATTGGGAGG | 252 | 2202-2221 | 3′ UTR |
| 31785 | ATGACTGTAGGCCAAGCTAA | 253 | 2210-2229 | 3′ UTR |
| 31625 | CAGATGACTGTAGGCCAAGC | 254 | 2213-2232 | 3′ UTR |
| 31786 | GGTGGCAGATGACTGTAGGC | 255 | 2218-2237 | 3′ UTR |
| 31626 | AGGTGTGGTGGCAGATGACT | 21 | 2224-2243 | 3′ UTR |
| 31470 | AATTAGCCAGGTGTGGTGGC | 256 | 2232-2251 | 3′ UTR |
| 31627 | GTCTCTACTAAAAGTACAAA | 257 | 2253-2272 | 3′ UTR |
| 31628 | CGGTGAAACCCTGTCTCTAC | 258 | 2265-2284 | 3′ UTR |
| 31787 | TGGCTAACACGGTGAAACCC | 259 | 2274-2293 | 3′ UTR |
| 31471 | AGACCATCCTGGCTAACACG | 260 | 2283-2302 | 3′ UTR |
| 31788 | GAGATCGAGACCATCCTGGC | 261 | 2290-2309 | 3′ UTR |
| 31629 | GAGGTCAGGAGATCGAGACC | 262 | 2298-2317 | 3′ UTR |
| 31789 | GCGGATCACGAGGTCAGGAG | 263 | 2307-2326 | 3′ UTR |
| 31472 | AGGCCGAGGTGGGCGGATCA | 264 | 2319-2338 | 3′ UTR |
| 31790 | TTTGGGAGGCCGAGGTGGGC | 265 | 2325-2344 | 3′ UTR |
| 31630 | TCCCAGCACTTTGGGAGGCC | 266 | 2334-2353 | 3′ UTR |
| 31791 | CCTGTAATCCCAGCACTTTG | 267 | 2341-2360 | 3′ UTR |
| 31631 | GTGGCTCATGCCTGTAATCC | 268 | 2351-2370 | 3′ UTR |

¹All deoxy cytosines residues are 5-methyl-cytosines; all linkages are phosphorothioate linkages.
²Co-ordinates from Genbank Accession No. Z12020, locus name “H5P53ASSG”, SEQ ID NO:1.

TABLE 11
Nucleotide Sequences of Human mdm2
Chimeric (deoxy gapped) Oligonucleotides

| | | SEQ | TARGET GENE | GENE |
|---|---|---|---|---|
| ISIS | NUCLEOTIDE SEQUENCE¹ | ID | NUCLEOTIDE | TARGET |
| NO. | (5′->3′) | NO: | CO-ORDINATES² | REGION |
| 31393 | CAGCCAAGCTCGCGCGGTGC | 3 | 0001-0020 | 5′ UTR |
| 31712 | AAGCAGCCAAGCTCGCGCGG | 33 | 0004-0023 | 5′ UTR |
| 31552 | CAGGCCCCAGAAGCAGCCAA | 34 | 0014-0033 | 5′ UTR |
| 31713 | GCCACACAGGCCCCAGAAGC | 35 | 0020-0039 | 5′ UTR |
| 31394 | ACACACAGGGCCACACAGGC | 36 | 0029-0048 | 5′ UTR |
| 31714 | TTCCGACACACAGGGCCACA | 37 | 0034-0053 | 5′ UTR |
| 31553 | GCTCCATCTTTCCGACACAC | 38 | 0043-0062 | 5′ UTR |
| 31715 | GCTTCTTGCTCCATCTTTCC | 39 | 0050-0069 | 5′ UTR |
| 31395 | CCCTCGGGCTCGGCTTCTTG | 40 | 0062-0081 | 5′ UTR |
| 31716 | GCGCCCGCCCCTCGGGCTCG | 41 | 0070-0089 | 5′ UTR |
| 31554 | AAGCAGCAGGATCTCGGTCA | 42 | 0098-0107 | 5′ UTR |
| 31717 | GCTGCGAAAGCAGCAGGATC | 43 | 0105-0124 | 5′ UTR |
| 31396 | TGCTCCTGGCTGCGAAAGCA | 44 | 0113-0132 | 5′ UTR |
| 31718 | GGGACGGTGCTCCTGGCTGC | 45 | 0120-0139 | 5′ UTR |
| 31555 | ACTGGGCGCTCGTACGCACT | 46 | 0150-0169 | 5′ UTR |
| 31719 | GCCAGGGCACTGGGCGCTCG | 47 | 0158-0177 | 5′ UTR |
| 31397 | TCTCCGGGCCAGGGCACTGG | 48 | 0165-0184 | 5′ UTR |
| 31720 | TCATTCCACTCTCCGGGCCA | 49 | 0174-0193 | 5′ UTR |
| 31556 | GGAAGCACGACGCCCTGGGC | 50 | 0202-0221 | 5′ UTR |
| 31721 | TACTGCGGAAGCACGACGCC | 51 | 0208-0227 | 5′ UTR |
| 31398 | GGGACTGACTACTGCGGAAG | 52 | 0217-0236 | 5′ UTR |
| 31722 | TCAAGACTCCCCAGTTTCCT | 53 | 0242-0261 | 5′ UTR |
| 31557 | CCTGCTCCTCACCATCCGGG | 54 | 0289-0308 | 5′ UTR |
| 31399 | TTTGCCTGCTCCTCACCATC | 55 | 0293-0312 | AUG |
| 31400 | ATTTGCCTGCTCCTCACCAT | 56 | 0294-0313 | AUG |
| 31401 | CATTTGCCTGCTCCTCACCA | 9 | 0295-0314 | AUG |
| 31402 | ACATTTGCCTGCTCCTCACC | 57 | 0296-0315 | AUG |
| 31403 | CACATTTGCCTGCTCCTCAC | 58 | 0297-0316 | AUG |
| 31404 | GCACATTTGCCTGCTCCTCA | 59 | 0298-0317 | AUG |
| 31405 | TGCACATTTGCCTGCTCCTC | 60 | 0299-0318 | AUG |
| 31406 | TTGCACATTTGCCTGCTCCT | 61 | 0300-0319 | AUG |
| 31407 | ATTGCACATTTGCCTGCTCC | 62 | 0301-0320 | AUG |
| 31408 | TATTGCACATTTGCCTGCTC | 63 | 0302-0321 | AUG |
| 31409 | GTATTGCACATTTGCCTGCT | 10 | 0303-0322 | AUG |
| 31410 | GGTATTGCACATTTGCCTGC | 64 | 0304-0323 | AUG |
| 31411 | TGGTATTGCACATTTGCCTG | 65 | 0305-0324 | AUG |
| 31412 | TTGGTATTGCACATTTGCCT | 66 | 0306-0325 | AUG |
| 31413 | GTTGGTATTGCACATTTGCC | 67 | 0307-0326 | AUG |
| 31414 | TGTTGGTATTGCACATTTGC | 68 | 0308-0327 | AUG |
| 31415 | ATGTTGGTATTGCACATTTG | 69 | 0309-0328 | AUG |
| 31416 | CATGTTGGTATTGCACATTT | 70 | 0310-0329 | AUG |
| 31417 | ACATGTTGGTATTGCACATT | 71 | 0311-0330 | AUG |
| 31418 | GACATGTTGGTATTGCACAT | 72 | 0312-0331 | AUG |
| 31419 | AGACATGTTGGTATTGCACA | 73 | 0313-0332 | AUG |
| 31420 | CAGACATGTTGGTATTGCAC | 74 | 0314-0333 | AUG |
| 31558 | CAGTAGGTACAGACATGTTG | 75 | 0323-0342 | coding |
| 31723 | TACAGCACCATCAGTAGGTA | 76 | 0334-0353 | coding |
| 31421 | GGAATCTGTGAGGTGGTTAC | 77 | 0351-0370 | coding |
| 31559 | TTCCGAAGCTGGAATCTGTG | 78 | 0361-0380 | coding |
| 31724 | AGGGTCTCTTGTTCCGAAGC | 79 | 0372-0391 | coding |
| 31422 | GCTTTGGTCTAACCAGGGTC | 80 | 0386-0405 | coding |
| 31560 | GCAATGGCTTTGGTCTAACC | 81 | 0392-0411 | coding |
| 31725 | TAACTTCAAAAGCAATGGCT | 82 | 0403-0422 | coding |
| 31423 | GTGCACCAACAGACTTTAAT | 83 | 0422-0441 | coding |
| 31561 | ACCTCTTTCATAGTATAAGT | 84 | 0450-0469 | coding |
| 31726 | ATAATATACTGGCCAAGATA | 85 | 0477-0496 | coding |
| 31424 | TAATCGTTTAGTCATAATAT | 86 | 0490-0509 | coding |
| 31727 | ATCATATAATCGTTTAGTCA | 87 | 0496-0515 | coding |
| 31562 | GCTTCTCATCATATAATCGT | 88 | 0503-0522 | coding |
| 31728 | CAATATGTTGTTGCTTCTCA | 89 | 0515-0534 | coding |
| 31425 | GAACAATATACAATATGTTG | 90 | 0525-0544 | coding |
| 31729 | TCATTTGAACAATATACAAT | 91 | 0531-0550 | coding |
| 31563 | TAGAAGATCATTTGAACAAT | 92 | 0538-0557 | coding |
| 31730 | AACAAATCTCCTAGAAGATC | 93 | 0549-0568 | coding |
| 31426 | TGGCACGCCAAACAAATCTC | 94 | 0559-0578 | coding |
| 31731 | AGAAGCTTGGCACGCCAAAC | 95 | 0566-0585 | coding |
| 31564 | CTTTCACAGAGAAGCTTGGC | 96 | 0575-0594 | coding |
| 31732 | TTTTCCTGTGCTCTTTCACA | 97 | 0587-0606 | coding |
| 31427 | TATATATTTTCCTGTGCTCT | 98 | 0593-0612 | coding |
| 31733 | ATCATGGTATATATTTTCCT | 99 | 0600-0619 | coding |
| 31565 | TTCCTGTAGATCATGGTATA | 100 | 0609-0628 | coding |
| 31734 | TACTACCAAGTTCCTGTAGA | 101 | 0619-0638 | coding |
| 31428 | TTCCTGCTGATTGACTACTA | 102 | 0634-0653 | coding |
| 31566 | TGAGTCCGATGATTCCTGCT | 103 | 0646-0665 | coding |
| 31735 | CAGATGTACCTGAGTCCGAT | 104 | 0656-0675 | coding |
| 31429 | CTGTTCTCACTCACAGATGT | 105 | 0669-0688 | coding |
| 31567 | TTCAAGGTGACACCTGTTCT | 106 | 0682-0701 | coding |
| 31736 | ACTCCCACCTTCAAGGTGAC | 107 | 0691-0710 | coding |
| 31430 | GGTCCTTTTGATCACTCCCA | 108 | 0704-0723 | coding |
| 31568 | AAGCTCTTGTACAAGGTCCT | 109 | 0718-0737 | coding |
| 31737 | CTCTTCCTGAAGCTCTTGTA | 110 | 0727-0746 | coding |
| 31431 | AAGATGAAGGTTTCTCTTCC | 111 | 0740-0759 | coding |
| 31569 | AAACCAAATGTGAAGATGAA | 112 | 0752-0771 | coding |
| 31738 | ATGGTCTAGAAACCAAATGT | 113 | 0761-0780 | coding |
| 31432 | CTAGATGAGGTAGATGGTCT | 114 | 0774-0793 | coding |
| 31570 | AATTGCTCTCCTTCTAGATG | 115 | 0787-0806 | coding |
| 31739 | TCTGTCTCACTAATTGCTCT | 116 | 0798-0817 | coding |
| 31433 | TCTGAATTTTCTTCTGTCTC | 117 | 0810-0829 | coding |
| 31571 | CACCAGATAATTCATCTGAA | 118 | 0824-0843 | coding |
| 31740 | TTTGTCGTTCACCAGATAAT | 119 | 0833-0852 | coding |
| 31434 | GTGGCGTTTTCTTTGTCGTT | 120 | 0844-0863 | coding |
| 31572 | TACTATCAGATTTGTGGCGT | 121 | 0857-0876 | coding |
| 31741 | GAAAGGGAAATACTATCAGA | 122 | 0867-0886 | coding |
| 31435 | GCTTTCATCAAAGGAAAGGG | 123 | 0880-0899 | coding |
| 31573 | TACACACAGAGCCAGGCTTT | 124 | 0895-0914 | coding |
| 31742 | CTCCCTTATTACACACAGAG | 125 | 0904-0923 | coding |
| 31436 | TCACAACATATCTCCCTTAT | 126 | 0915-0934 | coding |
| 31574 | CTACTGCTTCTTTCACAACA | 127 | 0927-0946 | coding |
| 31743 | GATTCACTGCTACTGCTTCT | 128 | 0936-0955 | coding |
| 31437 | TGGCGTCCCTGTAGATTCAC | 129 | 0949-0968 | coding |
| 31575 | AAGATCCGGATTCGATGGCG | 130 | 0964-0983 | coding |
| 31744 | CAGCATCAAGATCCGGATTC | 131 | 0971-0990 | coding |
| 31438 | GTTCACTTACACCAGCATCA | 132 | 0983-1002 | coding |
| 31576 | CAATCACCTGAATGTTCACT | 133 | 0996-1015 | coding |
| 31745 | CTGATCCAACCAATCACCTG | 134 | 1006-1025 | coding |
| 31439 | GAAACTGAATCCTGATCCAA | 135 | 1017-1036 | coding |
| 31746 | TGATCTGAAACTGAATCCTG | 136 | 1023-1042 | coding |
| 31577 | CTACACTAAACTGATCTGAA | 137 | 1034-1053 | coding |
| 31747 | CAACTTCAAATTCTACACTA | 138 | 1046-1065 | coding |
| 31440 | AGATTCAACTTCAAATTCTA | 139 | 1051-1070 | coding |
| 31748 | GAGTCGAGAGATTCAACTTC | 140 | 1059-1078 | coding |
| 31578 | TAATCTTCTGAGTCGACAGA | 141 | 1068-1087 | coding |
| 31749 | CTAAGGCTATAATCTTCTGA | 142 | 1077-1096 | coding |
| 31441 | TTCTTCACTAAGGCTATAAT | 143 | 1084-1103 | coding |
| 31750 | TCTTGTCCTTCTTCACTAAG | 144 | 1092-1111 | coding |
| 31579 | CTGAGAGTTCTTGTCCTTCT | 145 | 1100-1119 | coding |
| 31751 | TTCATCTGAGAGTTCTTGTC | 146 | 1105-1124 | coding |
| 31442 | CCTCATCATCTTCATCTGAG | 147 | 1115-1134 | coding |
| 31752 | CTTGATATACCTCATCATCT | 148 | 1124-1143 | coding |
| 31753 | ATACACAGTAACTTGATATA | 149 | 1135-1154 | coding |
| 31443 | CTCTCCCCTGCCTGATACAC | 150 | 1149-1168 | coding |
| 31580 | GAATCTGTATCACTCTCCCC | 151 | 1161-1180 | coding |
| 31754 | TCTTCAAATGAATCTGTATC | 152 | 1170-1189 | coding |
| 31444 | AAATTTCAGGATCTTCTTCA | 153 | 1184-1203 | coding |
| 31581 | AGTCAGCTAAGGAAATTTCA | 154 | 1196-1215 | coding |
| 31755 | GCATTTCCAATAGTCAGCTA | 155 | 1207-1226 | coding |
| 31445 | CATTGCATGAAGTGCATTTC | 156 | 1220-1239 | coding |
| 31756 | TCATTTCATTGCATGAAGTG | 157 | 1226-1245 | coding |
| 31582 | CATCTGTTGCAATGTGATGG | 158 | 1257-1276 | coding |
| 31757 | GAAGGGCCCAACATCTGTTG | 159 | 1268-1287 | coding |
| 31446 | TTCTCACGAAGGGCCCAACA | 160 | 1275-1294 | coding |
| 31758 | GAAGCCAATTCTCACGAAGG | 161 | 1283-1302 | coding |
| 31583 | TATCTTCAGGAAGCCAATTC | 162 | 1292-1311 | coding |
| 31759 | CTTTCCCTTTATCTTCAGGA | 163 | 1301-1320 | coding |
| 31447 | TCCCCTTTATCTTTCCCTTT | 164 | 1311-1330 | coding |
| 31584 | CTTTCTCAGAGATTTCCCCT | 165 | 1325-1344 | coding |
| 31760 | CAGTTTGGCTTTCTCAGAGA | 166 | 1333-1352 | coding |
| 31448 | GTGTTGAGTTTTCCAGTTTG | 167 | 1346-1365 | coding |
| 31585 | CCTCTTCAGCTTGTGTTGAG | 168 | 1358-1377 | coding |
| 31761 | ACATCAAAGCCCTCTTCAGC | 169 | 1368-1787 | coding |
| 31449 | GAATCATTCACTATAGTTTT | 170 | 1401-1420 | coding |
| 31586 | ATGACTCTCTGGAATCATTC | 171 | 1412-1431 | coding |
| 31762 | CCTCAACACATGACTCTCTG | 172 | 1421-1440 | coding |
| 31450 | TTATCATCATTTTCCTCAAC | 173 | 1434-1453 | coding |
| 31763 | TAATTTTATCATCATTTTCC | 174 | 1439-1458 | coding |
| 31587 | GAAGCTTGTGTAATTTTATC | 175 | 1449-1468 | coding |
| 31764 | TGATTGTGAAGCTTGTGTAA | 176 | 1456-1475 | coding |
| 31451 | CACTTTCTTGTGATTGTGAA | 177 | 1466-1485 | coding |
| 31588 | GCTGAGAATAGTCTTCACTT | 178 | 1481-1500 | coding |
| 31765 | AGTTGATGGCTGAGAATAGT | 179 | 1489-1508 | coding |
| 31452 | TGCTACTAGAAGTTGATGGC | 180 | 1499-1518 | coding |
| 31766 | TAAATAATGCTACTAGAAGT | 181 | 1506-1525 | coding |
| 31589 | CTTGGCTGCTATAAATAATG | 182 | 1517-1536 | coding |
| 31590 | ATCTTCTTGGCTGCTATAAA | 183 | 1522-1541 | coding |
| 31453 | AACTCTTTCACATCTTCTTG | 184 | 1533-1552 | coding |
| 31767 | CCCTTTCAAACTCTTTCACA | 185 | 1541-1560 | coding |
| 31591 | GGGTTTCTTCCCTTTCAAAC | 186 | 1550-1569 | coding |
| 31768 | TCTTTGTCTTGGGTTTCTTC | 187 | 1560-1579 | coding |
| 31454 | CTCTCTTCTTTGTCTTGGGT | 188 | 1566-1585 | coding |
| 31592 | AACTAGATTCCACACTCTCT | 189 | 1580-1599 | coding |
| 31769 | CAAGATTCAATGGCATTAAG | 190 | 1605-1624 | coding |
| 31455 | TGACAAATCACACAAGGTTC | 191 | 1617-1636 | coding |
| 31593 | TCGACCTTGACAAATCACAC | 192 | 1624-1643 | coding |
| 31594 | ATGGACAATGCAACCATTTT | 193 | 1648-1667 | coding |
| 31770 | TGTTTTGCCATGGACAATGC | 194 | 1657-1676 | coding |
| 31456 | TAAGATGTCCTGTTTTGCCA | 195 | 1667-1686 | coding |
| 31595 | GCAGGCCATAAGATGTCCTG | 196 | 1675-1694 | coding |
| 31596 | ACATGTAAAGCAGGCCATAA | 197 | 1684-1703 | coding |
| 31771 | CTTTGCACATGTAAAGCAGG | 198 | 1690-1709 | coding |
| 31457 | TTTCTTTAGCTTCTTTGCAC | 199 | 1702-1721 | coding |
| 31597 | TTATTCCTTTTCTTTAGCTT | 200 | 1710-1729 | coding |
| 31598 | TGGGCAGGGCTTATTCCTTT | 201 | 1720-1739 | coding |
| 31772 | ACATACTGGGCAGGGCTTAT | 202 | 1726-1745 | coding |
| 31458 | TTGGTTGTCTACATACTGGG | 203 | 1736-1755 | coding |
| 31599 | TCATTTGAATTGGTTGTCTA | 204 | 1745-1764 | coding |
| 31600 | AAGTTAGCACAATCATTTGA | 205 | 1757-1776 | coding |
| 31601 | TCTCTTATAGACAGGTCAAC | 206 | 1787-1806 | STOP |
| 31459 | AAATATATAATTCTCTTATA | 207 | 1798-1817 | 3′ UTR |
| 31602 | AGTTAGAAATATATAATTCT | 208 | 1804-1823 | 3′ UTR |
| 31773 | ATATAGTTAGAAATATATAA | 209 | 1808-1827 | 3′ UTR |
| 31603 | CTAGGGTTATATAGTTAGAA | 210 | 1816-1835 | 3′ UTR |
| 31774 | TAAATTCCTAGGGTTATATA | 211 | 1823-1842 | 3′ UTR |
| 31460 | CAGGTTGTCTAAATTCCTAG | 212 | 1832-1851 | 3′ UTR |
| 31604 | ATAAATTTCAGGTTGTCTAA | 213 | 1840-1859 | 3′ UTR |
| 31605 | ATATATGTGAATAAATTTCA | 214 | 1850-1869 | 3′ UTR |
| 31606 | CTTTGATATATGTGAATAAA | 215 | 1855-1874 | 3′ UTR |
| 31461 | CATTTTCTCACTTTGATATA | 216 | 1865-1884 | 3′ UTR |
| 31607 | ATTGAGGCATTTTCTCACTT | 217 | 1872-1891 | 3′ UTR |
| 31608 | AATCTATGTGAATTGAGGCA | 218 | 1883-1902 | 3′ UTR |
| 31609 | AGAAGAAATCTATGTGAATT | 219 | 1889-1908 | 3′ UTR |
| 31462 | ATACTAAAGAGAAGAAATCT | 220 | 1898-1917 | 3′ UTR |
| 31610 | GTCAATTATACTAAAGAGAA | 221 | 1905-1924 | 3′ UTR |
| 31775 | TAGGTCAATTATACTAAAGA | 222 | 1908-1927 | 3′ UTR |
| 31611 | CAAAGTAGGTCAATTATACT | 223 | 1913-1932 | 3′ UTR |
| 31776 | CCACTACCAAAGTAGGTCAA | 224 | 1920-1939 | 3′ UTR |
| 31463 | AGTATTCACTATTCCACTAC | 225 | 1933-1952 | 3′ UTR |
| 31612 | TATAGTAAGTATTCACTATT | 226 | 1940-1959 | 3′ UTR |
| 31613 | AGTCAAATTATAGTAAGTAT | 227 | 1948-1967 | 3′ UTR |
| 31777 | CATATTCAAGTCAAATTATA | 228 | 1956-1975 | 3′ UTR |
| 31464 | AAAGGATGAGCTACATATTC | 229 | 1969-1988 | 3′ UTR |
| 31778 | GTGTAAAGGATGAGCTACAT | 230 | 1973-1992 | 3′ UTR |
| 31614 | TAGGAGTTGGTGTAAAGGAT | 231 | 1982-2001 | 3′ UTR |
| 31779 | TTTAAAATTAGGAGTTGGTG | 232 | 1990-2009 | 3′ UTR |
| 31615 | GAAATTATTTAAAATTAGGA | 233 | 1997-2016 | 3′ UTR |
| 31465 | CAGAGTAGAAATTATTTAAA | 234 | 2004-2023 | 3′ UTR |
| 31616 | CTCATTTAAGACAGAGTAGA | 235 | 2015-2034 | 3′ UTR |
| 31780 | TACTTCTCATTTAAGACAGA | 236 | 2020-2039 | 3′ UTR |
| 31617 | CATATACATATTTAAGAAAA | 237 | 2051-2070 | 3′ UTR |
| 31466 | TTAAATGTCATATACATATT | 238 | 2059-2078 | 3′ UTR |
| 31618 | TAATAAGTTACATTTAAATG | 239 | 2072-2091 | 3′ UTR |
| 31619 | GTAACAGAGCAAGACTCGGT | 240 | 2103-2122 | 3′ UTR |
| 31467 | CAGCCTGGGTAACAGAGCAA | 241 | 2111-2130 | 3′ UTR |
| 31781 | CACTCCAGCCTGGGTAACAG | 242 | 2116-2135 | 3′ UTR |
| 31620 | CCCACTGCACTCCAGCCTGG | 243 | 2123-2142 | 3′ UTR |
| 31782 | GCCAAGATCACCCACTGCAC | 244 | 2133-2152 | 3′ UTR |
| 31621 | GCAGTGAGCCAAGATCACCC | 245 | 2140-2159 | 3′ UTR |
| 31468 | GAGCTTGCAGTGAGCCAAGA | 246 | 2146-2165 | 3′ UTR |
| 31783 | GAGGGCAGAGCTTGCAGTGA | 247 | 2153-2172 | 3′ UTR |
| 31622 | CAGGAGAATGGTGCGAACCC | 248 | 2176-2195 | 3′ UTR |
| 31623 | AGGCTGAGGCAGGAGAATGG | 249 | 2185-2204 | 3′ UTR |
| 31784 | ATTGGGAGGCTGAGGCAGGA | 250 | 2191-2210 | 3′ UTR |
| 31469 | CAAGCTAATTGGGAGGCTGA | 251 | 2198-2217 | 3′ UTR |
| 31624 | AGGCCAAGCTAATTGGGAGG | 252 | 2202-2221 | 3′ UTR |
| 31785 | ATGACTGTAGGCCAAGCTAA | 253 | 2210-2229 | 3′ UTR |
| 31625 | CAGATGACTGTAGGCCAAGC | 254 | 2213-2232 | 3′ UTR |
| 31786 | GGTGGCAGATGACTGTAGGC | 255 | 2218-2237 | 3′ UTR |
| 31626 | AGGTGTGGTGGCAGATGACT | 21 | 2224-2243 | 3′ UTR |
| 31470 | AATTAGCCAGGTGTGGTGGC | 256 | 2232-2251 | 3′ UTR |
| 31627 | GTCTCTACTAAAAGTACAAA | 257 | 2253-2272 | 3′ UTR |
| 31628 | CGGTGAAACCCTGTCTCTAC | 258 | 2265-2284 | 3′ UTR |
| 31787 | TGGCTAACACGGTGAAACCC | 259 | 2274-2293 | 3′ UTR |
| 31471 | AGACCATCCTGGCTAACACG | 260 | 2283-2302 | 3′ UTR |
| 31788 | GAGATCGAGACCATCCTGGC | 261 | 2290-2309 | 3′ UTR |
| 31629 | GAGGTCAGGAGATCGAGACC | 262 | 2298-2317 | 3′ UTR |
| 31789 | GCGGATCACGAGGTCAGGAG | 263 | 2307-2326 | 3′ UTR |
| 31472 | AGGCCGAGGTGGGCGGATCA | 264 | 2319-2338 | 3′ UTR |
| 31790 | TTTGGGAGGCCGAGGTGGGC | 265 | 2325-2344 | 3′ UTR |
| 31630 | TCCCAGCACTTTGGGAGGCC | 266 | 2334-2353 | 3′ UTR |
| 31791 | CCTGTAATCCCAGCACTTTG | 267 | 2341-2360 | 3′ UTR |
| 31631 | GTGGCTCATGCCTGTAATCC | 268 | 2351-2370 | 3′ UTR |

¹All deoxy cytosines and 2′-MOE cytosine residues are 5-methyl-cytosines; all linkages are phosphorothioate linkages.
²Co-ordinates from Genbank Accession No. Z12020, locus name “HSP53ASSG”, SEQ ID NO:1.

Oligonucleotide activity was assayed by quantitation of mdm2 mRNA levels by real-time PCR (RT-PCR) using the ABI PRISM™ 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR, in which amplification products are quantitated after the PCR is completed, products in RT-PCR are quantitated as they accumulate. This is accomplished by including in the PCR reaction an oligonucleotide probe that anneals specifically between the forward and reverse PCR primers, and contains two fluorescent dyes. The primers and probes used were:

(SEQ ID NO. 269)

| | |
|---|---|
| Forward: | 5′-GGCAAATGTGCAATACCAACA-3′ |

(SEQ ID NO. 270)

| | |
|---|---|
| Reverse: | 5′-TGCACCAACAGACTTTAATAACTTCA-3′ |

(SEQ ID NO. 271).

| | |
|---|---|
| Probe: | 5′-FAM-CCACCTCACAGATTCCAGCTTCGGA-TAMRA-3′ |

A reporter dye (e.g., JOE or FAM, PE-Applied Biosystems, Foster City, Calif.) was attached to the 5′ end of the probe and a quencher dye (e.g., TAMRA, PE-Applied Biosystems, Foster City, Calif.) was attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular (six-second) intervals by laser optics built into the ABI PRISM™ 7700 Sequence Detection System. In each assay, a series of parallel reactions containing serial dilutions of mRNA from untreated control samples generates a standard curve that is used to quantitate the percent inhibition after antisense oligonucleotide treatment of test samples.

RT-PCR reagents were obtained from PE-Applied Biosystems, Foster City, Calif. RT-PCR reactions were carried out by adding 25 μl PCR cocktail (1× TAQMAN™ buffer A, 5.5 mM MgCl₂, 300 μM each of DATP, dCTP and dGTP, 600 μM of dUTP, 100 nM each of forward primer, reverse primer, and probe, 20 U RNAse inhibitor, 1.25 units AMPLITAQ GOLD™, and 12.5 U MuLV reverse transcriptase) to 96 well plates containing 25 μl poly(A) mRNA solution. The RT reaction was carried out by incubation for 30 minutes at 48° C. Following a 10 minute incubation at 95° C. to activate the AMPLITAQ GOLD™, 40 cycles of a two-step PCR protocol were carried out: 95° C. for 15 seconds (denaturation) followed by 60° C. for 1.5 minutes (annealing/extension).

Results are shown in Table 12. Oligonucleotides 31394 (SEQ ID NO: 36), 31398 (SEQ ID NO: 52), 31400 (SEQ ID NO: 56), 31402 (SEQ ID NO: 57), 31405 (SEQ ID NO: 60), 31406 (SEQ ID NO: 61), 31415 (SEQ ID NO: 69), 31416 (SEQ ID NO: 70), 31418 (SEQ ID NO: 72), 31434 (SEQ ID NO: 60), 31436 (SEQ ID NO: 126), 31446 (SEQ ID NO: 160), 31451 (SEQ ID NO: 177), 31452 (SEQ ID NO: 180), 31456 (SEQ ID NO: 195), 31461 (SEQ ID NO: 216), 31468 (SEQ ID NO: 246), 31469 (SEQ ID NO: 251), 31471 (SEQ ID NO: 260), and 31472 (SEQ ID NO: 264) gave at least approximately 50% reduction of mdm2 mRNA levels.

TABLE 12
Activities of Phosphorothioate Oligodeoxynucleotides
Targeted to Human mdm2

| | SEQ | GENE | | |
|---|---|---|---|---|
| | ID | TARGET | % mRNA | % mRNA |
| ISIS No: | NO: | REGION | EXPRESSION | INHIBITION |
| LIPOFECTIN ™ | — | — | 100% | 0% |
| only | | | | |
| 31393 | 3 | 5′ UTR | 59% | 41% |
| 31394 | 36 | 5′ UTR | 27% | 73% |
| 31395 | 40 | 5′ UTR | 96% | 4% |
| 31396 | 44 | 5′ UTR | 99% | 1% |
| 31397 | 48 | 5′ UTR | 76% | 24% |
| 31398 | 52 | 5′ UTR | 51% | 49% |
| 31399 | 55 | AUG | 138% | — |
| 31400 | 56 | AUG | 22% | 78% |
| 31401 | 9 | AUG | 69% | 31% |
| 31402 | 57 | AUG | 47% | 53% |
| 31403 | 58 | AUG | 77% | 23% |
| 31404 | 59 | AUG | 60% | 40% |
| 31405 | 60 | AUG | 35% | 65% |
| 31406 | 61 | AUG | 45% | 55% |
| 31407 | 62 | AUG | 65% | 35% |
| 31408 | 63 | AUG | 71% | 29% |
| 31409 | 10 | AUG | 849% | — |
| 31410 | 64 | AUG | 79% | 21% |
| 31411 | 65 | AUG | 67% | 33% |
| 31412 | 66 | AUG | 99% | 1% |
| 31413 | 67 | AUG | 68% | 32% |
| 31414 | 68 | AUG | 64% | 36% |
| 31415 | 69 | AUG | 48% | 52% |
| 31416 | 70 | AUG | 36% | 64% |
| 31417 | 71 | AUG | 77% | 23% |
| 31418 | 72 | AUG | 53% | 47% |
| 31419 | 73 | AUG | 122% | — |
| 31420 | 74 | AUG | 57% | 43% |
| 31421 | 77 | coding | 111% | — |
| 31422 | 80 | coding | 85% | 15% |
| 31423 | 83 | coding | 126% | — |
| 31424 | 86 | coding | 70% | 30% |
| 31425 | 90 | coding | 95% | 5% |
| 31426 | 94 | coding | 69% | 31% |
| 31427 | 98 | coding | 9465% | — |
| 31428 | 102 | coding | 81% | 19% |
| 31429 | 105 | coding | 138% | — |
| 31430 | 108 | coding | 114% | — |
| 31431 | 111 | coding | 77% | 23% |
| 31432 | 114 | coding | 676% | — |
| 31433 | 117 | coding | 145% | — |
| 31434 | 120 | coding | 40% | 60% |
| 31435 | 123 | coding | 193% | — |
| 31436 | 126 | coding | 49% | 51% |
| 31437 | 129 | coding | 146% | — |
| 31438 | 132 | coding | 76% | 24% |
| 31439 | 135 | coding | 104% | — |
| 31440 | 139 | coding | 95% | 5% |
| 31441 | 143 | coding | 324% | — |
| 31442 | 147 | coding | 1840% | — |
| 31443 | 150 | coding | 369% | — |
| 31444 | 153 | coding | 193% | — |
| 31445 | 156 | coding | 106% | — |
| 31446 | 160 | coding | 29% | 71% |
| 31447 | 164 | coding | 82% | 18% |
| 31448 | 167 | coding | 117% | — |
| 31449 | 170 | coding | 1769% | — |
| 31450 | 173 | coding | 84% | 16% |
| 31451 | 177 | coding | 49% | 51% |
| 31452 | 180 | coding | 33% | 67% |
| 31453 | 184 | coding | 59% | 41% |
| 31454 | 188 | coding | 171% | — |
| 31455 | 191 | coding | 61% | 39% |
| 31456 | 195 | coding | 42% | 58% |
| 31457 | 199 | coding | 70% | 30% |
| 31458 | 203 | coding | 60% | 40% |
| 31459 | 207 | 3′ UTR | 149% | — |
| 31460 | 212 | 3′ UTR | 71% | 29% |
| 31461 | 216 | 3′ UTR | 52% | 48% |
| 31462 | 220 | 3′ UTR | 1113% | — |
| 31463 | 225 | 3′ UTR | 78% | 22% |
| 31464 | 229 | 3′ UTR | 112% | — |
| 31465 | 234 | 3′ UTR | 66% | 34% |
| 31466 | 238 | 3′ UTR | 212% | — |
| 31467 | 241 | 3′ UTR | 77% | 23% |
| 31468 | 246 | 3′ UTR | 17% | 83% |
| 31469 | 251 | 3′ UTR | 36% | 64% |
| 31470 | 256 | 3′ UTR | 60% | 40% |
| 31471 | 260 | 3′ UTR | 43% | 57% |
| 31472 | 264 | 3′ UTR | 35% | 65% |

### Example 10

Effect of mdm2 antisense oligonucleotides on the growth of human A549 lung tumor cells in nude mice

200 μl of A549 cells (5×106 cells) are implanted subcutaneously in the inner thigh of nude mice. mdm2 antisense oligonucleotides are administered twice weekly for four weeks, beginning one week following tumor cell inoculation. Oligonucleotides are formulated with cationic lipids (LIPOFECTIN™) and given subcutaneously in the vicinity of the tumor. Oligonucleotide dosage was 5 mg/kg with 60 mg/kg cationic lipid. Tumor size is recorded weekly.

Activity of the oligonucleotides is measured by reduction in tumor size compared to controls.

### Example 11

U-87 human glioblastoma cell culture and subcutaneous xenografts into nude mice

The U-87 human glioblastoma cell line is obtained from the ATCC (Manassas, Va.) and maintained in Iscove's DMEM medium supplemented with heat-inactivated 10% fetal calf serum (Yazaki, T., et al., Mol. Pharmacol., 1996, 50, 236-242). Nude mice are injected subcutaneously with 2×10⁷ cells. Mice are injected intraperitoneally with oligonucleotide at dosages of either 2 mg/kg or 20 mg/kg for 21 consecutive days beginning 7 days after xenografts were implanted. Tumor volumes are measured on days 14, 21, 24, 31 and 35. Activity is measure by a reduced tumor volume compared to saline or sense oligonucleotide controls.

### Example 12

Intracerebral U-87 glioblastoma xenografts into nude mice

U-87 cells are implanted in the brains of nude mice (Yazaki, T., et al., Mol. Pharmacol., 1996, 50, 236-242). Mice are treated via continuous intraperitoneal administration of antisense oligonucleotide (20 mg/kg), control sense oligonucleotide (20 mg/kg) or saline beginning on day 7 after xenograft implantation. Activity of the oligonucleotide is measured by an increased survival time compared to controls.

### Example 13

Analysis of oligonucleotide inhibition of mdm2 expression in T-24 cells

The effect of antisense compounds on target nucleic acid expression can be tested in any of a variety of cell types provided that the target nucleic acid is present at measurable levels. This can be routinely determined using, for example, PCR or Northern blot analysis. T-24 cells are provided for illustrative purposes, but other cell types can be routinely used, provided that the target is expressed in the cell type chosen. This can be readily determined by methods routine in the art, for example Northern blot analysis, Ribonuclease protection assays, or RT-PCR.

T-24 cells:

The human transitional cell bladder carcinoma cell line T-24 was obtained from the American Type Culture Collection (ATCC) (Manassas, Va.). T-24 cells were routinely cultured in complete McCoy's 5A basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells were routinely passaged by trypsinization and dilution when they reached 90% confluence. Cells were seeded into 96-well plates (Falcon-Primaria #3872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

Treatment with antisense compounds:

When cells reached 80% confluency, they were treated with oligonucleotide. For cells grown in 96-well plates, wells were washed once with 200 μL OPTI-MEM™-1 reduced-serum medium (Gibco BRL) and then treated with 130 μL of OPTI-MEM™-1 containing 3.75 g/mL LIPOFECTIN™ (Gibco BRL) and the desired concentration of oligonucleotide. After 4-7 hours of treatment, the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is ISIS 13920, TCCGTCATCGCTCCTCAGGG, SEQ ID NO: 272, a 2′-O-methoxyethyl gapmer (2′-O-methoxyethyls shown in bold) with a phosphorothioate backbone which is targeted to human H-ras.

The concentration of positive control oligonucleotide that results in 80% inhibition of c-Ha-ras (for ISIS 13920) mRNA is then utilized as the screening concentration for new oligonucleotides in subsequent experiments for that cell line. If 80% inhibition is not achieved, the lowest concentration of positive control oligonucleotide that results in 60% inhibition of H-ras or c-raf mRNA is then utilized as the oligonucleotide screening concentration in subsequent experiments for that cell line. If 60% inhibition is not achieved, that particular cell line is deemed as unsuitable for oligonucleotide transfection experiments.

Analysis of oligonucleotide inhibition of mdm2 expression:

Antisense modulation of mdm2 expression can be assayed in a variety of ways known in the art. For example, mdm2 mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions.

Protein levels of mdm2 can be quantitated in a variety of ways well known in the art, such as immunoprecipitation, Western blot analysis (immunoblotting), ELISA or fluorescence-activated cell sorting (FACS). Antibodies directed to mdm2 can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

Poly(A)+ mRNA isolation:

Poly(A)+ mRNA is isolated according to Miura et al., Clin. Chem., 1996, 42, 1758-1764. Other methods for poly(A)+ mRNA isolation are taught in, for example, Ausubel, F. M. et al., Current Protocols in Molecular Biology, Volume 1, pp. 4.5.1-4.5.3, John Wiley & Sons, Inc., 1993. Briefly, for cells grown on 96-well plates, growth medium was removed from the cells and each well was washed with 200 μL cold PBS. 60 μL lysis buffer (10 mM Tris-HC1, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C. was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

Total RNA Isolation:

Total RNA is isolated using an RNEASY 96™ kit and buffers purchased from Qiagen Inc. (Valencia Calif.) following the manufacturer's recommended procedures. Briefly, for cells grown on 96-well plates, growth medium was removed from the cells and each well was washed with 200 μL cold PBS. 100 μL Buffer RLT was added to each well and the plate vigorously agitated for 20 seconds. 100 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 15 seconds. 1 mL of Buffer RW1 was added to each well of the RNEASY 96™ plate and the vacuum again applied for 15 seconds. 1 mL of Buffer RPE was then added to each well of the RNEASY 96™ plate and the vacuum applied for a period of 15 seconds. The Buffer RPE wash was then repeated and the vacuum was applied for an additional 10 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 60 μL water into each well, incubating 1 minute, and then applying the vacuum for 30 seconds. The elution step was repeated with an additional 60 μL water.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 14

Real-time Quantitative PCR Analysis of Human mdm2 mRNA Levels

Quantitation of mdm2 mRNA levels was determined by real-time quantitative PCR using the ABI PRISM™ 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE, FAM, or VIC, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ 7700 Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from PE-Applied Biosystems, Foster City, Calif. RT-PCR reactions were carried out by adding 25 μL PCR cocktail (1× TAQMANT buffer A, 5.5 mM MgCl₂, 300 μM each of DATP, dCTP and dGTP, 600 μM of dUTP, 100 nM each of forward primer, reverse primer, and probe, 20 Units RNAse inhibitor, 1.25 Units AMPLITAQ GOLD™, and 12.5 Units MuLV reverse transcriptase) to 96 well plates containing 25 μL total RNA solution. The RT reaction was carried out by incubation for 30 minutes at 48° C. Following a 10 minute incubation at 95° C. to activate the AMPLITAQ GOLD™, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent from Molecular Probes. Methods of RNA quantification by RiboGreen™ are taught in Jones, L. J., et al, Analytical Biochemistry, 1998, 265, 368-374.

In this assay, 175 μL of RiboGreen™ working reagent (RiboGreen™ reagent diluted 1:2865 in 10 mM Tris-HCl, 1 mM EDTA, pH 7.5) is pipetted into a 96-well plate containing 25uL purified, cellular RNA. The plate is read in a CytoFluor 4000 (PE Applied Biosystems) with excitation at 480 nm and emission at 520 nm.

Probes and primers to human mdm2 were designed to hybridize to a human mdm2 sequence, using published sequence information (GenBank accession number Z12020, incorporated herein as SEQ ID NO:1). For human mdm2 the PCR primers were:

| | |
|---|---|
| forward primer: | |
| GGCAAATGTGCAATACCAACA | (SEQ ID NO: 269) |
| reverse primer: | |
| TGCACCAACAGACTTTAATAACTTCA | (SEQ ID NO: 270) |

and the PCR probe was: FAM-CCACCTCACAGATTCCAGCTTCGGA-TAMRA (SEQ ID NO: 271) where FAM (PE-Applied Biosystems, Foster City, Calif.) is the fluorescent reporter dye) and TAMRA (PE-Applied Biosystems, Foster City, Calif.) is the quencher dye. For human GAPDH the PCR primers were:

| | | |
|---|---|---|
| forward primer: | CAACGGATTTGGTCGTATTGG | (SEQ ID NO: 273) |
| reverse primer: | GGCAACAATATCCACTTTACCAGAGT | (SEQ ID NO: 274) |

and the PCR probe was: 5′ JOE-CGCCTGGTCACCAGGGCTGCT- TAMRA 3′ (SEQ ID NO: 275) where JOE (PE-Applied Biosystems, Foster City, Calif.) is the fluorescent reporter dye) and TAMRA (PE-Applied Biosystems, Foster City, Calif.) is the quencher dye.

### Example 15

Antisense inhibition of human mdm2 expression by chimeric phosphorothioate oligonucleotides having 2′-MOE wings and a deoxy gap

In accordance with the present invention, a series of oligonucleotides were designed to target different regions of the human mdm2 RNA, using published sequences (GenBank accession number Z12020, incorporated herein as SEQ ID NO: 1). The oligonucleotides are shown in Table 13. “Target site” indicates the first (5′-most) nucleotide number on the particular target sequence to which the oligonucleotide binds. All compounds in Table 13 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human mdm2 mRNA levels by quantitative real-time PCR as described in other examples herein. Data are averages from two experiments. If present, “N.D.” indicates “no data”.

TABLE 13

Inhibition of human mdm2 mRNA levels by chimeric
phosphorothioate oligonucleotides having 2′-MOE
wings and a deoxy gap

| | | | | | |
|---|---|---|---|---|---|
| | | SEQ | RE- | TAR- | % |
| | NUCLEOTIDE SEQUENCE | ID | | GET | IN- |
| ISIS # | (5′→3′) | NO | GION | SITE | HIB |

| | | | | | |
|---|---|---|---|---|---|
| 31473 | CAGCCAAGCTCGCGCGGTGC | 3 | 5′ UTR | 1 | 18 |
| 31474 | ACACACAGGGCCACACAGGC | 36 | 5′ UTR | 29 | 13 |
| 31475 | CCCTCGGGCTCGGCTTCTTG | 40 | 5′ UTR | 62 | 36 |
| 31476 | TGCTCCTGGCTGCGAAAGCA | 44 | 5′ UTR | 113 | 33 |
| 31477 | TCTCCGGGCCAGGGCACTGG | 48 | 5′ UTR | 165 | 38 |
| 31478 | GGGACTGACTACTGCGGAAG | 52 | 5′ UTR | 217 | 0 |
| 31479 | TTTGCCTGCTCCTCACCATC | 55 | AUG | 293 | 49 |
| 31480 | ATTTGCCTGCTCCTCACCAT | 56 | AUG | 294 | 1 |
| 31481 | CATTTGCCTGCTCCTCACCA | 9 | AUG | 295 | 36 |
| 31482 | ACATTTGCCTGCTCCTCACC | 57 | AUG | 296 | 44 |
| 31483 | CACATTTGCCTGCTCCTCAC | 58 | AUG | 297 | 28 |
| 31484 | GCACATTTGCCTGCTCCTCA | 59 | AUG | 298 | 61 |
| 31485 | TGCACATTTGCCTGCTCCTC | 60 | AUG | 299 | 84 |
| 31486 | TTGCACATTTGCCTGCTCCT | 61 | AUG | 300 | 77 |
| 31487 | ATTGCACATTTGCCTGCTCC | 62 | AUG | 301 | 79 |
| 31488 | TATTGCACATTTGCCTGCTC | 63 | AUG | 302 | 0 |
| 31489 | GTATTGCACATTTGCCTGCT | 10 | AUG | 303 | 79 |
| 31490 | GGTATTGCACATTTGCCTGC | 64 | AUG | 304 | 86 |
| 31491 | TGGTATTGCACATTTGCCTG | 65 | AUG | 305 | 0 |
| 31492 | TTGGTATTGCACATTTGCCT | 66 | AUG | 306 | 85 |
| 31493 | GTTGGTATTGCACATTTGCC | 67 | AUG | 307 | 91 |
| 31494 | TGTTGGTATTGCACATTTGC | 68 | AUG | 308 | 90 |
| 31495 | ATGTTGGTATTGCACATTTG | 69 | AUG | 309 | 76 |
| 31496 | CATGTTGGTATTGCACATTT | 70 | AUG | 310 | 74 |
| 31497 | ACATGTTGGTATTGCACATT | 71 | AUG | 311 | 59 |
| 31498 | AGACATGTTGGTATTGCACA | 72 | AUG | 313 | 78 |
| 31499 | CAGACATGTTGGTATTGCAC | 73 | AUG | 314 | 84 |
| 31500 | GGAATCTGTGAGGTGGTTAC | 74 | Coding | 351 | 79 |
| 31501 | GCTTTGGTCTAACCAGGGTC | 77 | Coding | 386 | 89 |
| 31502 | GTGCACCAACAGACTTTAAT | 80 | Coding | 422 | 78 |
| 31503 | TAATCGTTTAGTCATAATAT | 83 | Coding | 490 | 24 |
| 31504 | GAACAATATACAATATGTTG | 86 | Coding | 525 | 59 |
| 31505 | TGGCACGCCAAACAAATCTC | 90 | Coding | 559 | 80 |
| 31506 | TATATATTTTCCTGTGCTCT | 94 | Coding | 593 | 0 |
| 31507 | TTCCTGCTGATTGACTACTA | 98 | Coding | 634 | 63 |
| 31508 | CTGTTCTCACTCACAGATGT | 102 | Coding | 669 | 50 |
| 31509 | GGTCCTTTTGATCACTCCCA | 105 | Coding | 704 | 62 |
| 31510 | AAGATGAAGGTTTCTCTTCC | 108 | Coding | 740 | 15 |
| 31511 | CTAGATGAGGTAGATGGTCT | 111 | Coding | 774 | 64 |
| 31512 | TCTGAATTTTCTTCTGTCTC | 114 | Coding | 810 | 61 |
| 31513 | GTGGCGTTTTCTTTGTCGTT | 117 | Coding | 844 | 67 |
| 31514 | GCTTTCATCAAAGGAAAGGG | 120 | Coding | 880 | 58 |
| 31515 | TCACAACATATCTCCCTTAT | 123 | Coding | 915 | 59 |
| 31516 | TGGCGTCCCTGTAGATTCAC | 126 | Coding | 949 | 43 |
| 31517 | GTTCACTTACACCAGCATCA | 129 | Coding | 983 | 63 |
| 31518 | GAAACTGAATCCTGATCCAA | 132 | Coding | 1017 | 55 |
| 31519 | AGATTCAACTTCAAATTCTA | 139 | Coding | 1051 | 25 |
| 31520 | TTCTTCACTAAGGCTATAAT | 143 | Coding | 1084 | 32 |
| 31521 | CCTCATCATCTTCATCTGAG | 147 | Coding | 1115 | 74 |
| 31522 | CTCTCCCCTGCCTGATACAC | 150 | Coding | 1149 | 0 |
| 31523 | AAATTTCAGGATCTTCTTCA | 153 | Coding | 1184 | 17 |
| 31524 | CATTGCATGAAGTGCATTTC | 156 | Coding | 1220 | 69 |
| 31525 | TTCTCACGAAGGGCCCAACA | 160 | Coding | 1275 | 82 |
| 31526 | TCCCCTTTATCTTTCCCTTT | 164 | Coding | 1311 | 11 |
| 31527 | GTGTTGAGTTTTCCAGTTTG | 167 | Coding | 1346 | 59 |
| 31528 | GAATCATTCACTATAGTTTT | 170 | Coding | 1401 | 0 |
| 31529 | TTATCATCATTTTCCTCAAC | 173 | Coding | 1434 | 53 |
| 31530 | CACTTTCTTGTGATTGTGAA | 177 | Coding | 1466 | 48 |
| 31531 | TGCTACTAGAAGTTGATGGC | 180 | Coding | 1499 | 66 |
| 31532 | AACTCTTTCACATCTTCTTG | 184 | Coding | 1533 | 61 |
| 31533 | CTCTCTTCTTTGTCTTGGGT | 188 | Coding | 1566 | 68 |
| 31534 | TGACAAATCACACAAGGTTC | 191 | Coding | 1617 | 74 |
| 31535 | TAAGATGTCCTGTTTTGCCA | 195 | Coding | 1667 | 8 |
| 31536 | TTTCTTTAGCTTCTTTGCAC | 199 | Coding | 1702 | 67 |
| 31537 | TTGGTTGTCTACATACTGGG | 203 | Coding | 1736 | 66 |
| 31538 | AAATATATAATTCTCTTATA | 207 | 3′ UTR | 1798 | 0 |
| 31539 | CAGGTTGTCTAAATTCCTAG | 212 | 3′ UTR | 1832 | 85 |
| 31540 | CATTTTCTCACTTTGATATA | 216 | 3′ UTR | 1865 | 51 |
| 31541 | ATACTAAAGAGAAGAAATCT | 220 | 3′ UTR | 1898 | 0 |
| 31542 | AGTATTCACTATTCCACTAC | 225 | 3′ UTR | 1933 | 71 |
| 31543 | AAAGGATGAGCTACATATTC | 229 | 3′ UTR | 1969 | 0 |
| 31544 | CAGAGTAGAAATTATTTAAA | 234 | 3′ UTR | 2004 | 20 |
| 31545 | TTAAATGTCATATACATATT | 238 | 3′ UTR | 2059 | 3 |
| 31546 | CAGCCTGGGTAACAGAGCAA | 241 | 3′ UTR | 2111 | 64 |
| 31547 | GAGCTTGCAGTGAGCCAAGA | 246 | 3′ UTR | 2146 | 42 |
| 31548 | CAAGCTAATTGGGAGGCTGA | 251 | 3′ UTR | 2198 | 48 |
| 31549 | AATTAGCCAGGTGTGGTGGC | 256 | 3′ UTR | 2232 | 77 |
| 31550 | AGACCATCCTGGCTAACACG | 260 | 3′ UTR | 2283 | 0 |
| 31551 | AGGCCGAGGTGGGCGGATCA | 264 | 3′ UTR | 2319 | 2 |

As shown in Table 13, SEQ ID NOs 10, 59, 60, 61, 62, 64, 66, 67, 68, 59, 70, 72, 73, 74, 77, 80, 90, 98, 105, 111, 114, 117, 129, 147, 156, 160, 180, 184, 188, 191, 199, 203, 212, 225, 241 and 256 demonstrated at least 60% inhibition of human mdm2 expression in this assay and are therefore preferred. The target sites to which these preferred sequences are complementary are herein referred to as “active sites” and are therefore preferred sites for targeting by compounds of the present invention.

### Example 16

Inhibition of human mdm2 expression by additional chimeric phosphorothioate oligonucleotides having 2′-MOE wings and a deoxy gap

In accordance with the present invention, a second series of oligonucleotides were designed to target additional regions of the human mdm2 RNA, using published sequences (GenBank accession number Z12020, incorporated herein as SEQ ID NO: 1). The oligonucleotides are shown in Table 14. “Target site” indicates the first (5′-most) nucleotide number on the particular target sequence to which the oligonucleotide binds. All compounds in Table 14 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human mdm2 mRNA levels by quantitative real-time PCR as described in other examples herein. Data are averages from two experiments. If present, “N.D.” indicates “no data”.

TABLE 14

Inhibition of human mdm2 mRNA levels by chimeric
phosphorothioate oligonucleotides having 2′-MOE
wings and a deoxy gap

| | | | | | |
|---|---|---|---|---|---|
| | | SEQ | | TAR- | |
| | NUCLEOTIDE SEQUENCE | ID | REG- | GET | & |
| ISIS # | (5′→3′) | NO | ION | SITE | INHIB |

| | | | | | |
|---|---|---|---|---|---|
| 31632 | CAGGCCCCAGAAGCAGCCAA | 34 | 5′ UTR | 14 | 0 |
| 31633 | GCTCCATCTTTCCGACACAC | 38 | 5′ UTR | 43 | 39 |
| 31634 | AAGCAGCAGGATCTCGGTCA | 42 | 5′ UTR | 98 | 55 |
| 31635 | ACTGGGCGCTCGTACGCACT | 46 | 5′ UTR | 150 | 23 |
| 31636 | GGAAGCACGACGCCCTGGGC | 50 | 5′ UTR | 202 | 6 |
| 31637 | CCTGCTCCTCACCATCCGGG | 54 | 5′ UTR | 289 | 57 |
| 31638 | CAGTAGGTACAGACATGTTG | 75 | Coding | 323 | 69 |
| 31639 | TTCCGAAGCTGGAATCTGTG | 78 | Coding | 361 | 71 |
| 31640 | GCAATGGCTTTGGTCTAACC | 81 | Coding | 392 | 54 |
| 31641 | ACCTCTTTCATAGTATAAGT | 84 | Coding | 450 | 56 |
| 31642 | GCTTCTCATCATATAATCGT | 88 | Coding | 503 | 72 |
| 31643 | TAGAAGATCATTTGAACAAT | 92 | Coding | 538 | 34 |
| 31644 | CTTTCACAGAGAAGCTTGGC | 96 | Coding | 575 | 43 |
| 31645 | TTCCTGTAGATCATGGTATA | 100 | Coding | 609 | 24 |
| 31646 | TGAGTCCGATGATTCCTGCT | 103 | Coding | 646 | 61 |
| 31647 | TTCAAGGTGACACCTGTTCT | 106 | Coding | 682 | 40 |
| 31648 | AAGCTCTTGTACAAGGTCCT | 109 | Coding | 718 | 68 |
| 31649 | AAACCAAATGTGAAGATGAA | 112 | Coding | 752 | 0 |
| 31650 | AATTGCTCTCCTTCTAGATG | 115 | Coding | 787 | 20 |
| 31651 | CACCAGATAATTCATCTGAA | 118 | Coding | 824 | 82 |
| 31652 | TACTATCAGATTTGTGGCGT | 121 | Coding | 857 | 45 |
| 31653 | TACACACAGAGCCAGGCTTT | 124 | Coding | 895 | 58 |
| 31654 | CTACTGCTTCTTTCACAACA | 127 | Coding | 927 | 63 |
| 31655 | AAGATCCGGATTCGATGGCG | 130 | Coding | 964 | 77 |
| 31656 | CAATCACCTGAATGTTCACT | 133 | Coding | 996 | 10 |
| 31657 | CTACACTAAACTGATCTGAA | 137 | Coding | 1034 | 70 |
| 31658 | TAATCTTCTGAGTCGAGAGA | 141 | Coding | 1068 | 30 |
| 31659 | CTGAGAGTTCTTGTCCTTCT | 145 | Coding | 1100 | 81 |
| 31660 | GAATCTGTATCACTCTCCCC | 151 | Coding | 1161 | 82 |
| 31661 | AGTCAGCTAAGGAAATTTCA | 154 | Coding | 1196 | 42 |
| 31662 | CATCTGTTGCAATGTGATGG | 158 | Coding | 1257 | 55 |
| 31663 | TATCTTCAGGAAGCCAATTC | 162 | Coding | 1292 | 0 |
| 31664 | CTTTCTCAGAGATTTCCCCT | 165 | Coding | 1325 | 48 |
| 31665 | CCTCTTCAGCTTGTGTTGAG | 168 | Coding | 1358 | 19 |
| 31666 | ATGACTCTCTGGAATCATTC | 171 | Coding | 1412 | 81 |
| 31667 | GAAGCTTGTGTAATTTTATC | 175 | Coding | 1449 | 43 |
| 31668 | GCTGAGAATAGTCTTCACTT | 178 | Coding | 1481 | 50 |
| 31669 | CTTGGCTGCTATAAATAATG | 182 | Coding | 1517 | 55 |
| 31670 | ATCTTCTTGGCTGCTATAAA | 183 | Coding | 1522 | 51 |
| 31671 | GGGTTTCTTCCCTTTCAAAC | 186 | Coding | 1550 | 62 |
| 31672 | AACTAGATTCCACACTCTCT | 189 | Coding | 1580 | 63 |
| 31673 | TCGACCTTGACAAATCACAC | 192 | Coding | 1624 | 67 |
| 31674 | ATGGACAATGCAACCATTTT | 193 | Coding | 1648 | 55 |
| 31675 | GCAGGCCATAAGATGTCCTG | 196 | Coding | 1675 | 67 |
| 31676 | ACATGTAAAGCAGGCCATAA | 197 | Coding | 1684 | 48 |
| 31677 | TTATTCCTTTTCTTTAGCTT | 200 | Coding | 1710 | 65 |
| 31678 | TGGGCAGGGCTTATTCCTTT | 201 | Coding | 1720 | 49 |
| 31679 | TCATTTGAATTGGTTGTCTA | 204 | Coding | 1745 | 35 |
| 31680 | AAGTTAGCACAATCATTTGA | 205 | Coding | 1757 | 34 |
| 31681 | TCTCTTATAGACAGGTCAAC | 206 | STOP | 1787 | 78 |
| | | | COD- | | |
| | | | ON | | |
| 31682 | AGTTAGAAATATATAATTCT | 208 | 3′ UTR | 1804 | 0 |
| 31683 | CTAGGGTTATATAGTTAGAA | 210 | 3′ UTR | 1816 | 70 |
| 31684 | ATAAATTTCAGGTTGTCTAA | 213 | 3′ UTR | 1840 | 16 |
| 31685 | ATATATGTGAATAAATTTCA | 214 | 3′ UTR | 1850 | 0 |
| 31686 | CTTTGATATATGTGAATAAA | 215 | 3′ UTR | 1855 | 56 |
| 31687 | ATTGAGGCATTTTCTCACTT | 217 | 3′ UTR | 1872 | 14 |
| 31688 | AATCTATGTGAATTGAGGCA | 218 | 3′ UTR | 1883 | 73 |
| 31689 | AGAAGAAATCTATGTGAATT | 219 | 3′ UTR | 1889 | 33 |
| 31690 | GTCAATTATACTAAAGAGAA | 221 | 3′ UTR | 1905 | 44 |
| 31691 | CAAAGTAGGTCAATTATACT | 223 | 3′ UTR | 1913 | 8 |
| 31692 | TATAGTAAGTATTCACTATT | 226 | 3′ UTR | 1940 | 4 |
| 31693 | AGTCAAATTATAGTAAGTAT | 227 | 3′ UTR | 1948 | 24 |
| 31694 | TAGGAGTTGGTGTAAAGGAT | 231 | 3′ UTR | 1982 | 65 |
| 31695 | GAAATTATTTAAAATTAGGA | 233 | 3′ UTR | 1997 | 17 |
| 31696 | CTCATTTAAGACAGAGTAGA | 235 | 3′ UTR | 2015 | 75 |
| 31697 | CATATACATATTTAAGAAAA | 237 | 3′ UTR | 2051 | 0 |
| 31698 | TAATAAGTTACATTTAAATG | 239 | 3′ UTR | 2072 | 0 |
| 31699 | GTAACAGAGCAAGACTCGGT | 240 | 3′ UTR | 2103 | 31 |
| 31700 | CCCACTGCACTCCAGCCTGG | 243 | 3′ UTR | 2123 | 63 |
| 31701 | GCAGTGAGCCAAGATCACCC | 245 | 3′ UTR | 2140 | 52 |
| 31702 | CAGGAGAATGGTGCGAACCC | 248 | 3′ UTR | 2176 | 0 |
| 31703 | AGGCTGAGGCAGGAGAATGG | 249 | 3′ UTR | 2185 | 57 |
| 31704 | AGGCCAAGCTAATTGGGAGG | 252 | 3′ UTR | 2202 | 0 |
| 31705 | CAGATGACTGTAGGCCAAGC | 254 | 3′ UTR | 2213 | 48 |
| 31706 | AGGTGTGGTGGCAGATGACT | 21 | 3′ UTR | 2224 | 38 |
| 31707 | GTCTCTACTAAAAGTACAAA | 257 | 3′ UTR | 2253 | 28 |
| 31708 | CGGTGAAACCCTGTCTCTAC | 258 | 3′ UTR | 2265 | 70 |
| 31709 | GAGGTCAGGAGATCGAGACC | 262 | 3′ UTR | 2298 | 0 |
| 31710 | TCCCAGCACTTTGGGAGGCC | 266 | 3′ UTR | 2334 | 27 |
| 31711 | GTGGCTCATGCCTGTAATCC | 268 | 3′ UTR | 2351 | 54 |

As shown in Table 14, SEQ ID NOs 42, 54, 75, 78, 81, 84, 88, 96, 103, 106, 109, 118, 121, 124, 127, 130, 137, 145, 151, 154, 158, 165, 171, 175, 178, 182, 183, 186, 189, 192, 193, 196, 197, 200, 201, 206, 210, 215, 218, 221, 231, 235, 243, 245, 249, 254, 258 and 268 demonstrated at least 40% inhibition of human mdm2 expression in this assay and are therefore preferred. The target sites to which these preferred sequences are complementary are herein referred to as “active sites” and are therefore preferred sites for targeting by compounds of the present invention.

### Example 17

Additional Human mdm2 Antisense Oligonucleotides

In accordance with the present invention, additional oligonucleotides were designed to target regions of the human mdm2 RNA, using published sequences (GenBank accession number Z12020, incorporated herein as SEQ ID NO: 1). The oligonucleotides are shown in Table 15. “Target site” indicates the first (5′-most) nucleotide number on the particular target sequence to which the oligonucleotide binds. All compounds in Table 15 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine resides are 5-methylcytidines.

TABLE 15

Nucleotide Sequence of Human mdm2 chimeric phos-
phorothioate oligonucleotides having 2′-MOE wings
and a deoxy gap

| | | | | |
|---|---|---|---|---|
| | NUCLEOTIDE SEQUENCE SEQ ID | | TARGET | |
| ISIS # | (5′→3′) | NO | REGION | SITE |

| | | | | |
|---|---|---|---|---|
| 108679 | ACAGACATGTTGGTATTGCA | 276 | Coding | 315 |
| 108680 | AAGCTGGAATCTGTGAGGTG | 277 | Coding | 356 |
| 108681 | GAAGCTGGAATCTGTGAGGT | 278 | Coding | 357 |
| 108682 | CGAAGCTGGAATCTGTGAGG | 279 | Coding | 358 |
| 108683 | CCGAAGCTGGAATCTGTGAG | 280 | Coding | 359 |
| 108684 | TCCGAAGCTGGAATCTGTGA | 281 | Coding | 360 |
| 108685 | GTTCCGAAGCTGGAATCTGT | 282 | Coding | 362 |
| 108686 | TGTTCCGAAGCTGGAATCTG | 283 | Coding | 363 |
| 108687 | TTGTTCCGAAGCTGGAATCT | 284 | Coding | 364 |
| 108688 | CTTGTTCCGAAGCTGGAATC | 285 | Coding | 365 |
| 108689 | TCTTGTTCCGAAGCTGGAAT | 286 | Coding | 366 |
| 108690 | CTCTTGTTCCGAAGCTGGAA | 287 | Coding | 367 |
| 108691 | TCTCTTGTTCCGAAGCTGGA | 288 | Coding | 368 |
| 108692 | GTCTCTTGTTCCGAAGCTGG | 289 | Coding | 369 |
| 108693 | AGTCATAATATACTGGCCAA | 290 | Coding | 481 |
| 108694 | TAGTCATAATATACTGGCCA | 291 | Coding | 482 |
| 108695 | TTAGTCATAATATACTGGCC | 292 | Coding | 483 |
| 108696 | CTCCTTCTAGATGAGGTAGA | 293 | Coding | 780 |
| 108697 | TCTCCTTCTAGATGAGGTAG | 294 | Coding | 781 |
| 108698 | CAATAGTCAGCTAAGGAAAT | 295 | Coding | 1200 |
| 108699 | CCAATAGTCAGCTAAGGAAA | 296 | Coding | 1201 |
| 108700 | TCCAATAGTCAGCTAAGGAA | 297 | Coding | 1202 |
| 108701 | TTCCAATAGTCAGCTAAGGA | 298 | Coding | 1203 |
| 108702 | GGATTCATTTCATTGCATGA | 299 | Coding | 1230 |
| 108703 | GAGTTTTCCAGTTTGGCTTT | 300 | Coding | 1341 |
| 108704 | TGAGTTTTCCAGTTTGGCTT | 301 | Coding | 1342 |

### Example 18

Additional Human mdm2 Antisense Oligonucleotides containing a larger central gap region

In accordance with the present invention, additional olignucleotides were designed to target regions of the human mdm2 RNA, using published sequences (GenBank accession number Z12020, incorporated herein as SEQ ID NO: 1). The oligonucleotides are shown in Table 16. “Target site” indicates the first (5′-most) nucleotide number on the particular target sequence to which the oligonucleotide binds. All compounds in Table 16 are chimeric oligonucleotides (“gapmers”) 20 nucleotides in length, composed of a central “gap” region consisting of twelve 2′-deoxynucleotides, deoxynucleotides, which is flanked on both sides (5′ and 3′ directions) by four-nucleotide “wings”. The wings are composed of 2′-methoxyethyl (2′-MOE)nucleotides. The internucleoside (backbone) linkages are phosphorothioate (P═S) throughout the oligonucleotide. All cytidine resides are 5-methylcytidines.

TABLE 16

Nucleotide Sequence of Human mdm2 chimeric phos-
phorothioate oligonucleotides having 2′-MOE wings
and a larger deoxy gap

| | | | |
|---|---|---|---|
| | NUCLEOTIDE SEQUENCE SEQ ID | | TARGET |
| ISIS # | (5′→3′) NO REGION | SITE | |

| | | | | |
|---|---|---|---|---|
| 116425 | GACCTTGACAAATCACACAA | 302 | Coding | 1622 |
| 116426 | TTTTTAGGTCGACCTTGACA | 303 | Coding | 1632 |
| 116427 | AATGCAACCATTTTTAGGTC | 304 | Coding | 1642 |
| 116428 | TGCCATGGACAATGCAACCA | 305 | Coding | 1652 |
| 116429 | TGTCCTGTTTTGCCATGGAC | 306 | Coding | 1662 |
| 116430 | GGCCATAAGATGTCCTGTTT | 307 | Coding | 1672 |
| 116431 | ATGTAAAGCAGGCCATAAGA | 308 | Coding | 1682 |
| 116432 | TTCTTTGCACATGTAAAGCA | 309 | Coding | 1692 |
| 116433 | GCTTATTCCTTTTCTTTAGC | 310 | Coding | 1712 |
| 116434 | ACTGGGCAGGGCTTATTCCT | 311 | Coding | 1722 |
| 116435 | TTGTCTACATACTGGGCAGG | 312 | Coding | 1732 |
| 116436 | TTTGAATTGGTTGTCTACAT | 313 | Coding | 1742 |
| 116437 | AGCACAATCATTTGAATTGG | 314 | Coding | 1752 |
| 116438 | GAAATAAGTTAGCACAATCA | 315 | Coding | 1762 |
| | | | STOP | |
| 116439 | TCAACTAGGGGAAATAAGTT 316 | CODON | 1772 | |
| | | | STOP | |
| 116440 | TATAGACAGGTCAACTAGGG | 317 | CODON | 1782 |
| 116441 | ATAATTCTCTTATAGACAGG | 318 | 3′ UTR | 1792 |

### Example 19

Oligonucleotides designed to nucleotides 1695-1714 of Human mdm2-Modifications to “gap” placement

In accordance with the present invention, oligonucleotides containing several chemical modifications, were designed to target nucleotides 1695-1714 of Human mdm2 (Genbank accession NO: Z12020, incorporated herein as SEQ ID NO 1). These modifications are described in this and following examples.

The oligonucleotides shown in Table 17 are chimeric oligonucleotides (“gapmers”) 20 nucleotides in length, composed of a central “gap” region flanked on both sides (5′ and 3′ directions) by nucleotide “wings” represented by bolded nucleotides. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. “Target site” indicates the first (5′-most) nucleotide number on the particular target sequence to which the oligonucleotide binds.

TABLE 17

Chimeric phosphorothioate antisense oligonucleo-
tides designed to nucleotides 1695-1714 of Human
mdm2

| | | | | |
|---|---|---|---|---|
| | NUCLEOTIDE SEQUENCE | SEQ ID | | TARGET |
| ISIS # | (5′→3′) | NO | REGION | SITE |

| | | | | |
|---|---|---|---|---|
| 104630 | AGCTTCTTTGCACATGTAAA | 15 | Coding | 1695 |
| 105271 | AGCTTCTTTGCACATGTAAA | 15 | Coding | 1695 |
| 107909 | AGCTTCTTTGCACATGTAAA | 15 | Coding | 1695 |
| 107910 | AGCTTCTTTGCACATGTAAA | 15 | Coding | 1695 |
| 107930 | AGCTTCTTTGCACATGTAAA | 15 | Coding | 1695 |
| 107931 | AGCTTCTTTGCACATGTAAA | 15 | Coding | 1695 |
| 107932 | AGCTTCTTTGCACATGTAAA | 15 | Coding | 1695 |
| 108494 | AGCTTCTTTGCACATGTAAA | 15 | Coding | 1695 |
| 134040 | AGCTTCTTTGCACATGTAAA | 15 | Coding | 1695 |

Four oligonucleotides in Table 17 were tested for their ability to reduce mdm2 mRNA expression in A549 cells. Cells were treated at doses of 30, 100, 200 and 400 nM and 5 mRNA levels were measured by RT-PCR as described in other examples herein. The data were compared to the previously identified lead, ISIS 16518. All were capable of reducing the expression of Human mdm2 mRNA at the lowest dose, except ISIS 107932. The data are shown in Table 18.

TABLE 18

Inhibition of Human mdm2 mRNA expression by chime-
ric phosphorothioate antisense oligonucleotides
with varying gap size and gap placement

| | | | | | |
|---|---|---|---|---|---|
| | | % | % | % | |
| | | In- | In- | In- | % |
| | | hib. | hib. | hib. | Inhib. |
| | NUCLEOTIDE SEQUENCE | (30 | (100 | (200 | (400 |
| ISIS # | (5′→3′) | nM) | nM) | nM) | nM) |

| | | | | | |
|---|---|---|---|---|---|
| 16518 | AGCTTCTTTGCACATGTAAA | 45 | 82 | 90 | 93 |
| 105271 | AGCTTCTTTGCACATGTAAA | 68 | 95 | 98 | 99 |
| 107910 | AGCTTCTTTGCACATGTAAA | 45 | 83 | 95 | 97 |
| 107931 | AGCTTCTTTGCACATGTAAA | 54 | 85 | 93 | 97 |
| 107932 | AGCTTCTTTGCACATGTAAA | 0 | 42 | 77 | 88 |

### Example 20

Oligonucleotides designed to nucleotides 1695-1714 of Human mdm2-Modifications to the sugar

The oligonucleotides shown in Table 19 are chimeric oligonucleotides (“gapmers”) 20 nucleotides in length, composed of a central “gap” region flanked on both sides (5′ and 3′ directions) by nucleotide “wings”. The nucleotide wings are composed of one or more sugar modifications including 2′-methoxyethyl (2′-MOE), 2′-O-methylribose, 2′-O-propylribose, 2′-O-[(N-palmityl)-6-aminohexyl] ribose, 2′-O-[(4-isobutylphenyl) isopropionylaminohexyl] ribose, 2′-O-dimethylaminooxyethyl (DMAOE) ribose or 2′-O-N-[2-(dimethylamino)ethyl]acetamido ribose. The internucleoside (backbone) linkages are phosphorothioate (P═S) throughout the oligonucleotides. All cytidine residues are 5-methylcytidines unless noted. All sequences have SEQ ID NO: 15.

TABLE 19

Antisense Oligonucleotides with sugar modifications

| | | | |
|---|---|---|---|
| | | | Sugar |
| | NUCLEOTIDE SEQUENCE | | Modification |
| ISIS # | (5′→3′) | Sugar Modification | Position |

| | | | |
|---|---|---|---|
| 32393 | AGCTTCTTTGCACATGTAAA | 2′-O-methylribose | 1,2,19,20 |
| 108495 | AGCTTCTTTGCACATGTAAA* | 2′-O-methylribose | 1-5; 16-20 |
| 108496 | AGCTTCTTTGCACATGTAAA* | 2′-O-propylribose | 1-5; 16-20 |
| 111496 | AGCTTCTTTGCACATGTAAA | 2′-methoxyethyl | 1-5; 16-19 |
| | | (2′-MOE) ribose | |
| | | 2′-O-[(4- | 20 |
| | | isobutylphenyl) isopropionylaminohexyl] | |
| | | ribose | |
| 111497 | AGCTTCTTTGCACATGTAAA | 2′-methoxyethyl | 1-5; 16-19 |
| (2′-MOE) ribose | | | |
| | | 2′-O-[(4- | 20 |
| | | isobutylphenyl) isopropionylaminohexyl] | |
| | | ribose | |
| 121645 | AGCTTCTTTGCACATGTAAA | DMAOE | 1-5; 16-20 |
| 123190 | AGCTTCTTTGCACATGTAAA | 2′-methoxyethyl 3-5; 16-18 | |
| | | (2′-MOE) ribose | |
| | | 2′-O-N-[2- | 1,2; 19,20 |
| | | (dimethylamino) ethyl] | |
| | | acetamido ribose | |

*ISIS 108495 and ISIS 108496 have cytosine residues at position 3.

### Example 21

Oligonucleotides designed to nucleotides 1695-1714 of Human mdm2-Modifications to the linker

The oligonucleotides shown in Table 20 are chimeric oligonucleotides (“gapmers”) 20 nucleotides in length, composed of an eight 2′-deoxynucleotide central “gap” region flanked on both sides (5′ and 3′ directions) by six-nucleotide “wings”. The wings are composed of 2′-methoxyethyl (2′-MOE)nucleotides. The internucleoside (backbone) linkages are phosphorothioate (P═S) or phosphate esters. Phosphate ester linkages are noted in bold and are in the 5′ to 3′ direction throughout the oligonucleotide. Consequently, there is no linker on the final nucleotide. All cytidine residues are 5-methylcytidines. All sequences have SEQ ID NO: 15.

TABLE 20

Antisense Oligonucleotides with phosphate ester
linkage modifications

| | |
|---|---|
| | NUCLEOTIDE SEQUENCE |
| ISIS # | (′→3′) |

| | |
|---|---|
| 119186 | AGCTTCTTTGCACATGTAAA |
| 119187 | AGCTTCTTTGCACATGTAAA |
| 119188 | AGCTTCTTTGCACATGTAAA |
| 119189 | AGCTTCTTTGCACATGTAAA |
| 119190 | AGCTTCTTTGCACATGTAAA |
| 119191 | AGCTTCTTTGCACATGTAAA |

### Example 22

Oligonucleotides designed to nucleotides 1695-1714 of Human mdm2-Modifications to the heterocycle

The oligonucleotides shown in Table 21 are phosphorothioate oligonucleotides 20 nucleotides in length. Certain oligonucleotides are composed of a ten 2′-deoxynucleotide central “gap” region flanked on both sides (5′ and 3′ directions) by five-nucleotide “wings”. The wings are composed of 2′-methoxyethyl(2′-MOE)nucleotides and are shown in bold. All other nucleotides are 2′deoxyribose throughout the oligonucleotide.

The internucleoside (backbone) linkages are phosphorothioate throughout the oligonucleotides. As noted in Table 20, certain cytosines have been replaced with the cytosine derivative, 1,3-diazaphenoxazine-2-one (G-clamp). All other cytidine residues are 5-methylcytidines. All sequences have SEQ ID NO: 15.

TABLE 21

Antisense Oligonucleotides with heterocycle modi-
fications-G Clamps

| | | | |
|---|---|---|---|
| | | Hetero- | |
| | | cycle | Heterocycle |
| | NUCLEOTIDE SEQUENCE | Modifi- | Modification |
| ISIS # | (5′→3′) | cation | Position |

| | | | |
|---|---|---|---|
| 109712 | AGCTTCTTTGCACATGTAAA | G-clamp | 3 |
| 109713 | AGCTTCTTTGCACATGTAAA | G-clamp | 6 |
| 109714 | AGCTTCTTTGCACATGTAAA | G-clamp | 11 |
| 109715 | AGCTTCTTTGCACATGTAAA | C-clamp | 13 |
| 109716 | AGCTTCTTTGCACATGTAAA | C-clamp | 3, 6 |
| 109717 | AGCTTCTTTGCACATGTAAA | C-clamp | 11, 13 |
| 109718 | AGCTTCTTTGCACATGTAAA | C-clamp | 6 |
| 109719 | AGCTTCTTTCCACATGTAAA | G-clamp | 11 |
| 109720 | AGCTTCTTTGCACATGTAAA | G-clamp | 13 |
| 109721 | AGCTTCTTTGCACATGTAAA | C-clamp | 6, 13 |
| 119427 | AGCTTCTTTGCACATGTAAA | G-clamp | 3 |
| 119428 | AGCTTCTTTGCACATGTAAA | G-clamp | 3, 11 |
| 119465 | AGCTTCTTTGCACATGTAAA | G-clamp | 3, 13 |

In a further embodiment of the invention, A549 cells were treated with ISIS 119427 and ISIS 119465 at doses of 10, 30, 100 and 300 nM and the level of Human mdm2 mRNA was measured by RT-PCR as described in other examples herein. The results are compared to ISIS 16518 and ISIS 121645, described previously. The data are shown in Table 22.

TABLE 22
Inhibition of Human mdm2 mRNA expression by chimeric
phosphorothioate antisense oligonucleotides with modified
heterocycles

| | % | % | % | % |
|---|---|---|---|---|
| | Inhib. | Inhib. | Inhib. | Inhib. |
| ISIS # | (10 nM) | (30 nM) | (100 nM) | (300 nM) |
| 16518 | 25 | 70 | 84 | 99 |
| 121645 | 32 | 60 | 82 | 97 |
| 119427 | 35 | 70 | 87 | 98 |
| 119465 | 35 | 75 | 97 | 100 |

### Example 23

Oligonucleotides designed to nucleotides 1695-1714 of Human mdm2-Additional Modifications to the heterocycle

In accordance with the present invention, a second series of oligonucleotides were designed with modifications to the heterocycle base. The oligonucleotides are shown in Table 23. ISIS 109728-109731, ISIS 11629, ISIS 121646 and ISIS 142960 are chimeric oligonucleotides (“gapmers”) 20 nucleotides in length, composed of a central “gap” region consisting of 2′-deoxynucleotides, which is flanked on both sides (5′ and 3′ directions) by nucleotide “wings”. The wings are composed of 2′-methoxyethyl (2′-MOE)nucleotides and are shown in bolded text. ISIS 109722-109727 are phosporothioate oligonucleotides composed only of 2′-deoxynucleotides. The internucleoside (backbone) linkages are phosphorothioate (P═S) throughout all of the olignucleotides. Select cytidine residues have been modified to 5-methylcytidine and these positions are noted in the table. All sequences have SEQ ID NO: 15.

TABLE 23

Phosphorothioate antisense oligonucleotides con-
taining modifications to cytidine

| | | | |
|---|---|---|---|
| | | Hetero- | |
| | | cycle | Heterocycle |
| | NUCLEOTIDE SEQUENCE | Modifi- | Modication |
| ISIS # | (5′→3′) | cation | Position |

| | | | |
|---|---|---|---|
| 109722 | AGCTTCTTTGCACATGTAAA | Cytidine | 6, 11, 13 |
| | | to 5- | |
| | | methyl- | |
| | | cytidine | |
| 109723 | AGCTTCTTTGCACATGTAAA | Cytidine | 3, 11, 13 |
| | | to 5- | |
| | | methyl- | |
| | | cytidine | |
| 109724 | AGCTTCTTTGCACATGTAAA | Cytidine | 3, 6, 13 |
| | | to 5- | |
| | | methyl- | |
| | | cytidine | |
| 109725 | AGCTTCTTTGCACATGTAAA | Cytidine | 3, 6, 11 |
| | | to 5- | |
| | | methyl- | |
| | | cytidine | |
| 109726 | AGCTTCTTTGCACATGTAAA | Cytidine | 11, 13 |
| | | to 5- | |
| | | methyl- | |
| | | cytidine | |
| 109727 | AGCTTCTTTGCACATGTAAA | Cytidine | 3, 6 |
| | | to 5- | |
| | | methyl- | |
| | | cytidine | |
| 109728 | AGCTTCTTTGCACATGTAAA | Cytidine | 3, 11, 13 |
| | | to 5- | |
| | | methyl- | |
| | | cytidine | |
| 109729 | AGCTTCTTTGCACATGTAAA | Cytidine | 3, 6, 13 |
| | | to 5- | |
| | | methyl- | |
| | | cytidine | |
| 109730 | AGCTTCTTTGCACATGTAAA | Cytidine | 3, 6, 11 |
| | | to 5- | |
| | | methyl- | |
| | | cytidine | |
| 109731 | AGCTTCTTTGCACATGTAAA | Cytidine | 3, 11 |
| | | to 5- | |
| | | methyl- | |
| | | cytidine | |
| 111629 | AGCTTCTTTGCACATGTAAA | Cytidine | 3 |
| | | to 5- | |
| | | methyl- | |
| | | cytidine | |
| 121646 | AGCTTCTTTGCACATGTAAA | Cytidine | 3, 6 |
| | | to 5- | |
| | | methyl- | |
| | | cytidine | |
| 142960 | AGCTTCTTTGCACATGTAAA | Cytidine | 3, 6 |
| | | to 5- | |
| | | methyl- | |
| | | cytidine | |

### Example 24

Oligonucleotides designed to nucleotides 1695-1714 of Human mdm2-Combinatorial Modifications to the heterocycle

In accordance with the present invention, a series of oligonucleotides were designed with modifications to the heterocycle base. The oligonucleotides are shown in Table 24. ISIS 111175-111178, ISIS 139364 and ISIS 142960 are chimeric oligonucleotides (“gapmers”) 20 nucleotides in length, composed of a central “gap” region consisting of 2′-deoxynucleotides, which is flanked on both sides (5′ and 3′ directions) by nucleotide “wings”. The wings are composed of 2′-methoxyethyl (2′-MOE)nucleotides and are shown in bolded text. ISIS 111169-111174 and ISIS 138702 are phosporothioate oligonucleotides composed only of 2′-deoxynucleotides. The internucleoside (backbone) linkages are phosphorothioate (P═S) throughout all of the oligonucleotides. Select cytidine residues have been modified to 5-methylcytidine and these positions are noted in the table. In addition, certain cytosines have been replaced with the cytosine derivative, 1,3-diazaphenoxazine-2-one (G-clamp) and these are noted in the table. All sequences have SEQ ID NO: 15.

TABLE 24

Phosphorothioate antisense oligonucleotides con-
taining multiple modifications to cytidine

| | | | |
|---|---|---|---|
| | | | 5- |
| | | | methyl- |
| | | G-Clamp | cytidine |
| | | Modifi- | Modifi- |
| | NUCLEOTIDE SEQUENCE | cation | cation |
| ISIS # | (5′→3′) | Position | Position |

| | | | |
|---|---|---|---|
| 111169 | AGCTTCTTTGCACATGTAAA | 3 | none |
| 111170 | AGCTTCTTTGCACATGTAAA | 6 | none |
| 111171 | AGCTTCTTTGCACATGTAAA | 11 | none |
| 111172 | AGCTTCTTTGCACATGTAAA | 13 | none |
| 111173 | AGCTTCTTTGCACATGTAAA | 3, 6 | none |
| 111174 | AGCTTCTTTGCACATGTAAA | 11, 13 | none |
| 138702 | AQCTTCTTTGCACATGTAAA | 3, 13 | none |
| 111175 | AGCTTCTTTGCACATGTAAA | 6 | 3 |
| 111176 | AGCTTCTTTGCACATGTAAA | 11 | 3 |
| 111177 | AGCTTCTTTGCACATGTAAA | 13 | 3 |
| 111178 | AGCTTCTTTGCACATGTAAA | 6, 13 | 3 |
| 139364 | AGCTTCTTTGCACATGTAAA | 3, 6 | none |

### Example 25

Oligonucleotides designed to nucleotides 1695-1714 of Human mdm2-Conjugate modifications to the heterocycle

In accordance with the present invention, a series of oligonucleotides were designed with modifications to the sugar. The oligonucleotides are shown in Table 25. Both oligonucleotides are chimeric oligonucleotides (“gapmers”) 20 nucleotides in length, composed of a central “gap” region consisting of 2′-deoxynucleotides, which is flanked on both sides (5′ and 3′ directions) by nucleotide “wings”. The wings are composed of 2′-methoxyethyl (2′-MOE) nucleotides and are shown in bolded text. The internucleoside (backbone) linkages are phosphorothioate (P═S) throughout the oligonucleotides. Select cytidine residues have been modified to 5-methylcytidine and these positions are noted in the table. The sugar has been modified to 2′-(gamma-Folate) at position four for ISIS 122705 and to 2′-O-taxol at position 20 for ISIS 13427. All sequences have SEQ ID NO: 15.

TABLE 25

Phosphorothioate antisense oligonucleotides con-
taining modifications to the sugar

| | | | |
|---|---|---|---|
| | | | 5-methyl- |
| | | Conjugate | cytidine |
| | NUCLEOTIDE SEQUENCE | and | Modification |
| ISIS · | (5′→3′ | Position | Position |

| | | | |
|---|---|---|---|
| 122705 | AGCTTCTTTGCACATGTAAA | 2′-(gamma- | 3 |
| | | Folate); 4 | |
| 134247 | AGCTTCTTTGCACATGTAAA | 2′-O-taxol; 20 | 3, 6 |

### Example 26

Oligonucleotides designed to nucleotides 1695-1714 of Human mdm2-Propynyl and phenoxazine modifications to the heterocycle

In accordance with the present invention, certain oligonucleotides were designed with modifications to the heterocycle. The oligonucleotides are shown in Table 26. All of the oligonucleotides are chimeric oligonucleotides (“gapmers”) 20 nucleotides in length, composed of a central “gap” region consisting of 2′-deoxynucleotides, which is flanked on both sides (5′ and 3′ directions) by nucleotide “wings”. The wings are composed of 2′-methoxyethyl (2′-MOE)nucleotides and are shown in bolded text. The internucleoside (backbone) linkages are phosphorothioate (P═S) throughout the oligonucleotides. Cytidine residues have been replaced by either 5-(1-propynyl) cytidine or phenoxazine and these positions are noted in Table 26. In combination, other residues have been replaced by uracil or 5-propynyl uracil and these are noted in the Table 26. All sequences have SEQ ID NO: 15.

TABLE 26

Phosphorothioate antisense oligonucleotides containing
modifications to the heterocycle

| | | | | | |
|---|---|---|---|---|---|
| | | 5-(1- | | | |
| | | propyn- | | 5- | |
| | NUCLEOTIDE SEQUENCE | yl | Phenox- | propynyl | |
| ISIS # | (5′→3′) | cytidine | azine | uracil | Uracil |

| | | | | | |
|---|---|---|---|---|---|
| 130599 | AGCTTCTTTGCACATGTAAA | 3,6, | none | 4,5,7,8, | None |
| | | 11,13 | | 9,15,17 | |
| 130719 | AGCTTCTTTGCACATGTAAA | None | 3,6,11, | 4,5,7,8, | none |
| | | | 13 | 9,15,17 | |
| 130724 | AGCTTCTTTGCACATGTAAA | none | 3,6,11, | none | 7,8,9 |
| | | | 13 | | |

### Example 27

Additional oligonucleotides designed to Human mdm2-Propynyl and phenoxazine modifications to the heterocycle

In accordance with the present invention, certain oligonucleotides were designed to target additional regions of the human mdm2 RNA, using published sequences (GenBank accession number Z12020, incorporated herein as SEQ ID NO: 1) with modifications to the heterocycle. The oligonucleotides are shown in Table 27. “Target site” indicates the first (5′-most) nucleotide number on the particular target sequence to which the oligonucleotide binds. All of the oligonucleotides are chimeric oligonucleotides (“gapmers”) 20 nucleotides in length, composed of a central “gap” region consisting of 2′-deoxynucleotides, which is flanked on both sides (5′ and 3′ directions) by nucleotide “wings”. The wings are composed of 2′-methoxyethyl (2′-MOE)nucleotides and are shown in bolded text. The internucleoside (backbone) linkages are phosphorothioate (P═S) throughout the oligonucleotides. All cytidine residues in ISIS 130600-130602 have been replaced by 5-(1-propynyl) cytidine while all cytidine residues in ISIS 130720-130722 and ISIS 130725-130727 have been replaced by phenoxazine. In combination, all thymidine residues in ISIS 130600-130602 and ISIS 130720-130722 have been replaced by 5-propynyl uracil while all thymidine residues in ISIS 130725-130727 have been replaced by uracil.

TABLE 27

Phosphorothioate antisense oligonucleotides con-
taining modifications to the sugar

| | | | | |
|---|---|---|---|---|
| | NUCLEOTIDE SEQUENCE | SEQ ID | | TARGET |
| ISIS # | (5′→3′) | NO | REGION | SITE |

| | | | | |
|---|---|---|---|---|
| 130600 | CAGGTTGTCTAAATTCCTAG | 212 | Coding | 1832 |
| 130601 | TGCCATGGACAATGCAACCA | 305 | Coding | 1652 |
| 130602 | GCTTATTCCTTTTCTTTAGC | 310 | Coding | 1712 |
| 130720 | CAGGTTGTCTAAATTCCTAG | 212 | Coding | 1832 |
| 130721 | TGCCATGGACAATGCAACCA | 305 | Coding | 1652 |
| 130722 | GCTTATTCCTTTTCTTTAGC | 310 | Coding | 1712 |
| 130725 | CAGGTTGTCTAAATTCCTAG | 212 | Coding | 1832 |
| 130726 | TGCCATCGACAATCCAACCA | 305 | Coding | 1652 |
| 130727 | GCTTATTCCTTTTCTTTAGC | 310 | Coding | 1712 |

### Example 28

Reduction of mdm2 mRNA levels in SJSA-1 cells by ISIS 16518

In accordance with the present invention, the reduction of mdm2 RNA levels was investigated in other cell types. SJSA-1 cells, an osteosarcoma cell line with increased mdm2 expression, were treated at 50, 100, 200 and 400 nm with ISIS 16518 and mRNA levels measured by Northern blot at endpoints of 6 and 24 hours post-treatment. Levels of p21 induction were also measured concurrently. The data are shown in Table 28.

TABLE 28
Mdm2 reduction and p21 induction in SJSA-1 cells after
treatment with ISIS 16518

% mRNA Inhibition

| Endpoint | 50 nM | 100 nM | 200 nM | 400 nM |
|---|---|---|---|---|
| mdm2 levels | 80 | 78 | 80 | 75 |
| (6 Hrs.) | | | | |
| mdm2 levels | 70 | 65 | 65 | 75 |
| (24 Hrs.) | | | | |

Fold Induction

| | | | | |
|---|---|---|---|---|
| p21 levels | 2.1 | 2.5 | 2.5 | 1.8 |
| (6 Hrs.) | | | | |
| P21 levels | 2.3 | 6.5 | 8 | 9 |
| (24 Hrs.) | | | | |

### Example 29

Effects of antisense inhibition of Human mdm2 expression on apoptosis

Using the flow cytometry technique of FACS (fluorescence-activated cell sorting) the induction of apoptosis, as a function of percent hypodiploidy, was measured in several cell lines after treatment with antisense oligonucleotides. HT1080 cells, a human fibrosarcoma cell line with low levels of mdm2 expression, were treated at doses of 50, 100, 200 and 300 nM with ISIS 16518, ISIS 116428, ISIS 111175, ISIS 119465 and the scrambled control, ISIS 17605 via the lipofectin mediated transfection protocol described previously. The levels of hypodiploidy of the treatment groups measured at 48 hours were compared to the control group which received no oligonucleotide treatment. No data is indicated by N.D. The data are shown in Table 29. The greatest amount of apoptosis is observed upon treatment with ISIS 119465 and ISIS 111175 and this occurred in a dose-dependent manner.

TABLE 29

Induction of apoptosis in HT1080 cells by antisense
oligonucleotides

| | | | |
|---|---|---|---|
| NUCLEOTIDE SEQUENCE | SEQ ID | TARGET | % Hypodiploidy |

| | | | | | | | |
|---|---|---|---|---|---|---|---|
| ISIS # | (5′→3′) | NO | SITE | 50 nM | 100 nM | 200 nM | 300 nM |

| | | | | | | | |
|---|---|---|---|---|---|---|---|
| — | No oligo group | — | — | N.D. | 1.6 | 1.7 | 1.6 |
| 17605 | Scrambled control | 24 | — | N.D. | 2.2 | 2.4 | 4.5 |
| 16518 | AGCTTCTTTGCACATGTAAA | 15 | 1695 | N.D. | 1.7 | 6.2 | N.D. |
| 116428 | TGCCATGGACAATGCAACCA | 305 | 1652 | N.D. | 4 | 5.5 | 9.8 |
| 111175 | AGCTTCTTTGCACATGTAAA | 15 | 1695 | 5 | 15 | 38 | N.D. |
| 119465 | AGCTTCTTTGCACATGTAAA | 15 | 1695 | 7 | 43 | 48 | N.D. |

In a similar experiment, SJSA-1 cells which have a high level of mdm2 expression were also treated with these oligonucleotides and apoptosis levels measured at 48 hours. These data are shown in Table 30. N.D. indicates no data for that treatment group. The data demonstrate that ISIS 111175 induces apoptosis to the greatest extent and that this increase occurs in a dose-dependent manner.

TABLE 30

Induction of apoptosis in SJSA-1 cells by antisense
oligonucleotides

| | | | | | | |
|---|---|---|---|---|---|---|
| | | | | % | | |
| | NUCLEOTIDE SEQUENCE | | TARGET | Hypodiploidy | | |
| ISIS # | (5′→3′) | SEQ ID NO | SITE | 100 nM | 200 nM | 300 nM |

| | | | | | | |
|---|---|---|---|---|---|---|
| — | No oligo group | — | — | 3.8 | N.D. | N.D. |
| 17605 | Scrambled control | 24 | — | .5 | 1.5 | 7 |
| 16518 | AGCTTCTTTGCACATGTAAA | 15 | 1695 | 1.0 | 3.5 | N.D. |
| 116428 | TGCCATGGACAATGCAACCA | 305 | 1652 | 2.1 | 4.1 | 10.1 |
| 111175 | AGCTTCTTTGCACATGTAAA | 15 | 1695 | 17 | 35 | 45 |

### Example 30

Effects of antisense inhibition of Human mdm2 expression on apoptosis-A549 cells

In a similar experiment, human A549 cells were treated with 200 nM of antisense oligonucleotides and levels of apoptosis were measured at 24 and 48 hours. The data are shown in Table 31. N.D. indicates no data. The data demonstrate that ISIS 111173 and ISIS 119465 each induce apoptosis in a time-dependent manner and to the greatest extent.

TABLE 31

Induction of apoptosis in A549 cells by antisense
oligonucleotides

| | | | | | |
|---|---|---|---|---|---|
| | | | | % | % |
| | NUCLEOTIDE SEQUENCE | SEQ ID | TARGET | Hypodiploidy (24 | Hypodiploidy (48 |
| ISIS # | (5′→3′) | NO | SITE | Hr.) | Hr.) |

| | | | | | |
|---|---|---|---|---|---|
| 17605 | Scrambled control | 24 | — | 1.5 | 0.8 |
| 16518 | AGCTTCTTTGCACATGTAAA | 15 | 1695 | 3.2 | 3.1 |
| 105271 | AGCTTCTTTGCACATGTAAA | 15 | 1695 | 1.8 | 3.6 |
| 116428 | TGCCATGGACAATGCAACCA | 305 | 1652 | 5.4 | 7.1 |
| 116433 | GCTTATTCCTTTTCTTTAGC | 310 | 1712 | 2.0 | 4.6 |
| 31539 | CAGGTTGTCTAAATTCCTAG | 212 | 1832 | 1.7 | 1.5 |
| 111173 | AGCTTCTTTGCACATGTAAA | 15 | 1695 | 8 | 28 |
| 119465 | AGCTTCTTTGCACATGTAAA | 15 | 1685 | 10 | 35 |

### Example 31

Effects of antisense inhibition of Human mdm2 expression on apoptosis-HeLa cells

To investigate the effects of p53 status (p53 is a tumor suppressor gene) on the effects of the antisense oligonucleotides, HeLa cells, which have a mutant p53, were treated with ISIS 16518, ISIS 116428 and the scrambled control, ISIS 17605 at 100 and 200 nM and FACS analysis was performed at 24 and 48 hours post-treatment. The data are shown in Table 32. It was determined that ISIS 16518 and ISIS 116428 have different affects on apoptosis in HeLa cells.

TABLE 32

Induction of apoptosis in HeLa cells by antisense
oligonucleotides

| | | | |
|---|---|---|---|
| NUCLEOTIDE SEQUENCE | TARGET | 24 HOURS | 48 HOURS |

| | | | | | | | |
|---|---|---|---|---|---|---|---|
| ISIS # | (5′→3′) | SEQ ID NO | SITE | 100 nM | 200 nM | 100 nM | 200 nM |

| | | | | | | | |
|---|---|---|---|---|---|---|---|
| 17605 | Scrambled control | 24 | — | 2.5 | 3 | 3 | 3 |
| 16518 | AGCTTCTTTGCACATGTAAA | 15 | 1695 | 6.5 | 15 | 15 | 22 |
| 116428 | TGCCATGGACAATGCAACCA | 305 | 1652 | 3.5 | 5.5 | 6 | 7.5 |

### Example 32

Inhibition of mdm2 and induction of apoptosis by a series of modified antisense oligonucleotides-16518 series

Derivatives of ISIS 16518 (SEQ ID NO: 15), a chimeric oligonucleotide described previously, were investigated for improved properties of target reduction and induction of apoptosis in HT1080, SJSA-1 and A549 cells.

Cells were treated with ISIS 130599 (propyne derivative), ISIS 130724 (phenoxazine derivative) and ISIS 130719 (propyne/phenoxaxine derivative) at doses of 50, 100 and 300 nM for Northern blot analysis of mdm2 mRNA expression. Results were compared to ISIS 16518.

For FACS analyses, cells were treated with 100, 200 and 300 nM doses and percent hypodiploidy (measure of apoptosis) compared to that of ISIS 16518. The data are shown in table 33. N.D. indicates no data.

TABLE 33
Reduction of mdm2 expression and induction of apoptosis in
cells by modified antisense oligonucleotides

| | mdm2 target expression (% Inhibition) | | | | | | | | |
|---|---|---|---|---|---|---|---|---|---|
| | HT1080 | | | SJSA-1 cells | | | A549 cells | | |
| ISIS # | 50 nM | 100 nM | 300 nM | 50 nM | 100 nM | 300 nM | 50 nM | 100 nM | 300 nM |
| 16518 | 0 | 20 | 80 | 50 | 60 | 40 | 50 | 75 | 75 |
| 130599 | 0 | 80 | 96 | 25 | 40 | 70 | 50 | 80 | 95 |
| 130724 | 0 | 40 | 70 | N.D. | N.D. | N.D. | N.D. | N.D. | N.D. |
| 130719 | 0 | 75 | 98 | N.D. | N.D. | N.D. | N.D. | N.D. | N.D. |
| | Induction of Apoptosis (% Hypodiploidy) | | | | | | | | |
| | HT1080 cells | | | SJSA-1 cells | | | A549 cells | | |
| | 100 nM | 200 nM | 300 nM | 100 nM | 200 nM | 300 nM | 100 nM | 200 nM | 300 nM |
| 16518 | N.D. | N.D. | N.D. | 3 | 6 | 8 | 3 | 5 | 24 |
| 130599 | N.D. | N.D. | N.D. | 7 | 9 | 14 | 18 | 30 | 38 |
| 130724 | N.D. | N.D. | N.D. | 1.5 | 2.5 | 4.5 | N.D. | N.D. | N.D. |
| 130719 | N.D. | N.D. | N.D. | N.D. | N.D. | N.D. | N.D. | N.D. | N.D. |

### Example 33

Inhibition of mdm2 and induction of apoptosis by a series of modified antisense oligonucleotides-116428 series

Derivatives of ISIS 116428 (SEQ ID NO: 305), a chimeric oligonucleotide described previously, were investigated for improved properties of mdm2 mRNA target reduction and induction of apoptosis in HT1080, SJSA-1 and A549 cells.

Cells were treated with ISIS 130601 (propyne derivative), ISIS 130726 (phenoxazine derivative) and ISIS 130721 (propyne/phenoxaxine derivative) at doses of 50, 100 and 300 nM for Northern blot analysis of mdm2 mRNA expression. Results were compared to ISIS 116428.

For FACS analyses, cells were treated with 100, 200 and 300 nM doses and percent hypodiploidy (measure of apoptosis) compared to that of ISIS 116428. The data are shown in Table 34.

TABLE 34
Reduction of mdm2 expression and induction of apoptosis in
cells by modified antisense oligonucleotides

| | mdm2 target expression (% Inhibition) | | | | | | | | |
|---|---|---|---|---|---|---|---|---|---|
| | HT1080 | | | SJSA-1 cells | | | A549 cells | | |
| ISIS # | 50 nM | 100 nM | 300 nM | 50 nM | 100 nM | 300 nM | 50 nM | 100 nM | 300 nM |
| 116428 | 0 | 0 | 99 | 0 | 75 | 75 | 20 | 50 | 75 |
| 130601 | 0 | 75 | 95 | 0 | 75 | 75 | 40 | 50 | 70 |
| 130726 | 0 | 80 | 95 | N.D. | N.D. | N.D. | N.D. | N.D. | N.D. |
| 130721 | 0 | 75 | 98 | N.D. | N.D. | N.D. | N.D. | N.D. | N.D. |
| | Induction of Apoptosis (% Hypodiploidy) | | | | | | | | |
| | HT1080 cells | | | SJSA-1 cells | | | A549 cells | | |
| | 100 nM | 200 nM | 300 nM | 100 nM | 200 nM | 300 nM | 100 nM | 200 nM | 300 nM |
| 116428 | N.D. | N.D. | N.D. | 3 | 7 | 9 | 3 | 10 | 12 |
| 130601 | N.D. | N.D. | N.D. | 10 | 8 | 25 | 5 | 32 | 37 |
| 130726 | N.D. | N.D. | N.D. | 1.5 | 5.8 | 11 | N.D. | N.D. | N.D. |
| 130721 | N.D. | N.D. | N.D. | N.D. | N.D. | N.D. | N.D. | N.D. | N.D. |

### Example 34

Use of CYTOFECTIN™ reagent to improve in vitro delivery of antisense oligonucleotides in SJSA-1 cells

In accordance with the present invention, the antisense oligonucleotide delivery properties of the transfection reagent, Cytofectin™, were investigated.

In these studies, SJSA-1 cells were treated with a series of derivatives of the chimeric phosphorothioate oligonucleotide, ISIS 16518 (SEQ ID NO 15). ISIS 111175 (contains one G-clamp) and ISIS 119465 (contains two G-clamps) each contain at least one G-clamp, while ISIS 130599 is a propyne derivative. ISIS 130599 contains 5-propynyl cytidine at positions 3, 6, 11 and 13 in addition to 5-propynyluracil at positions 4, 5, 7,8 9, 15 and 17. The control olignonucleotide, ISIS 133541 (TTCGACAGATCTCTATAGTA; SEQ ID NO 319) contains one G-clamp at position 6 and is a scramble of ISIS 16518.

Doses were 0.5, 1, 5, 10, 50 and 100 nM for four hours in the presence of 6 g/ML CYTOFECTIN™, washed and allowed to recover for an additional 20 hours. Total RNA was extracted and 15-20 g of each was resolved on 1% gels and transferred to nylon membranes. The blots were probed with a ³²p radiolabeled mdm2 cDNA probe and then stripped and reprobed with a radiolabeled G3PDH probe to confirm equal RNA loading. Levels of mdm2 and p21 transcripts were examined and quantified with a PhosphorImager (Molecular Dynamics, Sunnyvale, Calif.). Results are shown in Table 35.

In this experiment, levels of mdm2 expression are reduced upon treatment with all oligonucleotides relative to control with the greatest reduction occurring upon treatment with the G-clamp antisense oligonucleotides. At the same time, there was a six fold induction of p21 levels in the G-clamp treatment group as compared to a four-fold induction in the ISIS 16l58 treated group relative to control. Comparisons with the propyne derivative reveal the same trends with a decrease in mdm2 expression level and and increase in p21 levels. Cytofectin™ therefore, can be used as an effective transfection reagent with antisense oligonucleotides containing a variety of chemical modifications. In addition, it is clear that the G-clamp oligonucleotides are most effective in reducing mdm2 expression levels in this assay.

TABLE 35
Reduction of mdm2 expression levels in SJSA-1 cells by
antisense oligonucleotides transfected with Cytofectin ™

| | % Reduction mdm2 | | | | | | Fold Induction p21 | | | | | |
|---|---|---|---|---|---|---|---|---|---|---|---|---|
| | Oligonucleotide Dose (nM) | | | | | | Oligonucleotide Dose (nM) | | | | | |
| Isis # | 0.5 | 1 | 5 | 10 | 50 | 100 | 0.5 | 1 | 5 | 10 | 50 | 100 |
| 16518 | 15 | 25 | 40 | 60 | 65 | 70 | 1.5 | 2 | 3.5 | 4 | 4 | 4 |
| 111175 | 70 | 60 | 75 | 75 | 85 | 90 | 1.5 | 2.5 | 5 | 5.5 | 6 | 5.5 |
| 133541 | 40 | 45 | 50 | 45 | 30 | 20 | 1 | 1 | 1 | 1 | 1 | 1 |
| 119465 | 50 | 60 | 70 | 80 | 90 | 85 | 1.8 | 2.4 | 3.8 | 4.5 | 5.5 | 5.5 |
| 130599 | 60 | 75 | 80 | 70 | 75 | 75 | 1.5 | 1.7 | 3.3 | 3.5 | 3.5 | 2.5 |

In a similar experiment using the same transfection protocol, SJSA-1 cells were treated with a series of propynyl derivatives of the chimeric phosphorothioate oligonucleotides, ISIS 16518 (SEQ ID NO 15), ISIS 31539 (SEQ ID NO 212) and ISIS 116428 (SEQ ID NO 305).

ISIS 130599 described previously and its mismatch control ISIS 138222 (SEQ ID NO 320; AAATGTACACGTTTCTTCGA; containing 5-propynyluracil at positions 4, 6, 12, 13, 14, 16 and 17 and 5-(1-propynyl)cytidine at positions 8, 10 and 18) are propyne derivatives of ISIS 16518.

ISIS 130600 described previously and its mismatch control ISIS 138223 (SEQ ID NO 321; GATCCTTAAATCTGTTGGAC; containing 5-propynyluracil at positions 3, 6, 7, 11, 13, 15 and 16 and 5-(l-propynyl)cytidine at positions 4, 5, 12 and 20) are propyne derivatives of ISIS 31539.

ISIS 130601 described previously and its mismatch control ISIS 138224 (SEQ ID NO 322; ACCAACGTAACAGGTACCGT; containing 5-propynyluracil at positions 8, 15 and 20 and 5-(l-propynyl)cytidine at positions 2, 3, 6, 11, 17 and 18 are propyne derivatives of ISIS 116428.

Doses were 0.1, 0.5, 5, 10 and 100 nM for four hours in the presence of 6 μg/mL CYTOFECTIN™, washed and allowed to recover for an additional 20 hours. Total RNA was extracted and 15-20 μg of each was resolved on 1% gels and transferred to nylon membranes. The blots were probed with a ³²p radiolabeled mdm2 cDNA probe and then stripped and reprobed with a radiolabeled G3PDH probe to confirm equal RNA loading. Levels of mdm2 and p21 transcripts were examined and quantified with a PhosphorImager (Molecular Dynamics, Sunnyvale, Calif.). Results are shown in Table 36.

In this experiment, levels of mdm2 expression are reduced upon treatment with all oligonucleotides relative to control with the greatest reduction occurring upon treatment with the propynyl antisense oligonucleotides. At 5 the same time, there was a five-fold induction of p21 levels in the propynyl treatment group relative to control. Comparisons with the G-clamp derivative reveals the same trends with a decrease in mdm2 expression level and and increase in p21 levels.

TABLE 36
Reduction of mdm2 expression levels in SJSA-1 cells by
propynyl antisense oligonucleotides transfected with
Cytofectin ™

| | % Reduction of mdm2 | | | | | Fold Induction p21 | | | | |
|---|---|---|---|---|---|---|---|---|---|---|
| | Oligonucleotide Dose (nM) | | | | | Oligonucleotide Dose (nM) | | | | |
| Isis # | 0.1 | 0.5 | 5 | 10 | 100 | 0.1 | 0.5 | 5 | 10 | 100 |
| 16518 | 15 | 17 | 22 | 62 | 65 | 1 | 1.1 | 1.5 | 2.5 | 2.3 |
| 130599 | 25 | 52 | 68 | 62 | 65 | 1 | 1.2 | 2.3 | 3 | 2.4 |
| (propyne) | | | | | | | | | | |
| 138222 | 10 | 12 | 10 | 18 | 20 | 1 | 1 | 1 | 1.3 | 2 |
| (control) | | | | | | | | | | |
| 31539 | 0 | 0 | 0 | 18 | 50 | 1 | 1.2 | 1.7 | 2.5 | 2.8 |
| 130600 | 0 | 0 | 18 | 50 | 65 | 1.1 | 1.2 | 1.8 | 3.2 | 3.4 |
| (propyne) | | | | | | | | | | |
| 138223 | 0 | 18 | 0 | 0 | 22 | 1 | 1 | 1 | 1.1 | 1.3 |
| (control) | | | | | | | | | | |
| 116428 | 15 | 5 | 10 | 42 | 60 | 1 | 1 | 1.3 | 2.4 | 3.5 |
| 130601 | 15 | 42 | 53 | 53 | 60 | 1.1 | 1.3 | 1.7 | 3.3 | 5 |
| (propyne) | | | | | | | | | | |
| 138224 | 10 | 0 | 0 | 0 | 0 | 1 | 1 | 1 | 1.1 | 1.3 |
| (control) | | | | | | | | | | |

### Example 35

Time course studies of the effects of antisense inhibition of mdm2 expression in SJSA-1 cells by G-clamp antisense oligonucleotides

In accordance with the present invention, time-course studies were performed to compare the reduction in mdm2 expression levels by antisense oligonucleotides containing various chemistries.

In these studies, SJSA-1 cells were treated with 100 and 200 nM of a series of derivatives of the chimeric phosphorothioate oligonucleotide, ISIS 16518 (SEQ ID NO 15). Antisense oligonucleotides previously described and containing two G-clamp modifications (ISIS 111173, 111176 and 119465) were compared to ISIS 16518 and 116428 for their ability to reduce mdm2 expression over time. The control, ISIS 133543 (TTCGACAGATCTCTATAGTA, SEQ ID NO 323; contains a G-clamp in positions 3 and 13), was a chimeric oligonucleotide (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide.

At time points of 6, 24 and 48 hours after treatment, total RNA was extracted and 15-20 μg of each was resolved on 1% gels and transferred to nylon membranes. The blots were probed with a ³²p radiolabeled mdm2 cDNA probe and then stripped and reprobed with a radiolabeled G3PDH probe to confirm equal RNA loading. Levels of mdm2 transcripts were examined and quantified with a PhosphorImager (Molecular Dynamics, Sunnyvale, Calif.). Results are shown in Table 37. From the data, ISIS 111173 has the greatest reduction of target expression and the longest duration of action. In general, the G-clamp containing oligonucleotides showed the greatest reduction in expression as well as the longest duration of action.

TABLE 37
Effects of G-clamp antisense oligonucleotides on mdm2
expression over time

% Reduction mdm2

| | 6 Hr. | 6 Hr. | 24 Hr. | 48 Hr. |
|---|---|---|---|---|
| ISIS # | (100 nM) | (200 nM) | (100 nM) | (100 nM) |
| Saline | 0 | 0 | 0 | 0 |
| 133543 | 70 | 18 | 10 | 0 |
| (control) | | | | |
| 111173 | 98 | 95 | 99 | 95 |
| 111178 | 90 | 98 | 93 | 85 |
| 119465 | 94 | 85 | 85 | 79 |
| 16518 | 90 | 70 | 85 | 70 |
| 116428 | 82 | 85 | 70 | 10 |

### Example 36

Antisense oligonucleotides designed to mouse mdm2.

In accordance with the present invention, oligonucleotides were designed to target regions of the mouse mdm2 RNA, using published sequences (GenBank accession number U47934, incorporated herein as SEQ ID NO: 324). The oligonucleotides are shown in Table 38. “Target site” indicates the first (5′-most) nucleotide number on the particular target sequence to which the oligonucleotide binds. All compounds in Table 38 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines.

TABLE 38

Nucleotide Sequence of Mouse mdm2 chimeric phos-
phorothioate oligonucleotides having 2′-MOE wings
and a deoxy gap

| | | | | |
|---|---|---|---|---|
| | NUCLEOTIDE SEQUENCE | SEQ ID | | TARGET |
| ISIS # | (5′→3′) | NO | REGION | SITE |

| | | | | |
|---|---|---|---|---|
| 27172 | GGTAGACACAGACATGTTGG | 325 | Coding | 11 |
| 27173 | TGGTCTAACCAGAGTCTCTT | 326 | Coding | 71 |
| 27174 | TCACAGAGAAACTCGGGACT | 327 | Coding | 261 |
| 27175 | AGATCATTGCATATATTTTC | 328 | Coding | 291 |
| 27176 | QTGCCAGAGTCTTGCTGACT | 329 | Coding | 331 |
| 27177 | ACTCCCACCTTCAGGCTGAC | 330 | Coding | 371 |
| 11649 | GATCACTCCCACCTTCAGGC | 331 | Coding | 375 |
| 27178 | GAAGATGPAGGTTTCTCTTC | 332 | Coding | 421 |
| 27179 | GATGAGGTAGACAGTCTAGA | 333 | Coding | 451 |
| 27180 | TCTTCTGTCTCACTAATGGA | 334 | Coding | 481 |
| 27181 | CAGGTAGCTCATCTGTGTTC | 335 | Coding | 501 |
| 27182 | GCGCTTCCGGTGCCGCTCCC | 336 | Coding | 521 |
| 27183 | TCAAAGGACAGGGACCTGCG | 337 | Coding | 541 |
| 27184 | CACACAGACCCAGGCTCGGA | 338 | Coding | 561 |
| 27185 | TGCTGCCGCCGCTGCACATC | 339 | Coding | 591 |
| 27186 | TGGACTCGCTGCTGCTGCTG | 340 | Coding | 621 |
| 27187 | CTTACGCCATCGTCAAGATC | 341 | Coding | 661 |
| 27188 | AGAAACTGAATCCTGATCCA | 342 | Coding | 701 |
| 27189 | AGTCCAGAGACTCAACTTCA | 343 | Coding | 741 |
| 27190 | GTGACCCGATAGACCTCATC | 344 | Coding | 811 |
| 27191 | TCTGTATCGCTTTCTCCTGT | 345 | Coding | 841 |
| 27192 | GCATCTTTTGCAGTGTGATG | 346 | Coding | 941 |
| 27193 | GTCTGCAAGCCAGTTCTCAC | 347 | Coding | 971 |
| 27194 | TGGCTTTTTCAGAGATTTCC | 348 | Coding | 1011 |
| 27195 | TGGCTGCTATAAACAATGCT | 349 | Coding | 1201 |
| 27196 | CTAGATTCCACACTCTCGTC | 350 | Coding | 1261 |
| 27197 | CAGCCATTTTTAGGCCGCCC | 351 | Coding | 1321 |
| 105789 | AGCTTCTTTGCACACGTGAA | 352 | Coding | 1378 |
| 27198 | TTTAGCTTCTTTGCACACGT | 353 | Coding | 1381 |
| 27199 | CTGCACACTGGGCAGGGCTT | 354 | Coding | 1411 |
| 27200 | TAAGTTAGCACAATCATTTG | 355 | Coding | 1441 |

### Example 37

Additional antisense oligonucleotides designed to nucleotides 1261-1280 of mouse mdm2-Modifications to the heterocycle

In accordance with the present invention, a series of oligonucleotides having the starting sequence of ISIS 27196 were designed to incorporate the G-clamp modification described previously. These oligonucleotides are shown in Table 39. The oligonucleotides are phosphorothioate oligonucleotides 20 nucleotides in length composed of a ten 2′-deoxynucleotide central “gap” region flanked on both sides (5′ and 3′ directions) by five-nucleotide “wings”. The wings are composed of 2′-methoxyethyl(2′-MOE)nucleotides. All other nucleotides are 2′deoxyribose throughout the oligonucleotide. The internucleoside (backbone) linkages are phosphorothioate throughout the oligonucleotides. As noted in Table 39 in bolded notation, certain cytosines have been replaced with the cytosine derivative, 1,3-diazaphenoxazine-2-one (G-clamp). All other cytidine residues are 5-methylcytidines. All sequences have SEQ ID NO: 15.

TABLE 39

Additional antisense oligonucleotides targeting
mouse mdm2 containing G-clamp modifications

| | |
|---|---|
| | NUCLEOTIDE SEQUENCE |
| ISIS # | (5′→3′) |

| | |
|---|---|
| 143704 | CTAGATTCCACACTCTCGTC |
| 143705 | CTAGATTCCACACTCTCGTC |
| 143706 | CTAGATTCCACACTCTCGTC |
| 143707 | CTAGATTCCACACTCTCGTC |
| 143708 | CTAGATTCCACACTCTCGTC |
| 143709 | CTAGATTCCACACTCTCGTC |
| 143710 | CTAGATTCCACACTCTCGTC |

### Example 38

Oligonucleotides designed to nucleotides 2161-1280 of mouse mdm2-Propynyl and phenoxazine modifications to the heterocycle

In accordance with the present invention, a series of oligonucleotides having the starting sequence of ISIS 27196 were designed to incorporate the propynyl and phenoxazine modifications described previously. The oligonucleotides are shown in Table 40. All of the oligonucleotides are chimeric oligonucleotides (“gapmers”) 20 nucleotides in length, composed of a central “gap” region consisting of 2′-deoxynucleotides, which is flanked on both sides (5′ and 3′ directions) by nucleotide “wings”. The wings are composed of 2′-methoxyethyl (2′-MOE)nucleotides and are shown in bolded text. The internucleoside (backbone) linkages are phosphorothioate (P═S) throughout the oligonucleotides. Cytidine residues have been replaced by either 5-(1-propynyl) cytidine or phenoxazine and these positions are noted in Table 40. In combination, other residues have been replaced by uracil or 5-propynyl uracil and these are also noted in the Table 40. All sequences have SEQ ID NO: 15.

TABLE 40

Phosphorothioate antisense oligonucleotides containing
propyne and phenoxazine modifications to the heterocycle

| | | | | | |
|---|---|---|---|---|---|
| | NUCLEOTIDE SEQUENCE | 5-(1-propynyl) | Phen- | 5-propynyl | |
| ISIS # | (5′→3′) | cytidine | oxazine | uracil | Uracil |

| | | | | | |
|---|---|---|---|---|---|
| 13063 | CTAGATTCCACACTCTCGTC | 1, 8, 9, | None | 2, 6, | None |
| | | 11, 13, | | 7, 14, | |
| | | 15, 17, | | 16, 19 | |
| | | 20 | | | |
| 130723 | CTAGATTCCACACTCTCGTC | None | 1, 8, 9, | 2, 6, | None |
| | | | 11, 13, | 7, 14, | |
| | | | 15, 17, | 16, 19 | |
| | | | 20 | | |
| 130728 | CTAGATTCCACACTCTCGTC | None | 1, 8, 9, | None | 6, 7, |
| | | | 11, 13, | | 14 |
| | | | 15, 17, | | |
| | | | 20 | | |

### Example 39

Effects of cellular p53 status on the activity of antisense oligonucleotides targeting mdm2 in vitro

It is known that, in addition to mediating p53 degradation, the mdm2 promoter contains a p53 response element. It is therefore likely that p53 participates in a feedback loop that regulates the expression of mdm2.

In an effort to elucidate the underlying mechanism of this feedback loop, species-specific antisense oligonucleotides designed to human mdm2 (ISIS 16518; SEQ ID NO: 15) and mouse mdm2 (ISIS 27196; SEQ ID NO: 350) were tested in both in vitro and in vivo experiments for their reduction of mdm2 levels and induction of p21 levels.

HCT116 cells and a derivative thereof (containing a disruption in the p53 gene (p53 −/−) generated by the methods of Bunz, F., et al., Science, 1998, 282, 1497-1501) are human colorectal carcinoma cells.

HCT116 and HCT116 (p53 −/−) cells were routinely cultured in complete McCoy's 5A basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells were routinely passaged by trypsinization and dilution when they reached 90% confluence.

Wild-type HCT116 (p53 +/+) and HCT116 cells homozygous for the absence of p53 (p53 −/−) were treated with 50, 100, 200 and 300 nM ISIS 16518, ISIS 116428, ISIS 111173, ISIS 119465 and ISIS 111178 and levels of mdm2 and p21 RNA were measured at 6 hours post-treatment.

It was found that for all antisense oligonucleotides tested, mdm2 levels were reduced in both wild-type and (p53 −/−) but reduced more efficiently in HCT116 (p53 −/−) cells. ISIS 111173 was found to be the most potent oligonucleotide in reducing mdm2 levels. The kinetics of mdm2 expression recovery was found to coincide with the induction of p21 expression in wild-type but not (p53 −/−) cells. Wild-type HCT116 cells were also shown to express p21 at a level three times that of the (p53 −/−) cells. The fact that mdm2 antisense oligonucleotide treatment in the deletion mutant (p53−/−) resulted in sustained reduction of mdm2 expression with no induction of p21 indicates that an autoregulatory feedback loop involving p53 and mdm2 does exist and explains the inefficient nature of antisense reduction of mdm2 in wild-type cells. It was also determined that mdm2 RNA levels in HCT116 (p53 −/−) cells decreases to half of control levels by 72 hours after plating as the cells become more confluent, further supporting the necessity of p53 to maintain constant mdm2 levels.

In a similar experiment, wild-type (p53 +/+) and HCT116 cells homozygous for the absence of p53 (p53 −/−) were treated with 50, 100 and 200 nM ISIS 16518, ISIS 116428, ISIS 111173, ISIS 119465 and ISIS 111178 and levels of apoptosis were measured at 24 and 48 hours after treatment. It was found that (p53−/−) cells were more sensitive to antisense oligonucleotide-induced apoptosis by a factor of 3 than wild-type cells suggesting that induction of apoptosis by mdm2 antisense oligonucleotides is p53 independent.

### Example 40

Effects of cellular p53 status on the activity of antisense oligonucleotides targeting mdm2 in vivo

Using the species-specific antisense oligonucleotide designed to mouse mdm2 (ISIS 27196; SEQ ID NO: 350), mice either homozygous (p53 −/−) or heterozygous (p53 −/−) for a deletion in p53 as well as wild type mice (p53 +/+) were treated with saline or antisense oligonucleotide and levels of mdm2 and p21 were measured by RPA. All mice were treated at a dose of 25 mg/kg of ISIS 27196 twice daily for 8 days after which the animals were sacrificed and livers isolated for RPA analysis as described in other examples herein. RPA blots were quantified with a PhosphorImager (Molecular Dynamics, Sunnyvale, Calif.) and are averages of three replicates. Data are expressed in arbitrary units and detected levels of mdm2 and p21 have been normalized to the level of G3PDH. The data are shown in Table 41.

TABLE 41
RPA Evaluation of p53 knockout mice
treated with ISIS 27196

| | |
|---|---|
| Saline | Oligonucleotide Treatment |

| | Mdm2 | p21 | Mdm2 | p21 |
|---|---|---|---|---|
| p53 −/− | .99 | .12 | .47 | .08 |
| p53 −/+ | .99 | .13 | .84 | .85 |
| p53 +/+ | 1.14 | .34 | .81 | .72 |

Mdm2 antisense oligonucleotide treatment had a 50% reduction in mdm2 RNA (p=001) in (p53 −/−) mice and no effect on mdm2 expression in heterozygous or wild-type mice. No induction of p21 RNA was observed in (p53 −/−) mice, while mice heterozygous for p53 showed a 9-fold induction of p21 RNA (p=0.0004). Wild-type mice had a 2.3-fold induction of p21 RNA (p=0.02) and were observed to have a 3 fold higher level of basal expression of p21 than heterozygous mice (p=0.2) or homozygous mice (p=0.16).

### Example 41

Antisense oligonucleotides designed to target a variant of the 5′ UTR of human mdm2

In accordance with the present invention, oligonucleotides were designed to target a variant of the 5′ untranslated region of Human mdm2 RNA, using published sequences (GenBank accession number U28935, incorporated herein as SEQ ID NO: 2). The oligonucleotides are shown in Table 2. “Target site” indicates the first (5′-most) nucleotide number on the particular target sequence to which the oligonucleotide binds. All compounds in Table 15 are chimeric oligonucleotides (“gapmers”) 20 nucleotides in length, composed of a central “gap” region consisting of ten 2′-deoxynucleotides, which is flanked on both sides (5′ and 3′directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine resides are 5-methylcytidines.

TABLE 42

Chimeric phosphorothioate antisense oligonucleo-
tides designed to target a variant of the 5′ un-
translated region of Human mdm2

| | | | | |
|---|---|---|---|---|
| | | SEQ | | TAR- |
| | NUCLEOTIDE SEQUENCE | ID | | GET |
| ISIS # | (5′→3′) | NO | REGION | SITE |

| | | | | |
|---|---|---|---|---|
| 107973 | CTGAACACAGCTGGGAAAAT | 356 | Junction | 221 |
| | | Intron: Exon | | |
| 107974 | CGCCACTGAACACAGCTGGG | 357 | Junction | 226 |
| | | | Intron: Exon | |
| 107975 | ATCGCCACTGAACACAGCTG | 358 | Junction | 228 |
| | | | Intron: Exon | |
| 107976 | TCCAATCGCCACTGAACACA | 359 | Exon 2 | 232 |
| 107977 | CCTCCAATCGCCACTGAACA | 360 | Exon 2 | 234 |
| 107978 | ACCCTCCAATCGCCACTGAA | 361 | Exon 2 | 236 |
| 107979 | CAGGTCTACCCTCCAATCGC | 362 | Exon 2 | 243 |
| 107980 | CCACAGGTCTACCCTCCAAT | 363 | Exon 2 | 246 |

### Example 42

Additional oligonucleotides targeting a variant of the 5′ UTR of human mdm2- MOE modification throughout

In a further embodiment, additional antisense oligonucleotides were designed to incorporate the 2′-methoxyethyl (2′-MOE) chemistry throughout the oligonucleotide. These oligonucleotides are shown in Table 43. “Target site” indicates the first (5′-most) nucleotide number on the particular target sequence to which the oligonucleotide binds. All compounds in Table 43 are 20 nucleotides in length, composed of 2′-methoxyethyl (2′-MOE)nucleotides. The internucleoside (backbone) linkages are phosphorothioate (P═S) throughout the oligonucleotide. All cytidine residues are 5-methylcytidines.

TABLE 43

Phosphorothioate antisense oligonucleotides de-
signed to target a variant of the 5′ untranslated
region of Human mdm2

| | | | | |
|---|---|---|---|---|
| | | SEQ | | TAR- |
| | NUCLEOTIDE SEQUENCE | | | GET |
| ISIS # | (5′→3′) | ID NO | REGION | SITE |

| | | | | |
|---|---|---|---|---|
| 108486 | CTGAACACAGCTGGGAAAAT | 356 | Intron:Exon | 221 |
| | | | Junction | |
| 108487 | CGCCACTGAACACAGCTGGG | 357 | Intron:Exon | 226 |
| | | | Junction | |
| 108488 | ATCGCCACTGAACACAGCTG | 358 | Intron: Exon | 228 |
| | | | Junction | |
| 108489 | TCCAATCGCCACTGAACACA | 359 | Exon 2 | 232 |
| 108490 | CCTCCAATCGCCACTGAACA | 360 | Exon 2 | 234 |
| 108491 | ACCCTCCAATCGCCACTGAA | 361 | Exon 2 | 236 |
| 108492 | CAGGTCTACCCTCCAATCGC | 362 | Exon 2 | 243 |
| 108493 | CCACAGGTCTACCCTCCAAT | 363 | Exon 2 | 246 |
| 107981 | AAAAGACACGATGAAAACTG | 364 | Intron 2 | 391 |
| 107982 | GAAAAAAAAGACACGATGAA | 365 | Intron 2 | 396 |
| 107983 | ACAAGGAAAAAAAAGACACG | 366 | Intron 2 | 401 |
| 107984 | TGCCTACAAGGAAAAAAAAG | 367 | Intron 2 | 406 |
| 107985 | ACATTTGCCTACAAGGAAAA | 368 | Intron 2 | 411 |
| 107986 | ATTGCACATTTGCCTACAAG | 369 | Intron 2 | 416 |

### Example 43

Antisense oligonucleotides designed to nucleotides 241-260 and 238-257 of a variant of the 5′ UTR of human mdm2

In a further embodiment, additional antisense oligonucleotides, were designed to target the 5′ UTR variant beginning at nucleotide 241 or 238. The oligonucleotides are shown in Table 44. All compounds in Table 44, are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines.

TABLE 44

Chimeric phosphorothicate antisense oligonucleo-
tides designed to target nucleotides 238-257 and
241-260 of a variant of the 5′ untranslated region
of Human mdm2

| | | | | |
|---|---|---|---|---|
| | NUCLEOTIDE SEQUENCE | SEQ ID | | TARGET |
| ISIS # | (5′→3′) | NO | REGION | SITE |

| | | | | |
|---|---|---|---|---|
| 107990 | CTACCCTCCAATCGCCACTG | 28 | Exon 2 | 238 |
| 107991 | CTACCCTCCAATCGCCACTG | 28 | Exon 2 | 238 |
| 107992 | GGTCTACCCTCCAATCGCCA | 29 | Exon 2 | 241 |
| 107993 | GGTCTACCCTCCAATCGCCA | 29 | Exon 2 | 241 |
| 108484 | CTACCCTCCAATCGCCACTG | 28 | Exon 2 | 238 |
| 108485 | GGTCTACCCTCCAATCGCCA | 29 | Exon 2 | 241 |

### Example 44

Effects of antisense oligonucleotides designed to target genomic regions of human mdm2 on the expression of mdm2

In accordance with the present invention, additional oligonucleotides were designed to target genomic regions of the human mdm2 RNA, using published sequences (GenBank accession number U39736, incorporated herein as SEQ ID NO: 370). The oligonucleotides are shown in Table 45. “Target site” indicates the first (5′-most) nucleotide number on the particular target sequence to which the oligonucleotide binds. All compounds in Table 45 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines.

TABLE 45

Inhibiton of Hunian mdm2 mRNA expression by chimeric
phosphorothioate oligonucleotides designed to genomic
regions of the Hunian mdm2 gene

| | NUCLEOTIDE SEQUENCE | | | | |
|---|---|---|---|---|---|
| ISIS # | (5′→3′) | SEQ ID NO | REGION | TARGET SITE | % INHIB |
| 105169 | CAATCGCCACTGAACACAGC | 371 | exon | 821 | 0 |
| | | | Intron: | | |
| 105170 | GTGCTTACCTGGATCAGCAG | 372 | Exon 2 | 881 | 0 |
| | | | junction | | |
| 105171 | GCACATTTGCCTACAAGGAA | 3733 | splice | 1004 | 40 |
| | | | site | | |
| 105172 | TAGAGGGGACACCGTCAGAG | 374 | Intron | 341 | 2 |
| 105173 | TGCGAACGGGCAGAGGCTGG | 375 | Intron | 371 | 0 |
| 105174 | CAACAAAACCTCCGCAAAGC | 376 | Intron | 451 | 0 |
| 105175 | ACCTCCCGCGCCGAAGCGGC | 377 | Intron | 601 | 0 |
| 105176 | CTACGCGCAGCGTTCACACT | 378 | Intron | 651 | 0 |
| 105177 | CTAAAGCTACAAGCAAGTCG | 379 | Intron | 901 | 0 |

As shown in Table 45, SEQ ID NO 373 demonstrated at least 40% inhibition of human mdm2 expression in this assay and is therefore preferred.

### Example 45

2,2′-anhydro[1-(-D-arabinofuranosyl)-5-methyluridine]

5-Methyluridine (ribosylthymine, commercially available through Yamasa, Choshi, Japan) (72.0 g, 0.279 mol), diphenylcarbonate (90.0 g, 0.420 mol) and sodium bicarbonate (2.0 g, 0.024 mol) were added to dimethylformamide (300 mL). The mixture was heated to reflux with stirring allowing the resulting carbon dioxide gas to evolve in a controlled manner. After 1 hour, the slightly darkened solution was concentrated under reduced pressure. The resulting syrup was poured into stirred diethyl ether (2.5 L). The product formed a gum. The ether was decanted and the residue was dissolved in a minimum amount of methanol (ca 400 Ml). The solution was poured into fresh ether as above (2.5 L) to give a stiff gum. The ether was decanted and the gum was dried in a vacuum oven (60° C. at 1 mm Hg for 24 h) to give a solid which was crushed to a light tan powder (57 g, 85% crude yield). NMR was consistent with structure and contamination with phenol and its sodium salt (ca 5%). The material was used as is for ring opening. It can be purified further by column chromatography using a gradient of methanol in ethyl acetate (10-25%) to give a white solid, mp 222-4° C.

### Example 46

1-(2-fluoro- -D-erythro-pentofuranosyl)-5-methyluridine

2,2′-Anhydro[1-(-D-arabinofuranosyl)-5-methyluridine] (71 g, 0.32 mmol) and dioxane (700 mL) are placed in a 2 liter stainless steel bomb and HF/pyridine (100 g, 70%) was added. The mixture was heated for 16 hours at 120-125° C. and then cooled in an ice bath. The bomb was opened and the mixture was poured onto 3 liters of ice. To this mixture was added cautiously sodium hydrogen carbonate (300 g) and saturated sodium bicarbonate solution (400 mL). The mixture was filtered and the filter cake was washed with water (2×100 mL) and methanol (2×500 mL). The water and methanol washes were concentrated to dryness in vacuo. Methanol (200 mL) and coarse silica gel (80 g) were added to the residue and the mixture was concentrated to dryness in vacuo. The resulting material was concentrated onto the silica gel and purified by silica gel column chromatography using a gradient of ethyl acetate and methanol (100:0 to 85:15). Pooling and concentration of the product fractions gave 36.9 g (51%, 2 step yield) of the title compound.

Also isolated from this reaction was 1-(2-phenyl- -D-erythro-pentofuranosyl)-5-methyluridine (10.3 g). This material is formed from the phenol and its sodium salt from the anhydro reaction above when the bomb reaction is carried out on impure material. When the anhydro material is purified this product is not formed. The formed 1-(2-phenyl- -D-erythro-pentofuranosyl)-5-methyluridine was converted into its DMT/phosphoramidite using the same reaction conditions as for the 2′-fluoro material.

### Example 47

1-(5-O-Dimethoxytrityl-2-fluoro- -D-erythro-pentofuranosyl)-5-methyluridine

1-(2-fluoro- -D-erythro-pentofuranosyl)-5-methyluridine (31.15 g, 0.12 mol) was suspended in pyridine (150 mL) and dimethoxytrityl chloride (44.62 g, 0.12 mol) was added. The mixture was stirred in a closed flask for 2 hours and then methanol (30 mL) was added. The mixture was concentrated in vacuo and the resulting residue was partitioned between saturated bicarbonate solution (500 mL) and ethyl acetate (3×500 ml). The ethyl acetate fractions were pooled and dried over magnesium sulfate, filtered and concentrated in vacuo to a thick oil. The oil was dissolved in dichloromethane (100 mL), applied to a silica gel column and eluted with ethyl acetate:hexane:triethylamine, 60/39/1 increasing to 75/24/1. The product fractions were pooled and concentrated in vacuo to give 59.9 g (89%) of the title compound as a foam.

### Example 48

1-(5-O-Dimethoxytrityl-2-fluoro-3-O-N,N-diisopropylamino-2-cyanoethylphosphite- -D-erythro-pentofuranosyl)-5-methyluridine

1-(5-O-Dimethoxytrityl-2-fluoro- -D-erythro-pento-furanosyl)-5-methyluridine (59.8 g, 0.106 mol) was dissolved in dichloromethane and 2-cyanoethyl N,N,N′,N′-tetra-isopropylphosphorodiamidite (46.9 mL, 0.148 mol) and diiso-propylamine tetrazolide (5.46 g, 0.3 eq.) was added. The mixture was stirred for 16 hours. The mixture was washed with saturated sodium bicarbonate (1 L) and the bicarbonate solution was back extracted with dichloromethane (500 mL). The combined organic layers were washed with brine (1 L) and the brine was back extracted with dichloromethane (100 mL). The combined organic layers were dried over sodium sulfate, filtered, and concentrated to a vol of about 200 mL. The resulting material was purified by silica gel column chromatography using hexane/ethyl acetate/triethyl amine 60/40/1. The product fractions were concentrated in vacua, dissolved in acetonitrile (500 ml), filtered, concentrated in vacua, and dried to a foam. The foam was chopped and dried for 24 hour to a constant weight to give 68.2 g (84%) of the title compound. 1H NMR: (CDCl3) 0.9-1.4 (m, 14 H, 4×CH3, 2×CH), 2.3-2.4 (t, 1 H, CH2CN), 2.6-2.7 (t, 1 H, CH2CN), 3.3-3.8 (m, 13 H, 2×CH3OAr, 5′ CH2, CH2OP, C-5 CH3), 4.2-4.3 (m, 1 H, 4′), 4.35-5.0 (m, 1 H, 3′), 4.9-5.2 (m, 1 H, 2′), 6.0-6.1 (dd, 1 H, 1′), 6.8-7.4 (m, 13 H, DMT), 7.5-7.6 (d, 1 H, C-6), 8.8 (bs, 1 H, NH). 31P NMR (CDC13); 151.468, 151.609, 151.790, 151.904.

### Example 49

1-(3′,5′-di-O-acetyl-2-fluoro- -D-erythro-pentofuranosyl)-5-methyluridine

1-(2-fluoro- -D-erythro-pentofuranosyl)-5-methyluridine (22.4 g, 92 mmol, 85% purity), prepared as per the procedure of Example 2, was azeotroped with pyridine (2×150 mL) and dissolved in pyridine (250 mL). Acetic anhydride (55 mL, 0.58 mol) was added and the mixture was stirred for 16 hours. Methanol (50 mL) was added and stirring was continued for 30 minutes. The mixture was evaporated to a syrup. The syrup was dissolved in a minimum amount of methanol and loaded onto a silica gel column. Hexane/ethyl acetate, 1:1, was used to elute the product fractions. Purification gave 19.0 g (74%) of the title compound.

### Example 50

4-Triazine-1-(3′,5′-di-O-acetyl-2-fluoro- -D-erythro-pentofuranosyl)-5-methyluridine

1,2,4-Triazole (106 g, 1.53 mol) was dissolved in acetonitrile (150 mL) followed by triethylamine (257 mL, 1.84 mol). The mixture was cooled to between 0 and 10° C. using an ice bath. POCl3 (34.5 mL, .375 mol) was added slowly via addition funnel and the mixture was stirred for an additional 45 minutes. In a separate flask, 1-(3′,5′-Di-O-acetyl-2-fluoro- -D-erythro-pentofuranosyl)-5-methyluridine (56.9 g, .144 mol) was dissolved in acetonitrile (150 mL). The solution containing the 1-(3′,5′-Di-O-acetyl-2-fluoro- -D-erythro-pentofuranosyl)-5-methyluridine was added via cannula to the triazole solution slowly. The ice bath was removed and the reaction mixture was allowed to warm to room temperature for 1 hour. The acetonitrile was removed in vacuo and the residue was partitioned between saturated sodium bicarbonate solution (400 mL) and dichloromethane (4×400 mL). The organic layers were combined and concentrated in vacuo. The resulting residue was dissolved in ethyl acetate (200 mL) and started to precipitate a solid. Hexanes (300 mL) was added and additional solid precipitated. The solid was collected by filtration and washed with hexanes (2×200 mL) and dried in vacuo to give 63.5 g which was used as is without further purification.

### Example 51

5-methyl-1-(2-fluoro- -D-erythro-pentofuranosyl)-Cytosine

4-Triazine-1-(3′,5′-di-O-acetyl-2-fluoro- -D-erythro-pentofuranosyl)-Thymine (75.5 g, .198 mol) was dissolved in ammonia (400 mL) in a stainless steel bomb and sealed overnight. The bomb was cooled and opened and the ammonia was evaporated. Methanol was added to transfer the material to a flask and about 10 volumes of ethyl ether was added. The mixture was stirred for 10 minutes and then filtered. The solid was washed with ethyl ether and dried to give 51.7 g (86%) of the title compound.

### Example 52

4-N-Benzoyl-5-methyl-1-(2-fluoro- -D-erythro-pentofuranosyl)-Cytosine

5-methyl-1-(2-fluoro- -D-erythro-pentofuranosyl)-Cytosine (54.6 g, 0.21 mol) was suspended in pyridine (700 mL) and benzoic anhydride (70 g, .309 mol) was added. The mixture was stirred for 48 hours at room temperature. The pyridine was removed by evaporation and methanol (800 mL) was added and the mixture was stirred. A precipitate formed which was filtered, washed with methanol (4×50mL), washed with ether (3×100 mL), and dried in a vacuum oven at 45° C. to give 40.5 g of the title compound. The filtrate was concentrated in vacuo and treated with saturated methanolic ammonia in a bomb overnight at room temperature. The mixture was concentrated in vacuo and the resulting oil was purified by silica gel column chromatography. The recycled starting material was again treated as above to give an additional 4.9 g of the title compound to give a combined 45.4 g (61%) of the title compound.

### Example 53

4-N-Benzoyl-5-methyl-1-(2-fluoro-5-O-dimethoxytrityl- -D-erythro-pentofuranosyl)-Cytosine

4-N-Benzoyl-5-methyl-1-(2-fluoro- -D-erythro-pentofuranosyl)-Cytosine (45.3 g, 0.124 mol) was dissolved in 250 ml dry pyridine and dimethoxytrityl chloride (46.4 g, 0.137 mol) was added. The reaction mixture was stirred at room temperature for 90 minutes and methanol (20 mL) was added. The mixture was concentrated in vacuo and partitioned between ethyl acetate (2×1 L) and saturated sodium bicarbonate (1 L). The ethyl acetate layers were combined, dried over magnesium sulfate and evaporated in vacuo. The resulting oil was dissolved in dichloromethane (200 mL) and purified by silica gel column chromatography using ethyl acetate/hexane/triethyl amine 50:50:1. The product fractions were pooled concentrated in vacuo dried to give 63.6 g (76.6%) of the title compound.

### Example 54

4-N-Benzoyl-5-methyl-1-(2-fluoro-3-O-N,N-diisopropylamino-2-cyanoethylphosphite-5-O-dimethoxytrityl- -D-erythro-pentofuranosyl)-Cytosine

4-N-Benzoyl-5-methyl-1-(2-fluoro-5-O-dimethoxytrityl- -D-erythro-pentofuranosyl)-Cytosine (61.8 g, 92.8 mmol) was stirred with dichloromethane (300 mL), 2-cyanoethyl N,N,N′,N′-tetraisopropylphosphorodiamidite (40.9 mL, 0.130 mol) and diisopropylamine tetrazolide (4.76 g, 0.3 eq.) at room temperature for 17 hours. The mixture was washed with saturated sodium bicarbonate (1 L) and the bicarbonate solution was back extracted with dichloromethane (500 mL). The combined organic layers were washed with brine (1 L) and the brine was back extracted with dichloromethane (100 mL). The combined organic layers were dried over sodium sulfate, filtered, and concentrated to a vol of about 200 mL. Tht resulting material was purified by silica gel column chromatography using hexane/ethyl acetate/triethyl amine 60/40/1. The product fractions were concentrated in vacuo, dissolved in acetonitrile (500 ml), filtered, concentrated in vacuo, and dried to a foam. The foam was chopped and dried for 24 hours to a constant weight to give 72.4 g (90%) of the title compound. 1H NMR: (CDCl3) 1.17-1.3 (m, 12 H, 4×CH3), 1.5-1.6 (m, 2 H, 2×CH), 2.3-2.4 (t, 1 H, CH2CN), 2.6-2.7 (t, 1 H, CH2CN), 3.3-3.9 (m, 13 H, 2×CH3OAr, 5′ CH2, CH2OP, C-5 CH3), 4.2-4.3 (m, 1 H, 4′), 4.3-4.7 (m, 1 H, 3′), 5.0-5.2 (m, 1 H, 2′), 6.0-6.2 (dd, 1 H, 1′), 6.8-6.9 (m, 4 H, DMT), 7,2-7.6 (m, 13 H, DMT, Bz), 7.82-7.86 (d, 1 H, C-6), 8.2-8.3 (d, 2 H, Bz). 31P NMR (CDC13); bs, 151.706; bs, 151.941.

### Example 55

1-(2,3-di-O-butyltin- -D-erythro-Pentofuranosyl)-5-Methyluridine

5-Methyl uridine (7.8 g, 30.2 mmol) and dibutyltin oxide (7.7 g, 30.9 mmol) were suspended in methanol (150 mL) and heated to reflux for 16 hours. The reaction mixture was cooled to room temperature, filtered, and the solid washed with methanol (2×150 mL). The resulting solid was dried to give 12.2 g (80.3%) of the title compound. This material was used without further purification in subsequent reactions. NMR was consistent with structure.

### Example 56

1-(2-O-Propyl- -D-erythro-Pentofuranosyl)-5-Methyluridine

1-(2,3-di-O-butyltin- -D-erythro-pentofuranosyl)-5-methyluridine (5.0 g, 10.2 mmol) and iodopropane (14.7 g, 72.3 mmol) were stirred in DMF at 100° C. for 2 days. The reaction mixture was cooled to room temperature and filtered and concentrated. The residual DMF was coevaporated with acetonitrile. After drying the residue there was obtained 2.40 g (78%) of the title compound and the 3′-O-propyl isomer as a crude mixture. This material was used without further purification in subsequent reactions.

### Example 57

1-(2-O-Propyl-5-O-Dimethoxytrityl- -D-erythro-Pentofuranosyl)-5-Methyluridine

1-(2-O-Propyl- -D-erythro-pentofuranosyl)-5-methyluridine and the 3′-O-propyl isomer as a crude mixture (2.4 g, 8.4 mmol) was coevaporated with pyridine (2×40 mL) and dissolved in pyridine (60 mL). The solution was stirred at room temperature under argon for 15 minutes and dimethoxytrityl chloride (4.27 g, 12.6 mmol) was added. The mixture was checked periodically by tlc and at 3 hours was completed. Methanol (10 mL) was added and the mixture was stirred for 10 minutes. The reaction mixture was concentrated in vacuo and the resulting residue purified by silica gel column chromatography using 60:40 hexane/ethyl acetate with 1% triethylamine used throughout. The pooling and concentration of appropriate fractions gave 1.32 g (26%) of the title compound.

### Example 58

1-(2-O-Propyl-3-O-N,N-Diisopropylamino-2-Cyanoethylphosphite-5-O-Dimethoxytrityl- -D-erythro-Pentofuranosyl)-5-Methyluridine

1-(2-O-Propyl-5-O-dimethoxytrityl- -D-erythro-pento-furanosyl)-5-methyluridine (50.0 g, 86 mmol), 2-cyanoethyl-N,N,N′,N′-tetra-isopropylphosphorodiamidite (38 mL, 120 mmol), and diisopropylamine tetrazolide (4.45 g, 25.8 mmol) were dissolved in dichloromethane (500 mL) and stirred at room temperature for 40 hours. The reaction mixture was washed with saturated sodium bicarbonate solution (2×400 mL) and brine (1×400 mL). The aqueous layers were back extracted with dichloromethane. The dichloromethane layers were combined, dried over sodium sulfate, filtered, and concentrated in vacuo. The resultant residue was purified by silica gel column chromatography using ethyl acetate/hexane 40:60 and 1% triethylamine. The appropriate fractions were pooled, concentrated, and dried under high vacuum to give 43 g (67%).

### Example 59

1-(2-O-Propyl-3-O-Acetyl-5-O-Dimethoxytrityl- -D-erythro-Pentofuranosyl)-5-Methyluridine

1-(2-O-Propyl-5-dimethoxytrityl- -D-erythro-pentofuranosyl)-5-methyluridine (10.0 g, 16.6 mmol) was dissolved in pyridine (50 mL) and acetic anhydride (4.7 ml, 52.7 mmol) was added. The reaction mixture was stirred for 18 hours and excess acetic anhydride was neutralized with methanol (10 mL). The mixture was concentrated in vacuo and the resulting residue dissolved in ethyl acetate (150 mL). The ethyl acetate was washed with saturated NaHCO3 (150 mL) and the saturated NaHCO3 wash was back extracted with ethyl acetate (50 mL). The ethyl acetate layers were combined and concentrated in vacuo to yield a white foam 11.3 g. The crude yield was greater than 100% and the NMR was consistent with the expected structure of the title compound. This material was used without further purification in subsequent reactions.

### Example 60

1-(2-O-Propyl-3-O-Acetyl-5-O-Dimethoxytrityl- -D-erythro-Pentofuranosyl)-4-Triazolo-5-Methylpyrimidine

Triazole (10.5 g, 152 mmol) was dissolved in acetonitrile (120 ml) and triethylamine (23 mL) with stirring under anhydrous conditions. The resulting solution was cooled in a dry ice acetone bath and phosphorous oxychloride (3.9 mL, 41 mmol) was added slowly over a period of 5 minutes. The mixture was stirred for an additional 10 minutes becoming a thin slurry indicative of product formation. 1-(2-O-Propyl-3-O-acetyl-5-O-dimethoxytrityl- -D-erythro-pentofuranosyl)-5-methyluridine (11.2 g, 165 mmol) was dissolved in acetonitrile (150 mL) and added to the slurry above, maintaining dry ice acetone bath temperatures. The reaction mixture was stirred for 30 minutes and then allowed to warm to room temperature and stirred for an additional 2 hours. The mixture was placed in a freezer at 0° C. for 18 hours and then removed and allowed to warm to room temperature. Tlc in ethyl acetate/hexane 1:1 of the mixture showed complete conversion of the starting material. The reaction mixture was concentrated in vacuo and redissolved in ethyl acetate (300 mL) and extracted with saturated sodium bicarbonate solution (2×400 mL) and brine (400 mL). The aqueous layers were back extracted with ethyl acetate (200 mL). The ethyl acetate layers were combined, dried over sodium sulfate, and concentrated in vacuo. The crude yield was 11.3 g (95%). The NMR was consistent with the expected structure of the title compound. This material was used without further purification in subsequent reactions.

### Example 61

1-(2-O-Propyl-5-O-Dimethoxytrityl- -D-erythro-Pentofuranosyl)-5-Methylcytidine

1-(2-O-Propyl-3-O-acetyl-5-O-dimethoxytrityl- -D-erythro-pentofuranosyl)-4-triazolo-5-methylpyrimidine

(11.2 g, 16.1 mmol) was dissolved in liquid ammonia (50 mL) in a 100 mL bomb at dry ice acetone temperatures. The bomb was allowed to warm to room temperature for 18 hours and then recooled to dry ice acetone temperatures. The bomb contents were transferred to a beaker and methanol (50 mL) was added. The mixture was allowed to evaporate to near dryness. Ethyl acetate (300 mL) was added and some solid was filtered off prior to washing with saturated sodium bi-carbonate solution (2×250 mL). The ethyl acetate layers were dried over sodium sulfate, filtered, combined with the solid previously filtered off, and concentrated in vacuo to give 10.1 g of material. The crude yield was greater than 100% and the NMR was consistent with the expected structure of the title compound. This material was used without further purification in subsequent reactions.

### Example 62

1-(2-O-Propyl-5-O-Dimethoxytrityl- -D-erythro-Pentofuranosyl)-4-N-Benzoyl-5-Methylcytidine

1-(2-O-Propyl-5-O-dimethoxytrityl- -D-erythro-pentofuranosyl)-5-methylcytidine (7.28 g, 10.1 mmol) and benzoic anhydride (4.5 g, 20 mmol) were dissolved in DMF (60 mL) and stirred at room temperature for 18 hours. The reaction mixture was concentrated in vacuo and redissolved in ethyl acetate (300 mL). The ethyl acetate solution was washed with saturated sodium bicarbonate solution (2×400 mL), dried over sodium sulfate, filtered, and concentrated in vacuo. The residue was purified by silica gel column chromatography using ethyl acetate/hexane 1:2 and 1% triethylamine. The appropriate fractions were pooled, concentrated, and dried under high vacuum to give 5.1 g (59% for 4 steps starting with the 1-(2-O-Propyl-dimethoxytrityl- -D-erythro-pentofuranosyl)-5-methyluridine).

### Example 63

1-(2-O-Propyl-3-O-N,N-Diisopropylamino-2-Cyanoethylphosphite-5-O-Dimethoxytrityl- -D-erythro-Pentofuranosyl)-4-N-Benzoyl-5-Methylcytidine

1-(2-O-Propyl-5-O-dimethoxytrityl- -D-erythro-pentofuranosyl)-4-N-benzoyl-5-methylcytidine (5.0 g, 7 mmol), 2-cyanoethyl-N,N,N′,N′-tetra-isopropylphosphorodiamidite (3.6 mL, 11.3 mmol), and diisopropylaminotetrazolide (0.42 g, 2.4 mmol) were dissolved in dichloromethane (80 mL) and stirred at room temperature for 40 hours. The reaction mixture was washed with saturated sodium bicarbonate solution (2×40 mL) and brine (1×40 mL). The aqueous layers were back extracted with dichloromethane. The dichloromethane layers were combined, dried over sodium sulfate, filtered, and concentrated in vacuo. The resultant residue was purified by silica gel column chromatography using ethyl acetate/hexane 40:60 and 1% triethylamine. The appropriate fractions were pooled, concentrated, and dried under high vacuum to give 7.3 g (98%).

### Example 64

2,6-Dichloro-9-(2-deoxy-3,5-di-O-p-toluoyl- -D-erythro-pentofuranosyl)purine.

To a stirred solution of 2,6-dichloropurine (25.0 g, 132.27 mmol) in dry acetonitrile (1000 mL) was added sodium hydride (60% in oil, 5.40 g, 135 mmol) in small portions over a period of 30 minutes under argon atmosphere. After the addition of NaH, the reaction mixture was allowed to stir at room temperature for 30 minutes. Predried and powdered 1-chloro-2′-deoxy-3,5,di-O-p-toluoyl- -D-erythro-pentofuranose (53.0 g, 136 mmol) was added during a 15 minute period and the stirring continued for 10 hours at room temperature over argon atmosphere. The reaction mixture was evaporated to dryness and the residue dissolved in a mixture of CH₂Cl₂/H₂O (250:100 mL) and extracted in dichloromethane (2×250mL). The organic extract was washed with brine (100 mL), dried, and evaporated to dryness. The residue was dissolved in dichloromethane (300 mL), mixed with silica gel (60-100 mesh, 250 g) and evaporated to dryness. The dry silica gel was placed on top of a silica gel column (250−400 mesh, 12×60 cm) packed in hexane. The column was eluted with hexanes (1000 mL), toluene (2000 mL), and toluene:ethyl acetate (9:1, 3000 mL). The fractions having the required product were pooled together and evaporated to give 52 g (72%) of 3 as white solid. A small amount of solid was crystallized from ethanol for analytical purposes. mp 160-162° C.; ¹H NMR (DMSO-d₆); 2.36 (s, 3 H, CH₃), 2.38 (s, 3 H, CH₃), 2.85 (m, 1 H, C₂′H), 3.25 (m, 1 H, C₂′H), 4.52 (m, 1 H, C₄H), 4.62 (m, 2 H, C₅,CH₂), 5.80 (m, 1 H, C₃′H), 6.55 (t, 1 H, J_{1′,2′}=6.20 Hz, C_{1′}H), 7.22 (dd, 2 H, ArH), 7.35 (dd, 2 H, ArH), 7.72 (dd, 2 H, ArH), 7.92 (dd, 2 H, ArH), and 8.92 (S, 1 H, C₈H)

### Example 64

2-Chloro-6-allyloxy-9-(2′-deoxy- -D-erythro-pentofuranosyl)purine. (2)

To a stirred suspension of 2,6-dichloro-9-(2′-deoxy-3′,5′-di-O-p-toluoyl- -D-erythro-pentofuranosyl)-purine (1, 10.3 g, 19.04 mmol) in allyl alcohol (150 mL) was added sodium hydride (60%, 0.8 g, 20.00 mmol) in small portions over a 10 minute period at room temperature. After the addition of NaH, the reaction mixture was placed in a preheated oil bath at 55° C. The reaction mixture was stirred at 55° C. for 20 minutes with exclusion of moisture. The reaction mixture was cooled, filtered, and washed with allyl alcohol (50 mL). To the filtrate IRC-50 (weakly acidic) H⁺ resin was added until the pH of the solution reached 4-5. The resin was filtered, washed with methanol (100 mL), and the filtrate was evaporated to dryness. The residue was absorbed on silica gel (log, 60-100 mesh) and evaporated to dryness. The dried silica gel was placed on top of silica column (5×25 cm, 100-250 mesh) packed in dichloromethane. The column was then eluted with CH₂Cl₂/acetone (1:1). The fractions having the product were pooled together and evaporated to dryness to give 6 g (96%) of the title compound as foam. ¹H NMR (Me₂SO-d₆) 2.34 (m, 1 H, C_{2′}H), 2.68 (m, 1 H, C_{2′}H), 3.52 (m, 2 H, C_{5′}H), 3.86 (m, 1 H, C_{4′}H), 4.40 (m, 1 H, C_{3′}H), 4.95 (t, 1 H, C_{5′}OH), 5.08 (d, 2 H, CH₂), 5.35 (m, 3 H, CH₂ and C_{3′}OH), 6.10 (m, 1 H, CH), 6.35 (t, 1 H, J_{1′,2′}=6.20 Hz, C_{1′}H), 8.64 (s, 1 H, C₈H) . Anal. Calcd for C₁₃H₁₅ClN₄O₄: C, 47.78; H, 4.63; N, 17.15; Cl, 10.86. Found: C, 47.58; H, 4.53; N, 17.21; Cl, 10.91.

### Example 65

2-Chloro-9-(2′-deoxy- -D-erythro-pentofuranosyl)inosine. (3)

A mixture of 2 (6 g, 18.4 mmol), Pd/C (10%, 1 g) and triethylamine (1.92 g, 19.00 mmol) in ethyl alcohol (200 mL) was hydrogenated at atmospheric pressure during 30 minute periods at room temperature. The reaction mixture was followed by the absorption of volume of hydrogen. The reaction mixture was filtered, washed with methanol (50 mL), and the filtrate evaporated to dryness. The product 5.26 g (100%) was found to be moisture sensitive and remained as a viscous oil. The oil was used as such for further reaction without purification. A small portion of the solid was dissolved in water and lyophilized to give an amorphous solid: ¹H NMR (Me₂SO-d₆) 2.35 (m, 1 H, C_{2′}H), 2.52 (m, 1 H, C_{2′}H), 3.54 (m, 2 H, C_{5′}H), 3.82 (m, 1 H, C_{4′}H), 4.35 (m, 1 H, C_{3′}H), 4.92 (b s, 1 H, C_{5′}OH), 5.35 (s, 1 H, C_{3′}OH), 6.23 (t, 1 H, J_{1′,2′}=6.20 Hz, C_{1′}H), 8.32 (s, 1 H, C₈H), 13.36 (b s, 1 H, NH).

### Example 66

N₂-[Imidazol-1-yl(propyl)]-9-(2′-deoxy- -D-erythro-pentofuranosyl)guanosine. (4)

A solution of the nucleoside of 3 (10.3 g, 36.00 mmol) and 1-(3-aminopropyl)imidazole (9.0 g, 72.00 mmol) in 2-methoxyethanol (60 mL) was heated in a steel bomb at 100° C. (oil bath) for 24 hours. The bomb was cooled to 0° C., opened carefully and the precipitated solid was filtered. The solid was washed with methanol (50 mL), acetone (50 mL), and dried over sodium hydroxide to give 9 g (67%) of pure 4. A small amount was recrystallized from DMF for analytical purposes: mp 245-47° C.: ¹H NMR (Me₂SO-d₆) 1.94 (m, 2 H, CH₂), 2.20 (m, 1 H, C_{2′}H), 2.54 (m, 1 H, C_{2′}H), 3.22 (m, 2 H, CH₂), 3.51 (m, 2 H, C_{5′}H), 3.80 (m, 1 H, C_{4′}H), 3.98 (m, 2 H, CH₂), 4.34 (m, 1 H, C_{3′}H), 4.90 (b s, 1 H, C_{5′}OH), 5.51 (s, 1 H, C_{3′}OH), 6.12 (t, 1 H, J_{1′,2′}=6.20 Hz, C_{1′}H), 6.46 (b s, 1 H, NH), 6.91 (s, 1 H, ImH), 7.18 (s, 1 H, ImH), 7.66 (s, 1 H, ImH), 7.91 (s, 1 H, C₈H), 10.60 (b s, 1 H, NH). Anal. Calcd for C₁₆H₂₁N₇O₄: C, 51.19; H, 5.64; N, 26.12. Found: C, 50.93; H, 5.47; N, 26.13.

### Example 67

N₂-3′,5′-Tri-O-isobutyryl-N₂-[imidazol-1-yl(propyl)]-9-(2′-deoxy- -D-erythro-pentofuranosyl)guanosine. (5)

To a well dried solution of the substrate of 4 (1.5 g, 4.00 mmol) and triethylamine (1.62 g, 16.00 mmol) in dry pyridine (30 mL) and dry DMF (30 mL) was added isobutyryl chloride (1.69 g, 16.00 mmol) at room temperature. The reaction mixture was allowed to stir at room temperature for 12 hours and evaporated to dryness. The residue was partitioned between dichloromethane (100 mL) and water (50 mL) and extracted with CH₂Cl₂ (2×200 mL). The organic extract was washed with brine (100 mL) and dried over anhydrous MgSO₄. The dried organic extract was evaporated to dryness and the residue was purified over flash chromatography using CH₂Cl₂/MeOH as eluent. The pure fractions were pooled, evaporated to dryness which on crystallization from CH₂Cl₂/MeOH gave 1.8 g (77%) of 5 as a colorless crystalline solid: mp 210-212° C.; ¹H NMR (Me₂SO-d6) 1.04 (m, 18 H, 3 Isobutyryl CH₃), 1.94 (m, 2 H, CH₂), 2.56 (m, 4 H, C₂′H and 3 Isobutyryl CH) 2.98 (m, 1 H, C₂′H), 3.68 (m, 2 H, CH₂), 3.98 (m, 2 H, CH₂), 4.21 (2 m, 3 H, C_{5′}H and C_{4′}H), 5.39 (m, 1 H, C_{3′}H), 6.30 (t, 1 H, J_{′,2′}=6.20 Hz, C_{1′}H), 6.84 (s, 1 H, ImH), 7.18 (s, 1 H, ImH), 7.34 (s, 1 H, ImH), 8.34 (s, 1 H, C₈H), 10.60 (b s, 1 H, NH). Anal. Calcd for C₂₈H₃₉N₇O₇: C, 57.42; H, 6.71; N, 16.74. Found: C, 57.29; H, 6.58; N, 16.56.

### Example 68

6-O-[2-(4-Nitrophenyl)ethyl]-N₂-3′,5′-tri-O-isobutyryl-N₂-[imidazol-1-yl(propyl)]-9-(2′-deoxy- -D-erythro-pentofuranosyl)guanosine. (6)

To a stirred solution of 5 (2.0 g, 3.42 mmol), triphenylphosphine (2.68 g, 10.26 mmol) and p-nitrophenyl ethanol (1.72 g, 10.26 mmol) in dry dioxane was added diethylazodicarboxylate (1.78 g, 10.26 mmol) at room temperature. The reaction mixture was stirred at room temperature for 12 hours and evaporated to dryness. The residue was purified by flash chromatography over silica gel using CH₂Cl₂/acetone as the eluent. The pure fractions were pooled together and evaporated to dryness to give 2.4 g (96%) of the title compound as an amorphous solid. ¹H NMR (Me₂SO-d₆) 1.04 (m, 18 H, 3 Isobutyryl CH₃), 1.94 (m, 2 H, CH₂), 2.50 (m, 3 H, C₂ H and 2 Isobutyryl CH), 3.00 (m, 1 H, C_{2′}H), 3.12 (m, 1 H, Isobutyryl CH), 3.24 (m, 2 H, CH₂), 3.82 (m, 2 H, CH₂), 3.98 (m, 2 H, CH₂), 4.21 (2 m, 3 H, C_{5′}CH₂ and C_{4′}H), 4.74 (m, 2 H, CH₂), 5.39 (m, 1 H, C_{3′}H) 6.34 (t, 1 H, J_{1′,2′}=6.20 Hz, C1′H), 6.82 (s, 1 H, ImH), 7.08 (s, 1 H, ImH), 7.56 (s, 1 H, ImH), 7.62 (d, 2 H, ArH), 8.1 (d, 2 H, ArH), 8.52 (s, 1 H, C₈H) . Anal. Calcd for C₃₆H₄₆N₈O₉-½ H₂O: C, 58.13; H, 6.37; N, 15.01. Found: C, 58.33; H, 6.39; N, 14.75.

### Example 69

6-O- [2-(4-Nitrophenyl) -ethyl]-N₂-isobutyryl-N₂-[imidazol-1-yl-(propyl)]-9-(2′-deoxy- -D-erythro-pentofuranosyl)guanosine. (7)

To a stirred solution of 6 (9.00 g, 12.26 mmol) in methanol (250 ml) was treated with ammonium hydroxide (30%, 150 ml) at room temperature. The reaction mixture was stirred at room temperature for 4 hours and evaporated to dryness under reduced pressure. The residue was purified by flash chromatography over silica gel using CH₂Cl₂/MeOH as the eluent. The pure fractions were pooled together and evaporated to dryness to give 5.92 g (81%) of the title compound: ¹H NMR (Me₂SO-d₆) 1.04 (m, 6H, Isobutyryl CH₃), 1.96 (m, 2 H, CH₂), 2.32 (m, 1 H, C_{2′}H), 2.62 (m, 1 H, C_{2′}H), 3.14 (m, 1 H, Isobutyryl CH), 3.26 (m, 2 H, CH₂), 3.52 (m, 2 H, C_{5′}CH₂), 3.82 (m, 3 H, CH₂ and C_{4′}H), 3.96 (m, 2 H, CH₂), 4.36 (m, 1 H, C_{3′}H), 4.70 (m, 2 H, CH₂), 4.96 (b s, 1 H, C_{5′}OH), 5.42 (b s, 1 H, C_{3′}OH), 6.34 (t, 1 H, J_{1′,2′}=6.20 Hz, C_{1′}H), 6.82 (s, 1 H, ImH), 7.12 (s, 1 H, ImH), 7.54 (s, 1 H, ImH), 7.62 (d, 2 H, ArH), 8.16 (d, 2 H, ArH), 8.56 (s, 1 H, C₈H). Anal. Calcd for C₂₈H₃₄N₈O₇-1/2 H₂O: C, 55.71; H, 5.84; N, 18.56. Found: C, 55.74; H, 5.67; N, 18.43.

### Example 70

5α-O-(4,4α-Dimethoxytrityl)-6-O-[2-(4-nitrophenyl)ethyl]-N₂-isobutyryl-N₂-[imidazol-1-yl (propyl)]-2α-deoxy- -D-erythro-pentofuranosyl)guanosine. (8)

The substrate 7 (5.94 g, 10 mmol), was dissolved in dry pyridine (75 mL) and evaporated to dryness. This was repeated three times to remove traces of moisture. To this well dried solution of the substrate in dry pyridine (100 mL) was added dry triethylamine (4.04 g, 40 mmol), 4-(dimethylamino)pyridine (1.2 g, 30 mmol) at room temperature. The reaction mixture was stirred at room temperature for 12 hours under argon atmosphere. Methanol (50 mL) was added and the stirring was continued for 15 minutes and evaporated to dryness. The residue was purified by flash chromatography over silica gel using dichloromethane-acetone containing 1% triethylamine as the eluent. The pure fractions were pooled together and evaporated to dryness to give 7.2 g (80%) of the title compound as a colorless foam: ¹H NMR (Me₂SO-d₆) 1.04 (m, 6 H, Isobutyryl CH₃), 1.94 (m, 2 H, CH₂), 2.34 (m, 1 H, C_{2′}H), 2.80 (m, 1 H, C_{2′}H), 3.04 (m, 1 H, Isobutyryl CH), 3.18 (m, 2 H, CH₂), 3.28 (m, 2 H, CH₂), 3.62 (s, 3 H, OCH₃), 3.66 (s, 3 H, OCH₃), 3.74 (2 m, 2 H, C_{5′}CH₂), 3.98 (m, 3 H, CH₂ and C_{4′}H), 4.36 (m, 1 H, C_{3′}H) 4.70 (m, 2 H, CH₂), 5.44 (b s, 1 H, C3′OH), 6.32 (t, 1 H, J_{1′,2′}=6.20 Hz C₁ H), 6.64-7.32 (m, 15 H, ImH and ArH) 7.52 (s, 1 H, ImH), 7.62 (d, 2 H, ArH), 8.16 (d, 2 H, ArH), 8.42 (s, 1 H, C₈H). Anal. Calcd for C₄₉H₅₂N₈O₉- H₂0: C, 64.32; H, 5.95; N, 12.25. Found: C, 64.23; H, 5.82; N, 12.60.

### Example 71

3α-O-(N,N-Diisopropylamino) ( -cyanoethoxy)phosphanyl]-5′-0-(4,4′-dimethoxytrityl)-6-0-[2-(4-nitrophenyl)ethyl]-N₂-isobutyryl-N₂- [imidazol-1-yl(propyl)]-9-(2′-deoxy- -D-erythro-pentofuranosyl)guanosine. (9)

The substrate of 8 (2.5 g, 2.7 mmol), was dissolved in dry pyridine (30 mL) and evaporated to dryness. This was repeated three times to remove last traces of water and dried over solid sodium hydroxide overnight. The dried 8 was dissolved in dry dichloromethane (30 mL) and cooled to 0° C. under argon atmosphere. To this cold stirred solution was added N,N-diisopropylethylamine (0.72 g, 5.6 mmol) followed by ( -cyanoethoxy)chloro(N,N-diisopropylamino) phosphate (1.32 g, 5.6 mmol) dropwise over a period of 15 minutes. The reaction mixture was stirred at 0° C. for 1 hour and at room temperature for 2 hours. The reaction mixture was diluted with dichloromethane (100 mL) and washed with brine (50 mL). The organic extract was dried over anhydrous MgSO₄ and the solvent was removed under reduced pressure. The residue was purified by flash chromatography over silica gel using hexane/acetone containing 1% triethylamine as the eluent. The main fractions were collected and evaporated to dryness. The residue was dissolved in dry dichloromethane (10 mL) and added dropwise, into a stirred solution of hexane (1500 mL), during 30 minutes. After the addition, the stirring was continued for an additional 1 hour at room temperature under argon. The precipitated solid was filtered, washed with hexane and dried over solid NaOH under vacuum overnight to give 2.0 g (65%) of the title compound as a colorless powder: ¹H NMR (Me₂SO-d₆) 1.04 (2 m, 18 H, 3 Isobutyryl CH₃), 1.94 (m, 2 H, CH₂), 2.44 (m 3 H, C_{2′}H and 2 Isobutyryl CH), 2.80 (m, 1 H, C_{2′}H), 3.2 (m, 5 H, 2 CH₂ and Isobutyryl CH), 3.44 - 3.98 (m, 12 H, CH₂, 2 OCH₃ and C₅CH₂), 4.16 (m, 1 H, C₄H), 4.64 (m, 3 H, C_{3′}H and CH₂), 6.32 (t, 1 H, J_{1′,2′}=6.20 Hz, C_{1′}H), 6.64-7.32 (m, 16 H, 3 ImH and ArH), 7.44 (d, 2 H, ArH), 8.16 (d, 3 H, ArH and C₈H).

### Example 72

N₂-[Imidazol-1-yl(propyl)]-9-(2′-deoxy- -D-erythro-pentofuranosyl)adenosine. (11)

A suspension of 2-chloro-9-(2′-deoxy- -D-erythro-pentofuranosyl)adenosine (10, 10.68 g, 37.47 mmol) and 1-(3 aminopropyl) imidazole (12.5 g, 100 mmol) in 2-methoxyethanol (80 mL) was heated at 125° C. for 45 hours in a steel bomb. The bomb was cooled to 0° C., opened carefully, and evaporated to dryness. The residue was coevaporated several times with a mixture of ethanol and toluene. The residue was dissolved in ethanol which on cooling gave a precipitate. The precipitate was filtered and dried. The filtrate was evaporated to dryness and the residue carried over to the next reaction without further purification. ¹H NMR (Me₂SO-d₆) 1.94 (m, 2 H, CH₂), 2.18 (m, 1 H, C_{2′}H), 2.36 (m, 1 H, C_{2′}H), 3.18 (m, 2 H, CH₂), 3.52 (2 m, 2 H, C_{5′}CH₂), 3.80 (m, 1 H, C_{4′}H), 4.02 (m, 2 H, CH₂), 4.36 (m, 1 H, C_{3′}H), 5.24 (b s, 2 H, C_{3′}OH and C_{5′}OH), 6.18 (t, 1 H, J_{1′,2′}=6.20 Hz, C_{1′}H), 6.42 (t, 1 H, NH), 6.70 (b s, 2 H NH₂), 6.96 (s, 1 H, ImH), 7.24 (s, 1 H, ImH), 7.78 (s, 1 H, ImH), 7.90 (s, 1 H, C₈H). Anal. Calcd for C₁₆H₂₂N₈O₃: C, 51.33; H, 5.92; N, 29.93. Found: C, 51.30; H, 5.92; N, 29.91.

### Example 73

3′,5′-O-[(Tetraisopropyldisiloxane-1,3-diyl)-N₂-(imidazol-1-yl)(propyl)]-9-(2′-deoxy- -D-erythro-pentofuranosyl) aminoadenosine.

The crude product 11 (14.03 g) was dissolved in dry DMF (100 mL) dry pyridine (50 mL), and evaporated to dryness. This was repeated three times to remove all the water. The dried substrate was dissolved in dry DMF (75 mL) and allowed to stir at room temperature under argon atmosphere. To this stirred solution was added dry triethylamine (10.1 g, 100 mmol) and 1,3-dichloro-1,1, 3,3-tetraisopropyldisiloxane (TipSiCl, 15.75 g, 50.00 mmol) during a 15 minute period. After the addition of TipSiCl, the reaction mixture was allowed to stir at room temperature overnight. The reaction mixture was evaporated to dryness. The residue was mixed with toluene (100 mL) and evaporated again. The residue was purified by flash chromatography over silica gel using CH₂Cl₂/MeOH as eluent. The pure fractions were pooled and evaporated to dryness to give 12.5 g (54%) of 12 as an amorphous powder: ¹H NMR (Me₂SO-d₆) 1.00 (m, 28 H), 1.92 (m, 2 H, CH₂), 2.42 (m, 1 H, C_{2′}H), 2.80 (m, 1 H, C_{2′}H) 3.18 (m, 2 H, CH₂), 3.84 (2 m, 3 H, _{5′}CH₂ and C_{4′}H), 4.00 (t, 2 H, CH₂), 4.72 (m, 1 H, C_{3′}H), 6.10 (m, 1 H, C_{1′}H), 6.48 (t, 1 H, NH), 6.74 (b s, 2 H, NH₂), 6.88 (s, 1 H, ImH), 7.18 (s, 1 H, ImH), 7.64 (s, 1 H, ImH), 7.82 (s, 1 H, C₈H). Anal. Calcd for C₂₈H₅₀N₈O₄Si₂: C, 54.33; H, 8.14; N, 18.11. Found: C, 54.29; H, 8.09; N, 18.23.

### Example 74

3′,5′-O-(Tetraisopropyldisiloxane-1,3-diyl)-N₆-isobutyryl-N₂-[(imidazol-1-yl)propyl]-9-(2′-deoxy- -D-erythro-pentofuranosyl)adenosine. (13)

A solution of 12 (12.0 g, 19.42 mmol) in pyridine (100 mL) was allowed to stir at room temperature with triethylamine (10.1 g, 100 mmol) under argon atmosphere. To this stirred solution was added isobutyryl chloride (6.26 g, 60 mmol) dropwise during a 25 minute period. The reaction mixture was stirred under argon for 10 hours and evaporated to dryness. The residue was partitioned between dichloromethane/water and extracted with dichloromethane (2 ×150 mL). The organic extract was washed with brine (30 mL) and dried over anhydrous MgSO₄. The solvent was removed under reduced pressure and the residue was purified by flash chromatography over silica gel using CH₂Cl₂/acetone as the eluent to give the 13 as a foam: ¹H NMR (Me₂SO-d₆) 1.00 (m, 34 H), 1.92 (m, 2 H, CH₂), 2.42 (m, 1 H, C_{2′}H), 2.92 (m, 2 H, C_{2′}H and Isobutyryl CH), 3.24 (m, 2 H, CH₂) 3.86 (m, 3 H, C_{5′}CH₂ and C_{4′}H), 4.40 (m, 2 H, CH₂), 4.74 (m, 1 H, C_{3′}H), 6.22 (m, 1 H, J_{1′,2′}=6.20 Hz, C_{1′}H), 6.82 (t, 1 H, NH), 6.92 (s, 1 H, ImH), 7.18 (s, 1 H, ImH), 7.60 (s, 1 H, ImH), 8.12 (s, 1 H, C₈H), 10.04 (b s, 1 H, NH). Anal. Calcd for C₃₂H₅₄N₈O₅Si₂: C, 55.94; H, 7.92; N, 16.31. Found: C, 55.89; H, 7.82; N, 16.23.

### Example 75

N₆-3′,5′-Tri-O-isobutyryl-N₂-[imidazol-1-yl(propyl)]--9-(2′deoxy- -D-erythro-pentofuranosyl)adenosine. (14)

The crude product 11 (9.2 g, 24.59 mmol) was coevaporated three times with dry DMF/pyridine (100:50 mL). The above dried residue was dissolved in dry DMF (100 mL) and dry pyridine (100 mL) and cooled to 0° C. To this cold stirred solution was added triethylamine (20.2 g, 200 mmol) followed by isobutyryl chloride (15.9 g, 150 mmol). After the addition of IbCl, the reaction mixture was allowed to stir at room temperature for 12 hours. The reaction mixture was evaporated to dryness. The residue was extracted with dichloromethane (2×200 mL), washed with 5% NaHCO₃ (50 mL) solution, water (50 mL), and brine (50 mL). The organic extract was dried over dry MgSO₄ and the solvent was removed under reduced pressure. The residue was purified by flash column using CH₂Cl₂/acetone (7:3) as the eluent. The pure fractions were collected together and evaporated to give 7.0 g (44%) of 14 as a foam: ¹H NMR (Me₂SO-d₆) 1.00 (m, 18 H, 3 Isobutyryl CH₃), 1.98 (m, 2 H, CH₂), 2.42 (m, 3 H, C_{2′}H and 2 Isobutyryl CH), 2.92 (m, 2 H, C_{2′}H and Isobutyryl CH), 3.24 (m, 2 H, CH₂), 4.04 (m, 2 H, CH₂), 4.22 (m, 3 H, C_{5′}CH₂ and C_{4′}H), 5.42 (m, 1 H C_{3′}H), 6.24 (t, 1 H, J_{1′,2′}=6.20 Hz, C_{1′}H), 7.04 (s, 1 H, ImH), 7.12 (t, 1 H, NH), 7.32 (s, 1 H, ImH), 8.00 (s, 1 H, ImH), 8.12 (s, 1 H, C₈H), 10.14 (b s, 1 H, NH). Anal. Calcd for C₂₈H₄₀N₈O₆: C, 57.52; H, 6.89; N, 19.17. Found: C, 57.49; H, 6.81: N, 19.09.

### Example 76

N₂-Isobutyryl-N₂-[imidazol-1-yl(propyl)]-9- (2′-deoxy- -D-erythro-pentofuranosyl)adenosine. (15)

Method 1: To a stirred solution of 13 (2.6 g, 3.43 mmol) in dry tetrahydrofuran (60 mL) was added tetrabutylammonium flouride (1M solution in THF, 17.15 mL, 17.15 mmol) at room temperature. The reaction mixture was stirred at room temperature for 1 hour and quenched with H⁺ resin. The resin was filtered, and washed with pyridine (20 mL) and methanol (50 mL). The filtrate was evaporated to dryness and the residue on purification over silica column using CH₂Cl₂/MeOH (95:5) gave the title compound in 59% (1 g) yield: ¹H NMR (Me₂SO-d₆) 1.04 (m, 6 H, Isobutyryl CH₃), 1.98 (m, 2 H, CH₂), 2.22 (m, 1 H, Isobutyryl CH), 2.70 (m, 1H, C_{2′}H), 2.98 (m, 1H, C_{2′}H), 3.22 (m, 2 H CH₂), 3.52 (2 m, 2 H, C_{5′}CH₂), 3.82 (m, 1 H, C_{4′}H), 4.04 (m, 2 H, CH₂), 4.38 (m, 1 H, C_{3′}H), 4.92 (b s, 1 H, OH), 5.42 (b s, 1 H, OH) 6.22 (t, 1 H, J_{1′,2′}=6.20 Hz, C_{1′}H), 6.92 (s, 1 H, ImH), 7.06 (t, 1 H, NH), 7.24 (s, 1 H, ImH), 7.74 (s, 1 H, ImH), 8.12 (s, 1 H, C₈H), 10.08 (b s, 1 H, NH). Anal. Calcd for C₂₀H₂₈N₈O₄. H₂O; C, 54.04; H, 6.35; N, 25.21. Found: C, 54.14; H, 6.53; N, 25.06.

Method 2: To an ice cold (0 to -5° C.) solution of 14 (7.4 g. 12.65 mmol) in pyridine:EtOH:H₂O (70:50:10 mL) was added 1 N KOH solution (0° C., 25 mL, 25 mmol) at once. After 10 minutes of stirring, the reaction was quenched with H⁺ resin (pyridinium form) to pH 7. The resin was filtered, and washed with pyridine (25 mL) and methanol (100 mL). The filtrate was evaporated to dryness and the residue was purified by flash chromatography over silica gel using CH₂Cl₂/MeOH (9:1) as eluent. The pure fractions were pooled together and evaporated to give 1.8 g (37%) of 15.

### Example 77

5′-O-(4,4′-Dimethoxytrityl)-N₆-isobutyryl-N₂-[imidazol-1-yl (propyl)]-9-(2′deoxy- -D-erythro-pentofuranosyl)adenosine.

To a well dried (coevaporated three times with dry pyridine before use) solution of 15 (3.6 g, 8.11 mmol) in dry pyridine (100 mL) was added triethylamine (1.01 g, 10.00 mmol) followed by 4,4′-dimethoxytrityl chloride (3.38 g, 10.00 mmol) at room temperature. The reaction mixture was stirred under argon for 10 hours and quenched with methanol (20 mL). After stirring for 10 minutes, the solvent was removed under reduced pressure. The residue was dissolved in dichloromethane (250 mL), washed with water (50 mL), and brine (50 mL), and dried over MgSO₄. The dried organic extract was evaporated to dryness to an orange foam. The foam was purified by flash chromatography over silica gel using CH₂Cl₂/MeOH (95:5) as eluent. The required fractions were collected together and evaporated to give 4.6 g (76%) of 16 as amorphous solid: ¹H NMR (Me₂SO-d₆) 1.04 (m, 6 H, Isobutyryl CH₃), 1.90 (m, 2 H, CH₂), 2.30 (m, 1 H, C_{2′}H), 2.82 (m, 1 H, C_{2′}H), 2.94 (m, 1 H, Isobutyryl CH), 3.14 (m, 4 H, CH₂ and C_{5′}CH₂), 3.72 (m, 6 H, OCH₃), 3.92 (m, 3 H, CH₂ and C_{4′}H), 4.44 (m, 1 H, C_{3′}H), 5.44 (b s, 1 H, C_{5′}OH), 6.28 (t, 1 H, J_{1′,2′}=6.20 Hz, C_{1′}H), 6.72-7.32 (m, 18 H, ImH, NH and ArH), 7.64 (s, 1 H ImH), 8.02 (s, 1 H, C₈H), 10.10 (b s, 1 H, NH). Anal. Calcd for C₄₁H₄₆N₈O₆: C, 65.93; H, 6.21; N, 15.00. Found: C, 65.81; H, 6.26; N, 14.71.

### Example 78

3′-O-[(N,N-diisopropylamino)( -cyanoethoxy)phosphanyl]-5′-O-(4,4′-dimethoxytrityl-N₆-isobutyryl-N₂-[imidazol-1-yl(propyl)]-9-(2′deoxy- -D-erythro-pentofuranosyl)adenosine.

The substrate 16 (4.2 g, 5.6 mmol) was coevaporated with dry pyridine(50 mL) three times. The resulting residue was dissolved in dry dichloromethane (50 mL) and cooled to 0° C. in a ice bath. To this cold stirred solution was added N,N-diisopropylethylamine (1.44 g, 11.2 mmol) followed by ( -cyanoethoxy)chloro (N,N-diisopropylamino)phosphane (1.32 g, 5.6 mmol) over a period of 15 minutes. After the addition, the reaction mixture was stirred at 0° C. for 1 hour and room temperature for 2 hours. The reaction was diluted with dichloromethane (150 mL) and washed with 5% NaHCO₃ solution (25 mL) and brine (25 mL). The organic extract was dried over MgSO₄ and the solvent was removed under reduced pressure. The residue was purified by flash chromatography over silica gel using CH₂Cl₂/MeOH (98:2) containing 1% triethylamine as eluent. The pure fractions were collected together and evaporated to dryness to give 3.9 g (73%) of 17.

### Example 79

N₂-[Imidazol-4-yl(ethyl)]-9-(2′-deoxy- -D-erythro-pentofuranosyl)guanosine.

A mixture of 3 and histamine (4.4 g, 40.00 mmol) in 2-methoxyethanol (60 mL) was heated at 110° C. in a steel bomb for 12 hours. The steel bomb was cooled to 0° C., opened carefully, and the precipitated solid was filtered, washed with acetone and dried. The dried material was recrystallized from DMF/H₂O for analytical purposes. Yield 6 g (79%): mp 220-22° C.: ¹H NMR (Me₂SO-d₆) 2.22 (m, 1 H, C_{2′}H), 2.64 (m, 1 H, C_{2′}H), 2.80 (m, 1 H, CH₂), 3.52 (m, 4 H, CH₂ and C_{5′}CH₂), 3.80 (m, 1 H, C_{4′}H), 4.42 (m, 1 H, C_{3′}H), 4.98 (b s, 1 H, C_{5′}OH), 5.44 (b s, 1 H, C_{3′}OH), 6.16 (t, 1 H, J_{1′,2′}=6.20 Hz, C_{1′}H), 6.44 (b s, 1 H, NH), 6.84 (s, 1 H, ImH), 7.56 (s, 1 H, ImH), 7.92 (s, 1 H, C₈H), 10.60 (b s, 1 H, NH), 11.90 (b s, 1 H, NH). Anal. Calcd for C₁₅H₁₉N₇O₄: C, 49.85; H, 5.30; N, 27.13. Found: C, 49.61; H, 5.21; N, 26.84.

### Example 80

3′,5′-O-(Tetraisopropyldisiloxane-1,3-diyl)-N₂-(imidazol-4-yl(ethyl)-9-(2′-deoxy- -D-erythro-pentofuranosyl)guanosine.

To a stirred suspension of 18 (2.4 g, 6.65 mmol) in dry DMF (50 mL) and dry pyridine (20 mL) was added triethylamine (4.04 g, 40.00 mmol) followed by 1,3-dichloro-1,1,3,3-tetraisopropyldisiloxane (4.18 g, 13.3 mmol) at room temperature. After the addition of TipSiCl, the reaction mixture was stirred overnight and evaporated to dryness. The residue was purified by flash chromatography over silica gel using CH₂Cl₂/MeOH (9:1) as eluent. The pure fractions were pooled together and evaporated to dryness to give 3.2 g (80%) of 19. The pure product was crystallized from acetone/dichloromethane as colorless solid. mp 245-247° C.: ¹H NMR (Me₂SO-d₆) 1.00 (m, 28 H), 2.46 (m, 1 H, C_{2′}H), 2.72 (m, 1 H, C_{2′}H), 2.84 (m, 1 H, CH₂), 3.54 (m, 2 H, CH₂), 3.90 (m, 3 H, C_{4′}H and C_{5′}CH₂), 4.70 (m, 1 H, C_{3′}H), 6.12 (t, 1 H, J_{1′,2′}=6.20 Hz, C_{1′}H), 6.68 (b s, 1 H, NH), 7.20 (s, 1 H, ImH), 7.80 (s, 1 H, ImH), 8.40 (s, 1 H, C₈H) 10.72 (b s, 1 H, NH). Anal. Calcd for C₂₇H₄₅N₇O₅Si₂: C, 53.70; H, 7.51; N, 16.24. Found: C, 53.38; H, 7.63; N, 15.86.

### Example 81

3′5′-O-(Tetraisopropyldisiloxane-1,3-diyl)-6-O-diphenyl-carbamoyl-N₂-[(N₁-diphenylcarbamoyl) imidazol-4-yl(ethyl)]-9-(2′-deoxy- -D-erythro-pentofuranosyl)guanosine. (20)

To a well stirred solution of the substrate 19 (6.03 g, 10.00 mmol) in dry DMF (50 mL) and dry pyridine (50 mL) was added N,N-diisopropylethylamine (5.16 g, 40.00 mmol) followed by diphenylcarbamoyl chloride (6.93 g, 30.00 mmol) at room temperature. The reaction mixture was allowed to stir at room temperature for 5 hours and evaporated to dryness. The residue was dissolved in CH₂Cl₂ (400 mL), washed with water (100 mL) and brine (50 mL), dried over MgSO₄, and evaporated to dryness. The residue was purified by flash chromatography using hexane/acetone (8:2) to give the title compound in 78.5 w (7.8 g) yield: ¹H NMR (Me₂SO-d₆) 1.04 (m,28 H), 2.54 (m, 1 H, C_{2′}H), 2.65 (m, 1 H, C_{2′}H), 2.72 (m, 2 H, CH₂), 3.64 (m, 2 H, CH₂), 3.86 (m, 1 H, C_{4′}H), 4.00 (m, 2 H, C_{5′}CH₂), 4.74 (m, 1 H, C_{3′}H), 5.30 (b s, 1 H, NH), 6.22 (m, 1 H, C_{1′}H), 6.72 (s, 1 H, ImH), 7.12-7.50 (m, 20 H, ArH), 7.70 (s, 1 H, ImH), 7.86 (s, 1 H, C₈H). Anal. Calcd for C₅₃H₆₃N₉O₇Si₂: C, 64.02; H, 6.39; N, 12.68. Found: C, 64.13; H, 6.43; N, 12.79.

### Example 82

6-O-Diphenylcarbamoyl-N₂-[(N₁-diphenylcarbamoyl)imidazol-4-yl(ethyl)]-9-(2′-deoxy- -D-erythro-pentofuranosyl)guanosine. (21)

To a stirred solution of the protected derivative of 20 (1.8 g, 1.81 mmol) in pyridine/THF (30:20 mL) was added a 0.5M tetrabutyl-ammonium fluoride [prepared in a mixture of tetrahydrofuran-pyridine-water (8:1:1;v/v/v; 20 mL)] at room temperature. The reaction mixture was stirred for 15 minutes and quenched with H⁺ resin (pydinium form) to pH 6-7. The resin was filtered off, and washed with pyridine (25 mL) and methanol (30 mL). The filtrate was evaporated to dryness and the residue was purified by flash chromatography using CH₂Cl₂/MeOH (95:5) to give 1.2 g (88%) of 21 as a colorless amorphous solid: ¹H NMR (Me₂SO-d₆) 2.32 (m, 1 H, C_{2′}H), 2.72 (m, 2 H, CH₂), 2.94 (m, 1 H, C_{2′}H), 3.46 (m, 1 H, C_{4′}H), 3.54-3.88 (m, 4 H, CH₂ and C_{5′}CH₂), 4.00 (b s, 1 H, C_{3′}H), 5.20 (b s, 2 H, OH), 5.42 (t, 1 H, NH), 6.10 (t, 1 H, J_{1′,2′}=6.20 Hz C_{1′}H), 6.80 (s, 1 H, ImH), 7.14-7.48 (m, 20 H, ArH), 7.64 (s, 1 H, ImH), 7.74 (s, 1 H, C₈H) . Anal. Calcd for C₄₁H₃₇N₉O₆: C, 65.50; H, 4.96; N, 16.77. Found: C, 65.31; H, 5.10; N, 16.40.

### Example 83

5′-O-(4,4′-Dimethoxytrityl)-6-diphenylcarbamoyl-N₂-[(N₁-diphenylcarbamoyl)imidazol-4-yl (ethyl)]-9- (2′-deoxy--D-erythro-pentofuranosyl)guanosine.

To a well dried solution of the substrate 21 (1.4 g, 1.87 mmol) in dry pyridine (70 mL) was added triethylamine (0.30 g, 3.0 mmol) followed by 4,4′-dimethoxytrityl chloride (0.85 g, 2.5 mmol) at room temperature. The stirring was continued overnight under argon atmosphere. Methanol (10 mL) was added, stirred for 10 minutes and evaporated to dryness. The residue was dissolved in CH₂Cl₂ (150 mL), washed with water (20 mL) and brine (20 mL), dried over MgSO₄, and the solvent removed under reduced pressure. The crude product was purified by flash chromatography over silica gel using CH₂Cl₂/acetone (7:3) containing 1% triethylamine as eluent. Yield 1.4 g (71%): ¹H NMR (Me₂SO-d₆) 2.44 (m, 1 H, C_{2′}H), 2.62 (m, 2 H, CH₂), 2.98 (m, 1 H, C_{2′}H), 3.26 (m, 4 H, CH₂ and C_{5′}CH₂), 3.40 (m, 1 H, C_{4′}H), 3.68 (2 s, 6 H, 2H OCH₃), 4.00 (m, 1 H, C_{3′}H), 5.34 (t, 1 H, NH), 5.44 (b s, 1 H, C_{3′}OH), 6.12 (m, 1 H, C_{1′}H), 6.66-7.48 (m, 34 H, ImH and ArH), 7.62 (s, 1 H, ImH), 7.78 (s, 1 H, C₈H). Anal. Calcd for C₆₂H₅₅N₉O₈₄: C, 70.64; H, 5.26; N, 11.96. Found: C, 70.24; H, 5.39; N, 11.66.

### Example 84

3′-O-[(N,N-Diisopropylamino)(-cyanoethoxy)phosphanyl]-5′-O-(4,4′-dimethoxytrityl)-6-0-diphenylcarbamoyl-N₂-[(N₁-diphenylcarbamoyl) imidazol-4-yl(ethyl)]-9-(2′-deoxy- -D-erythro-pentofuranosyl)guanosine.

Well dried 22 was dissolved in dry dichloromethane (30 mL) and cooled to 0° C. under argon atmosphere. To this cold stirred solution was added N,N-diisopropylethylamine (0.39 g, 3.00 mmol) followed by ( -cyanoethoxy)chloro (N,N-diisopropylamino)phosphane (0.71 g, 3.0 mmol) over a period of 10 minutes. The reaction mixture was allowed to stir at room temperature for 2 hours and diluted with CH₂Cl₂ (120 mL). The organic layer was washed with 5% NaHCO₃ (25 mL), water (25 mL), and brine (25 mL). The extract was dried over anhydrous MgSO₄ and evaporated to dryness. The residue was purified by flash using hexane/ethyl acetate (3:7) containing 1% triethylamine as eluent. The pure fractions were pooled together and concentrated to dryness to give 1.0 g (70%) of 23 as a foam: ¹H NMR (Me₂SO-d₆) 1.12 (m, 12 H, 2 Isobutyryl CH₃), 2.52 (m, 5 H, C₂,H, CH₂ and Isobutyryl CH), 2.62 (m, 2 H), 3.06 (m, 1 H, C_{2′}H), 3.24 (m, 2 H, CH₂) 3.40 (m, 2 H, CH₂), 3.50-3.80 (m, 10 H, 2 OCH₃, CH₂ and C_{5′}CH₂), 4.08 (m, 1 H, C_{4′}H), 4.82 (m, 1 H, C_{3′}H), 5.74 (b s, 1 H, NH), 6.24 (m, 1 H, C_{1′}H), 6.64-7.52 (m, 34 H, ImH and ArH), 7.62 (s, 1 H, ImH), 7.94 (s, 1 H, C₈H)

### Example 85

N₂-Nonyl-9-(2′-deoxy- -D-erythro-pentofuranosyl)guanosine.

A mixture of 2-chloro-2′-deoxyinosine and compound 3 (9.5 g, 33.22 mmol) and nonylamine (9.58 g, 67.00 mmol) in 2-methoxyethanol (60 mL) was heated at 120° C. for 12 hours in a steel bomb. The steel bomb was cooled to 0° C., opened carefully and the solvent removed under reduced pressure. The residue was coevaporated with a mixture of dry pyridine/dry toluene (50 mL each). The above process was repeated for three times and the resultant residue was carried over to the next reaction without further purification. A small amount of material was precipitated from the solution which was filtered and dried: mp 164-167° C.: ¹H NMR (Me₂SO-d₆) 0.82 (t, 3 H, CH₃), 1.24 (m, 12 H, 6 CH₂), 1.48 (m, 2 H, CH₂), 2.18 (m, 1 H, C_{2′}H), 2.62 (m, 1 H, C_{2′}H), 3.22 (m, 2 H, CH₂), 3.50 (m, 2 H, C_{5′}CH₂), 3.78 (m, 1 H, C_{4′}H), 4.32 (m, 1 H, C_{3′}H), 4.84 (t, 1 H, C_{5′}OH), 5.24 (m, 1 H, C_{3′}OH), 6.12 (m, 1 H, C_{1′}H), 6.44 (b s, 1 H, NH), 7.86 (s, 1 H, C₈H), 10.52 (b s, 1 H, NH) . Anal. Calcd for C₁₉H₃₁N₅O₄. H₂O: C, 55.45; H, 8.08; N, 17.00. Found: C, 55.96; H, 7.87; N, 16.59.

### Example 86

N₂,3′,5′-Tri-O-isobutyryl-N₂-nonyl-9-(2′-deoxy--D-erythro-pentofuranosyl)guanosine.

The crude product of 84 (189, 32.91 mmol) was coevaporated three times with a mixture of dry DMF/pyridine (50 mL each). The residue was dissolved in dry pyridine (150 mL) and cooled to 0° C. To this cold stirred solution was added triethylamine (30.3 g, 300 mmol) followed by isobutyryl chloride (21.2 g, 200 mmol) over a 30 minute period. After the addition of IbCl, the reaction mixture was allowed to stir at room temperature for 10 hours and was then evaporated to dryness. The residue was partitioned between CH₂Cl₂/water (300:150 mL) and extracted in CH₂Cl₂. The organic extract was washed with 5% NaHCO₃ (50 mL), water (50 mL) and brine (50 mL), dried over anhydrous MgSO₄, and evaporated to dryness. The residue was purified by flash chromatography over silica gel using CH₂Cl₂/EtOAc (6:4) as eluent. The pure fractions were pooled and evaporated to give 10 g (40%) of 25 as foam: ¹H NMR (Me₂SO-d₆) 0.82 (t, 3 H, CH₃), 1.12 (m, 30 H, 3 Isobutyryl CH₃ and 6 CH₂), 1.44 (m, 2 H, CH₂), 2.54 (m, 4 H, C_{2′}H and 3 Isobutyryl CH), 3.00 (m, 1 H, C_{2′}H), 3.62 (m, 2 H, CH₂), 4.20 (m, 3 H, C_{5′}CH₂ and C_{4′}H), 5.32 (m, 1 H, C_{3′}H), 6.24 (t, 1 H, J_{1′,2′}=6.20 Hz, C_{1′}H), 8.28 (s, 1 H, C₈H), 12.82 (b s, 1 H, NH). Anal. Calcd for C₃₁H₄₉N₅O₇: C, 61.67; H, 8.18; N, 11.60. Found: C, 61.59; H, 8.23; N, 11.34.

### Example 87

3′,5′-O-(Tetraisopropyldisiloxane-1,3-diyl)-N₂-nonyl-9-(2′-deoxy- -D-erythro-pentofuranosyl)guanosine.

To a well dried solution of the crude product of 85 (16.4 g, 30.00 mmol) in dry DMF (100 mL) and dry pyridine (100 mL) was added triethylamine (10.1 g, 100 mmol) and 1,3-dichloro-1,1,3,3-tetraisopropyldisiloxane (15.75 g, 50 mmol) during 30 min period. The reaction mixture was allowed to stir at room temperature overnight and was then evaporated to dryness. The crude product was dissolved in CH₂Cl₂ (300 mL), washed with water (100 mL), and brine (50 mL). The extract was dried over MgSO₄ and the solvent was removed under reduced pressure. The residue was purified over silica column using CH₂Cl₂/acetone (7:3) to give 14 g (59%) of 26 as colorless foam. This on crystallization with the same solvent provided crystalline solid. mp 210-212° C.: ¹H NMR (Me₂SO-d₆) 0.82 (m, 3 H, CH3), 1.02 (m, 28 H), 1.24 (m, 12 H, 6 CH₂), 1.50 (m, 2 H, CH₂), 2.42 (m, 1 H, C_{2′}H) 2.84 (m, 1 H, C_{2′}H), 3.24 (m, 2 H, CH₂), 3.82 (m, 2 H, C_{5′}CH₂), 3.92 (m, 1 H, C_{4′}H), 4.72 (m, 1 H, C_{3′}H), 6.12 (t, 1 H, J_{1′,2′}=6.20 Hz, C_{1′}H), 6.36 (b s, 1 H, NH), 7.78 (s, 1 H, C₈H), 10.38 (b s, 1 H, NH). Anal. Calcd for C₃₁H₅₇N₅O₅Si₂: C, 58.54; H, 9.03; N, 11.01. Found: C, 58.64; H, 9.09; N, 10.89.

### Example 88

N₂-Isobutyryl-3′,5′-O-(tetraisopropyldisiloxane-1,3-diyl)-N₂-nonyl-9-(2′-deoxy- -D-erythro-pentofuranosyl)guanosine.

To a solution of 86 (14.0 g, 17.72 mmol) in dry DMF (50 mL) and dry pyridine (150 mL) was added triethylamine (3.54 g, 35.00 mmol) and isobutyryl chloride (3.71 g, 3.5 mmol). The reaction mixture was stirred at room temperature overnight and evaporated to dryness. The residue was dissolved in CH₂Cl₂ (250 mL), washed with 5% NaHCO₃ (50 mL), water (50 mL) and brine (50 mL), dried over MgSO₄, and the solvent removed under reduced pressure. The residue was purified by flash chromatography over silica gel using CH₂Cl₂/acetone (9:1) as eluent. The pure fractions were pooled together and evaporated to dryness to give 12.0 g (77%) of the title compound as foam: ¹H NMR (Me₂SO-d6) 0.80 (m, 3 H, CH₃), 0.98 (m, 34 H), 1.20 (m, 12 H, 6 CH₂), 1.42 (m, 2 H, CH₂), 2.52 (m, 2 H, C_{2′}H and Isobutyryl CH), 2.82 (m, 1 H, C_{2′}H), 3.62 (m, 2 H, CH₂), 3.84 (m, 3 H, C_{5′}CH2 and C_{4′}H), 4.72 (m, 1 H, C_{3′}H), 6.22 (t, 1 H, J_{1′,2′}=6.20 Hz, C1′H), 8.18 (s, 1 H, C₈H), 12.80 (b s, 1 H, NH).

### Example 89

N₂-Isobutyryl-N₂-nonyl-9-(2′-deoxy- -D-erythro-pentofuranosyl)guanosine. (28)

Method 1: The substrate of 85 (5.00 g, 6.6 mmol) was dissolved in methanol (100 mL) and treated with concentrated NH₄OH (100 mL). The reaction mixture was stirred for 4 hours at room temperature and evaporated to dryness. The residue was purified by flash chromatography over silica gel using CH₂Cl₂/MeOH (95:5) as eluent. The required fractions were collected together and evaporated to dryness and the residue on crystallization from CH₂Cl₂/acetone gave a colorless crystalline solid. yield 2 g (66%): mp 113-115° C.

Method 2: A stirred solution of 27 (4.29 g, 4.99 mmol) in dry tetrahydrofuran (50 mL) was treated with 1M solution of tetrabutylammonium fluoride (20 mL, 20.00 mmol). The reaction mixture was stirred at room temperature for 4 hours and evaporated to dryness. The residue was purified by flash chromatography using CH₂Cl₂/MeOH (95:5) to give 1.59 g (69%) of 28: ¹H NMR (Me₂SO-d₆) 0.80 (m, 3 H, CH₃), 0.98 (m, 6 H, Isobutyryl CH₃), 1.16 (m, 12 H, 6 CH₂), 1.42 (m, 2 H, CH₂), 2.24 (m, 1 H, C_{2′}H), 2.52 (m, 2 H, C_{2′}H and Isobutyryl CH), 3 .50 (m, 2 H, C_{5′}CH₂), 3.62 (m, 2 H, CH₂), 3.82 (m, 1 H, C_{4′}H), 4.36 (m, 1 H, C_{3′}H), 4.94 (t, 1 H, C_{5′}OH), 5.34 (m, 1 H, C_{3′}OH), 6.22 (t, 1 H, J_{1′,2′}=6.20 Hz, C_{1′}H), 8.28 (s, 1 H, C₈H), 12.78 (b s, 1 H, NH) . Anal. Calcd for C₂₃H₃₇N₅O₅: C, 59.59; H, 8.05; N, 15.11. Found: C, 59.50; H, 8.08; N, 15.06.

### Example 90

5′-O-(4,4′-Dimethoxytrityl)-N₂-isobutyryl-N₂-nonyl-9-(2′-deoxy- -D-erythro-pentofuranosyl)guanosine. (29)

To a stirred solution of 28 (2.00 g, 4.32 mmol) in dry pyridine (75 mL) was added triethylamine (0.61 g, 6.00 mmol) and 4,4′-dimethoxytrityl chloride (2.03 g, 6.00 mmol) at room temperature. The reaction was stirred under argon atmosphere for 6 hours and quenched with methanol (10 mL). The solvent was removed under reduced pressure and the residue dissolved in CH₂Cl₂ (150 mL). The organic extract was washed with water (25 mL) and brine (25 mL), dried over MgSO₄, and evaporated to dryness. The residue was purified by flash chromatography over silica gel using CH₂Cl₂/acetone (7:3) as eluent. The pure fractions were pooled together and evaporated to give 2 g (60%) of 29 as foam: ¹H NMR (Me₂SO-d₆) 0.80 (m, 3 H, CH₃), 0.96 (m, 6 H, Isobutyryl CH₃), 1.16 (m, 12 H, 6 CH₂), 1.36 (m, 2 H, CH₂), 2.32 (m, 1 H, C_{2′}H), 2.60 (m, 1 H, Isobutyryl CH), 2.72 (m, 1 H, C_{2′}H), 3.12 (m, 2 H, CH₂), 3.52 (m, 2 H, C_{5′}CH₂), 3.70 (2 d, 6 H, 2 OCH₃), 3.90 (m, 1 H, C_{4′}H), 4.34 (m, 1 H, C_{3′}H), 5.36 (m, 1 H, C_{3′}OH), 6.26 (t, 1 H, J_{1′,2′}=6.20 Hz, C_{1′}H), 6.70-7.36 (m, 13 H, ArH), 8.18 (s, 1 H, C₈H). Anal. Calcd for C₄₄H₅₆N₅O₇: C, 68.90; H, 7.36; N, 9.31. Found: C, 68.76; H, 7.47; N, 9.09.

### Example 91

3′-O-[(N,N-Diisopropylamino)( -cyanoethoxy)phosphanyl]-5′-O-(4,4′-dimethoxytrityl)-N₂-isobutyryl-N₂-nonyl-9-(2′-deoxy- -D-erythro-pentofuranosyl)guanosine. (30)

A well dried solution of 29 (1.7 g, 2.22 mmol) in dry dichloromethane (30 mL) was cooled to 0° C. To this cold solution was added N,N-diisopropyethylamine (0.57 g, 4.4 mmol) and ( -cyanoethoxy)chloro(N,N-diisopropylamino)phosphane (0.94 g, 4.0 mmol) under argon atmosphere. The reaction mixture was stirred at room temperature for 2 hours and diluted with CH₂Cl₂ (170 mL). The organic extract was washed with 5% NaHCO₃ (25 mL), water (25 mL) and brine (25 mL), dried over Na₂SO₄, and evaporated to dryness. The residue was purified on a silica column using CH₂Cl₂/acetone (9:1) containing 1% triethylamine as eluent. The pure fractions were pooled together and evaporated to dryness to give 1.5 g (53%) of 30.

### Example 92

3′,5′-O-(Tetraisopropyldisiloxane-1,3-diyl)-2-chloro-9-(2′-deoxy- -D-erythro-pentofuranosyl)adenosine. (31)

Compound 31 was prepared from compound 10 by following the procedure used for the preparation of 12. Starting materials used: 10 (4.30 g, 15.09 mmol), 1,3-dichloro-1,1,3,3-tetraisopropyldisiloxane (4.74 g, 15.1 mmol), dry TEA (3.05 g, 30.2 mmol), and dry pyridine (100 mL). The crude product was purified by flash chromatography using CH₂Cl₂/acetone (7:3) as eluent to give 7.3 g (92%) of 31. The pure product was crystallized from ethylacetate/hexane as a colorless solid. mp 183-185° C.: ¹H NMR (Me₂SO-d₆) 1.00 (m, 28 H), 2.54 (m, 1 H, C_{2′}H), 2.82 (m, 1 H, C_{2′}H), 3.76 (m, 1 H, C_{4′}H), 3.86 (m, 2 H, C_{5′}CH₂), 5.08 (m, 1 H, C_{3′}H), 6.22 (t, 1 H, J_{1′,2′}=6.20 Hz, C_{1′}H) 7.82 (b s, 2 H, NH₂), 8.22 (s, 1 H, C₈H. Anal. Calcd for C₂₂H₃₈ClN₅O₄Si₂: C, 50.02; H, 7.25; N, 13.26, Cl, 6.72. Found: C, 50.24; H, 7.28; N, 13.07, Cl, 6.63.

### Example 93

3′,5′-O-(Tetraisopropyldisiloxane-1,3-diyl)-2-chloro-N₆-benzoyl-9-(2′-deoxy- -D-erythro-pentofuranosyl)adenosine. (32)

A well dried solution of 31 (8 g, 15.00 mmol) in dry pyridine (150 mL) was allowed to react with triethylamine (4.55 g, 45.00 mmol) and benzoyl chloride (6.3 g, 45.00 mmol) at room temperature for 12 hours under argon atmosphere. The reaction mixture was evaporated to dryness. The residue was partitioned between CH₂Cl₂/water and extracted in CH₂Cl₂ (2×150 mL). The organic extract was washed with brine (60 mL), dried over MgSO₄ and evaporated to dryness. The residue was purified and silica column using CH₂Cl₂/acetone as eluent and crystallization from the same solvent gave 8.2 g (86%) of 32. mp 167-170° C.: ¹H NMR (Me₂SO-d6) 1.00 (m, 28 H), 2.60 (m, 1 H, C_{2′}H), 3.02 (m, 1 H, C_{2′}H), 3.84 (m, 3 H, C_{5′}CH₂ and C_{4′}H), 5.04 (m, 1 H, C_{3′}H), 6.34 (d, 1 H, C_{1′}H), 7.42-7.84 (m, 5 H, ArH), 8.70 (s, 1 H, C₈H). Anal. Calcd for C₂₉H₄₂ClN₅O₅Si₂: C, 55.08; H, 6.69; N, 11.08, Cl, 5.61. Found: C, 55.21; H, 6.79; N, 11.19, Cl, 5.70.

### Example 94

N₆-Benzoyl-2-chloro-9-(2′-deoxy- -D-erythro-pentofuranosyl) adenosine. (33)

To a stirred solution of 32 (7.9 g, 12.5 mmol) in dry THF (100 mL) was added 1M solution of tetrabutylammonium fluoride (50 mL, 50.00 mmol) slowly over a 15 minute period at room temperature. The reaction mixture was stirred for 6 hours and evaporated to dryness. The residue was purified by flash chromatography using CH₂Cl₂/acetone (7:3) as eluent to give 3.88 g (80%) of 33. mp≧275° C. dec: ¹H NMR (Me₂SO-d₆) 2.34 (m, 1 H, C_{2′}H), 2.72 (m, 1 H, C_{2′}H), 3.58 (m, 2 H, C_{5′}CH₂), 3.88 (m, 1 H, C_{4′}H), 4.42 (m, 1 H, C_{3′}H) 4.96 (t, 1H, C_{5′}OH), 5.38 (d, 1 H, C_{3′}OH), 6.40 (t, 1 H, J_{1′,2′}=6.20 Hz, C_{1′}H), 7.52 (m, 2 H, ArH), 7.64 (m, 1 H, ArH), 8.04 (d, 2 H, ArH), 8.70 (s, 1 H, C₈H), 11.52 (b s, 1 H, NH). Anal. Calcd for C₁₇H₁₆ClN₅O₄: C, 52.37; H, 4.14; N, 17.97; Cl, 9.11. Found: C, 52.31; H, 4.07; N, 17.94; Cl, 9.03.

### Example 95

5′-O-(4,4′-Dimethoxytrityl)-N₆-benzoyl-2-chloro-9-(2′-deoxy- -D-erythro-pentofuranosyl)adenosine. (34)

The compound was prepared from 33 by following the procedure used for the preparation of 8. Starting materials used: 33 (2.5 g. 6.43 mmol), 4,4′-dimethoxytrityl chloride (2.37 g, 7.0 mmol), dry TEA (0.71 g, 7.0 mmol) and dry pyridine (100 mL). The crude product was purified by flash chromatography using CH₂Cl₂/EtOAc (7:3) containing 1% triethylamine as the eluent to give 3 g (68%) of 34 as foam: ¹H NMR (Me₂SO-d₆) 2.34 (m, 1 H, C_{2′}H), 2.82 (m, 1 H, C_{2′}H) 3.18 (m, 2 H, C_{5′}CH₂), 3.64 (2d, 6 H, OCH₃), 3.98 (m, 1 H, C_{4′}H), 4.44 (m, 1 H, C_{3′}H), 5.40 (d, 1 H, OH), 6.42 (t, 1 H, J_{1′,2′}=6.20 Hz, C_{1′}H), 6.74 (m, 4 H, ArH), 7.16 (m, 7 H, ArH), 7.32 (m, 2 H, ArH), 7.52 (m, 7 H, ArH), 7.64 (m, 1 H, ArH), 8.04 (m, 2 H, ArH), 8.58 (s, 1 H, C₈H), 11.50 (b s, 1 H, NH). Anal. Calcd for C₃₈H₃₄ClN₅O₆: C, 65.93; H, 4.95; N, 10.12; Cl, 5.13. Found: C, 65.55; H, 5.16; N, 9.73; Cl, 5.10.

### Example 96

3′-O-[(N,N-Diisopropylamino)( -cyanoethoxy)phosphanyl]-5′-O-(4,4′-dimethoxytrityl)-N₆-benzoyl-2-chloro-9-(2′-deoxy- -D-erythro-pentofuranosyl)adenosine. (35)

The title compound was prepared from 34 by following the procedure used for the preparation of 9. Starting materials used: Compound 34 (2.4 g, 3.47 mmol), N, N-diisopropylethylamine (1.22 mL, 7.00 mmol), ( -cyanoethoxy) chloro(N,N-diisopropylamino)phosphene (1.65 g, 7.00 mmol) and dry CH₂Cl₂ (30 mL). The crude product was purified by flash chromatography using hexane-ethyl acetate (1:1) containing 1% triethylamine as eluent. The pure fractions were pooled together and evaporated to dryness to give 1.8 g (58%) of 35. The foam was dissolved in dry dichloromethane (10 mL) and added dropwise into a well stirred hexane (1500 mL) under argon atmosphere. After the addition, stirring was continued for additional 1 hour and the precipitated solid was filtered, washed with hexane and dried over solid NaOH for 3 hours. The dried powder showed no traces of impurity in ³¹p spectrum: ¹H NMR (Me₂SO-d₆) 1.18 (m, 12 H, Isobutyryl CH₃), 2.58 (m, 3 H, C_{2′}H and Isobutyryl CH), 2.98 (m, 1 H, C_{2′}H), 3.34 (d, 2 H, CH₂), 3.64 (m, 2 H, C_{5′}CH₂), 3.72 (m, 8 H, 2 OCH3 and CH₂), 4.24 (m, 1 H, C_{4′}H), 4.82 (m, 1 H, C_{3′}H), 6.36 (t, 1 H, J_{1′,2′}=6.20 Hz, C_{1′}H), 6.76 (m, 4 H, ArH), 7.22 (m, 7 H, ArH), 7.38 (m, 2 H, ArH), 7.52 (m, 2 H, ArH), 7.64 (m, 1 H, ArH), 7.98 (m, 2 H, ArH), 8.24 (s, 1 H, C₈H), 9.34 (b s, 1 H, NH).

### Example 97

3′,5′-O-(Tetraisopropyldisiloxane-1,3-diyl)-N₂-ethyl-9-(2′-deoxy- -D-erythro-pentofuranosyl)guanosine. (36)

A solution of 3′,5′-O-(tetraisopropyldisiloxane-1,3-diyl)-2-chloro-9-(2′-deoxy- -D-erythro-pentofuranosyl)-inosine (5.0 g, 9.45 mmol) in 2-methoxyethanol (30 mL) was placed in a steel bomb and cooled to 0° C. Freshly condensed ethylamine (7.0 mL) was quickly added. The steel bomb was sealed and the reaction mixture was stirred at 90° C. for 16 hours. The vessel was cooled and opened carefully. The precipitated white solid was filtered and crystallized from methanol. The filtrate on evaporation gave solid which was also crystallized from methanol. Total yield 3. g (65%). mp≧250° C. dec: ¹H NMR (Me₂SO-d₆) 1.06 (m, 31 H), 2.32 (m, 1 H, C_{2′}H), 2.84 (m, 1 H, C_{2′}H), 3.26 (m, 2 H, CH₂), 4.12 (m, 2 H, C_{5′}CH₂), 4.22 (m, 1 H, C_{4′}H), 4.70 (m, 1 H, C_{3′}H), 6.23 (t, 1 H, J_{1′,2′}=6.20 Hz, C_{1′}H), 6.42 (m, 1 H, NH), 7.87 (s, 1 H, C₈H), 10.58 (b s, 1 H, NH). Anal. Calcd for C₂₄H₄₃N₅O₅Si₂. C, 53.59; H, 8.06; N, 13.02. Found: C, 53.44; H, 8.24; N, 12.91.

### Example 98

3′,5′-O-(Tetraisopropyldisiloxane-1,3-diyl)-6-O-diphenyl-carbamoyl-N₂-ethyl-9-(2′-deoxy- -D-erythro-pentofuranosyl) guanosine. (37)

Compound 36 (2.40 g, 4.46 mmol) was dissolved in anhydrous pyridine (30 mL) at room temperature. To this solution was added N,N-diisoproylethylamine (1.60 mL, 8.93 mmol) followed by diphenylcarbamoyl chloride (2.07 g, 8.93 mmol). The mixture was stirred at room temperature under argon atmosphere for 10 hours. A dark red solution was obtained, which was evaporated to dryness. The residue was purified by flash chromatography on a silica column using CH₂Cl₂/EtoAc as eluent. The pure fractions were collected together and evaporated to give a brownish foam (3.25 g, 99%). ¹H NMR (Me₂SO-d₆) 1.14 (t, 31 H), 2.52 (m, 1 H, C_{2′}H), 3.04 (m, 1 H, C_{2′}H), 3.34 (m, 2 H, CH₂), 3.87 (m, 3 H, C_{5′}CH₂ & C_{4′}H), 4.83 (m, 1 H, C_{3′}H), 6.23 (m, 1 H, C_{1′}H) 7.36 (m, 11 H, ArH & NH), 8.17 (s, 1 H, C₈H). Anal. Calcd for C₃₇H₅₂N₆O₆Si₂. C, 60.71; H, 7.16; N, 11.48. Found: C, 60.33; H, 7.18; N, 11.21.

### Example 99

6-O-Diphenylcarbamoyl-N₂-ethyl-9-(2′-deoxy- -D-erythro-pentofuranosyl)guanosine. (38)

To a stirred solution of 37 (3.25 g, 4.47 mmol) in pyridine (25 mL) was added 0.5 M solution of tetrabutylammonium fluoride (prepared in pyridine/THF/water, 4/1/1,36 mL, 17.88 mmol) at once. The reaction was allowed to stir for 10 minutes and quenched with H⁺ resin (amberlite IRC 50) to pH 7. The resin was filtered and washed with pyridine (20 mL) and MeOH (20 mL). The filtrate was evaporated to dryness. The residue was purified using flash chromatography over a silica column using methylene chloride-acetone as eluent to give 1.84 g (84%) of the pure product as foam. ¹H NMR (Me₂SO-d₆) 1.14 (t, 3 H, CH₂CH₃), 2.22 (m, 1 H, C_{2′}H), 2.76 (m, 1 H, C_{2′}H), 3.34 (m, 2 H, CH₂), 3.57 (m, 2 H, C_{5′}CH₂), 3.84 (m, 1 H, C_{4′}H), 4.42 (m, 1 H, C_{3′}H), 4.91 (t, 1 H, C_{5′}OH), 5.32 (d, 1 H, C_{3′}OH), 6.27 (t, 1 H, J_{1′,2′}=6.20 Hz C1′H), 7.29 (m, 1 H, NH), 7.46 (m, 10 H, ArH), 8.27 (s, 1 H, C₈H). Anal. Calcd for C₂₅H₂₆N₆O₅-·¾H₂O. C, 59.61; H, 5.35; N, 16.68. Found: C, 59.83; H, 5.48; N, 16.21.

### Example 100

N₂-Ethyl-9-(2′-deoxy- -D-erythro-pentofuranosyl)guanosine. (39)

The intermediate of 38 (0.25 g, 0.51 mmol) was stirred in methanolic/ammonia (saturated at 0° C.) in a steel bomb at room temperature for 40 hours. The vessel was cooled to 0° C., opened carefully, and the solvent evaporated to dryness. The solid obtained was crystallized from methanol to give a white powder (0.95 g, 63%): mp 234-238° C. ¹H NMR (Me₂SO-d₆) 1.14 (t, 3 H, CH₂CH₃), 2.18 (m, 1 H, C_{2′}H), 2.67 (m, 1 H, C_{2′}H), 3.34 (m, 2 H, CH₂), 3.52 (m, 2 H, C_{5′}CH₂), 3.82 (m, 1 H, C_{4′}H), 4.36 (m, 1 H, C_{3′}H), 4.89 (t, 1 H, C_{5′}OH), 5.30 (d, 1 H, C_{3′}OH), 6.16 (t, 1 H, J_{1′,2′}=6.20 Hz C_{1′}H), 6.44 (m, 1 H, NH), 7.91 (s, 1 H, C₈H), 10.58 (b s, 1 H, NH).

### Example 101

5′-O-(4,4′-Dimethoxytrityl)-6-O-diphenylcarbamoyl-N₂-ethyl-9-(2′-deoxy- -D-erythro-pentofuranosyl)guanosine. (40)

Compound 38 (1.6 g, 3.26 mmol) was dried well by coevaporation with dry pyridine (3×50 mL). The dried material was dissolved in anhydrous pyridine (25 mL) and allowed to stir under argon atmosphere. To this stirred solution was added triethylamine (0.59 mL, 4.24 mmol) followed by DMTCl (1.44 g, 4.24 mmol). The reaction mixture was stirred at room temperature for 14 hours and quenched with methanol (10 mL). After stirring for 15 minutes, the solvent was removed and the residue was dissolved in methylene chloride (150 mL). The organic extract was washed with saturated NaHCO₃ solution (30 mL), water (30 mL), and brine (30 mL). The methylene chloride extract was dried and evaporated to dryness. The residue was purified by flash chromatography over silica gel using methylene chloride/acetone as eluent. The pure fractions were collected together and evaporated to give a foam (2.24 g, 87%). ¹H NMR (Me₂SO-d₆) 1.10 (t, 3 H, CH₂CH₃), 2.32 (m, 1 H, C_{2′}H), 2.82 (m, 1 H, C_{2′}H), 3.15 (m, 2 H, CH₂), 3.34 (s, 6 H, 2 OCH₃), 3.67 (m, 2 H, C_{5′}CH₂), 3.96 (m, 1 H, C_{4′}H), 4.42 (m, 1 H, C_{3′}H), 5.36 (d, 1 H, C_{3′}OH), 6.30 (t, 1 H, J_{′,2′}=6.20 Hz, C_{1′}H), 6.83 (m, 4 H, ArH), 7.23 (m, 10 H, ArH & NH), 8.17 (s, 1 H, C₈H). Anal Calcd for C₄₅H₄₄N₆O₇. ¼ CH₃OH. ¼ H₂O. C, 68.50; H, 5.78; N, 10.60. Found: C, 68.72; H, 5.42; N, 10.40.

### Example 102

3′-O-[(N,N-Diisopropylamino)( -cyanoethoxy)phosphanyl]-5′-O-(4,4′-dimethoxytrityl)-6-O-diphenylcarbamoyl-N₂-ethyl-9-(2′-deoxy- -D-erythro-pentofuranosyl)guanosine. (41)

The DMT derivative of 40 was dried well overnight at vacuum and dissolved in dry methylene chloride (25 mL). The solution was cooled to 0° C. under argon atmosphere. To this cold stirring solution N,N-diisopropylamine tetrazolide salt (0.24 g, 1.41 mmol) followed by phosphorylating reagent (1.71 mL, 5.66 mmol) were added. The mixture was stirred at room temperature for 12 hours under argon. The solution was diluted with additional methylene chloride (100 mL) and washed with saturated NaHCO₃ solution (50 mL), water (50 mL), and brine (50 mL). The organic extract was dried and evaporated to dryness. The crude product was purified by flash column over silica gel using methylene chloride/ethyl acetate containing 1% triethylamine as eluent. The pure fractions were pooled and evaporated to give 2.5 g (91%) of 41.

### Example 103

N₂-3′,5′-Tri-O-acetyl-9-(2′-deoxy- -D-erythro-pento-furanosyl)guanosine. (42)

Deoxyguanosine (26.10 g, 96.77 mmol) was coevaporated with dry pyridine/DMF (50 mL each) three times. The residue was suspended in dry DMF (50 mL) and dry pyridine (50 mL) at room temperature. To this stirring mixture was added N,N-dimethylaminopyridine (1.18 g, 9.67 mmol) followed by acetic anhydride (109.6 mL, 116 mmol) slowly keeping the temperature below 35° C. After the addition of Ac₂O, the reaction was placed at 80° C. for 4 hours under argon. It was cooled to room temperature and neutralized with iN NaCO₃ solution. The mixture was extracted in CH₂Cl₂ (2×250mL). The organic extract was washed with water (50 mL) and brine (50 mL), dried, and evaporated to dryness. The residue was crystallized from MeOH to give 29.1 g (76%): mp 217-219° C. ¹H NMR (Me₂SO-d6) 2.04 (s, 3 H, COCH₃), 2.09 (s, 3 H, COCH₃), 2.19 (s, 3 H, COCH₃), 2.60 (m, 1 H, C_{2′}H), 3.02 (m, 1 H, C_{2′}H), 4.19 (m, 3 H, C_{4′}H & C_{5′}CH₂), 5.31 (m, 1 H, C_{3′}H), 6.21 (t, 1 H, J_{1′,2′}=6.00 Hz, C_{1′}H), 8.27 (s, 1 H, C₈H), 11.72 (b s, 1 H, NH), 12.02 (b s, 1 H, NH).

### Example 104

6-O-Benzyl-9-(2′-deoxy- -D-erythro-pentofuranosyl)guanosine. (43)

N₂,3′,5′-Tri-O-acetyldeoxyguanosine 42 (1.18 g, 3 mmol) was suspended in dry dioxane (50 mL) under argon atmosphere. To this stirred suspension was added dry benzyl alcohol (0.81 g, 7.5 mmol) followed by triphenyl phosphine (1.96 g, 7.5 mmol). After stirring for 15 minutes, diethylazodicarboxylate (1.30 g, 7.5 mmol) was added dropwise over a 15 minute period at room temperature. The reaction mixture was stirred under argon overnight at room temperature. The solvent was removed and the residue treated with 0.1M sodium methoxide (75 mL) and stirred at room temperature overnight. Glacial acetic acid (0.45 mL) was added, the solvents were evaporated and the residue was partitioned between water and ethyl acetate. The ethyl acetate extracts were dried, evaporated and the residue was chromatographed over silica gel using CH₂Cl2-MeOH mixture. The product (0.5 g, 75%) was obtained as an amorphous white solid after trituration with ether. ¹H NMR (Me₂SO-d₆) 2.22 (m, 1 H, C_{2′}H), 2.60 (m, 1 H, C_{2′}H), 3.56 (m, 2 H, C_{5′}CH₂), 3.80 (m, 1 H, C_{4′}H), 4.37 (m, 1 H, C_{3′}H), 5.01 (t, 1 H, C_{5′}OH), 5.29 (b s, 1 H, C_{3′}OH), 5.52 (s, 2 H, ArCH₂), 6.23 (t, 1 H, J_{1′,2′}=6.66 Hz, C_{1′}H), 6.52 (b s, 2 H, NH₂), 7.40 (m, 2 H, ArH), 7.50 (m, 2 H, ArH), 8.11 (s, 1 H, C₈H) Anal. Calcd for C₁₇H₁₉N₅O₄. C, 57.13; H, 5.36; N, 19.59. Found: C, 57.09; H, 5.42; N, 19.61.

### Example 105

6-O-Benzyl-2-fluoro-9-(2′-deoxy- -D-erythro-pentofuranosyl) purine. (44)

To a stirred suspension of the substrate 43 (5.0 g, 14 mmol) in dry pyridine (20 ml) at -40° C. was added HF/pyridine (Aldrich 18,422-5 70%) in two portions (2×10 mL) under argon atmosphere. After the addition of HF/pyridine, the mixture was warmed up to −10° C., during that time all the solid had gone into solution. Tert-butyl nitrite (4.0 mL) was added slowly during the course of 10 minutes maintaining the temperature between −20° C. and −10° C. At intervals the reaction mixture was removed from the cooling bath and swirled vigorously to ensure thorough mixing. After complete conversion of the starting material (checked by TLC at 15 minute intervals), the reaction mixture was poured onto a vigorously stirred ice cold alkaline solution (70 g of K₂CO₃ in 150 mL of water). The gummy suspension was extracted with methylene chloride (2×200 mL). The organic extract was washed with brine (100 mL), dried and evaporated to dryness. The residue was purified by flash chromatography over silica gel using CH₂Cl₂ MeOH as eluent. The pure fractions were combined and evaporated to give 4.0 g (79%) of 44 as foam. A small quantity was crystallized from methanol as orange crystals. mp: 165-167° C. ¹H NMR (Me₂SO-d6) 2.36 (m, 1 H, C_{2′}H), 2.66 (m, 1 H, C_{2′}H), 3.60 (m, 2 H, C_{5′}CH₂), 3.87 (m, 1 H, C_{4′}H), 4.42 (m, 1 H, C_{3′}H), 4.95 (t, 1 H, C_{5′}OH), 5.36 (d, 1 H, C_{3′}OH), 5.62 (s, 2 H, ArCH₂), 6.34 (t, 1 H, J_{1′,2′}=6.67 Hz, C_{1′}H), 6.46 (m, 4 H, ArH), 8.61 (s, 1 H, C₈H). Anal. Calcd for C₁₇H₁₇FN₄O₄. C, 56.66; H, 4.76; N, 15.55. Found: C, 56.62; H, 4.69; N, 15.50.

### Example 106

5′-O-(4,4′-Dimethoxytrityl)-2-fluoro-9-(2′-deoxy- -D-erythro-pentofuranosyl)inosine. (45)

Compound 44 (5.00 g, 13.89 mmol) was dissolved in methanol (100 mL) and placed in a parr bottle. To this solution Pd/C (5%, 1.00 g) was added and hydrogenated at 45 psi for 2 hours. The suspension was filtered, washed with methanol (50 mL) and the combined filtrate evaporated to dryness. The residue was dissolved in dry pyridine (50 mL) and evaporated to dryness. This was repeated three times and the resulting residue (weighed 4.00 g) was dissolved in dry pyridine (100 mL) under argon atmosphere. To this stirred solution was added triethylamine (1.52 g, 15.0 mmol) and 4,4′-dimethoxytrityl chloride (5.07 g, 15.0 mmol) at room temperature. The reaction mixture was allowed to stir at room temperature under argon atmosphere overnight. It was quenched with methanol (20 mL) and evaporated to dryness. The residue was dissolved in methylene chloride (200 ml) and washed with 5% NaHCO₃ solution (50 mL), water (50 mL), and brine (50 mL). The organic extract was dried, and evaporated to dryness. The residue was suspended in dichlormethane and the insoluble solid filtered. The filtrate was purified by flash chromatography over silica gel using CH₂Cl₂ MeOH as the eluent. The pure fractions were collected and evaporated to give 7.0 g (88%) of the title compound. The insoluble solid was found to be the DMT derivative. mp>220° C. dec: ¹H NMR (Me₂SO-d₆) 2.22 (m, 1 H, C_{2′}H), 2.70 (m, 1 H, C_{2′}H), 3.16 (m, 2 H, C_{5′}CH₂), 3.90 (m, 1 H, C_{4′}H), 4.38 (m, 1 H, C_{3′}H), 5.32 (d, 1 H, C_{3′}OH), 6.16 (t, 1 H, J_{1′,2′}=6.20 Hz, C_{1′}H), 6.82 (m, 4 H, ArH), 7.25 (m, 9 H, ArH), 7.79 (s, 1 H, C₈H).

### Example 107

3′-O-[(N,N-Diisopropylamino)( -cyanoethoxy)phosphanyl]-5′-O-(4,4′-dimethoxytrityl)-2-fluoro-9-(2′-deoxy- -D-erythro-pentofuranosyl)inosine. (46)

The title compound was prepared from 45 by following the procedure used for the preparation of 9. Starting materials used: 45 (7.0 g, 12.24 mmol), N,N-diisopropylethylamine (5.2 mL, 30.00 mmol), ( -cyanoethoxy) chloro(N,N-diisopropylamino)phosphane (5.9 g, 25.00 mmol) and dry CH₂Cl₂ (100 mL). The crude product was purified by flash chromatography using dichloromethane/methanol (95:5) containing 1% triethylamine as eluent. The pure fractions were pooled together and evaporated to dryness to give 7.00 g (75.5%) of 46. The foam was dissolved in dry dichloromethane (30 mL) and added dropwise into a well stirred hexane (2500 ml) under argon atmosphere. After the addition, stirring was continued for additional 1 hour and the precipitated solid was filtered, washed with hexane and dried over solid NaOH for 3 hours. The dried powder showed no traces of impurity in ³¹P spectrum.

### Example 108

N-[N-(tert-butyloxycarbonyl)-3-aminopropyl]benzylamine (47).

A solution of N-(3-aminopropyl)benzylamine (38 g, 231.71 mmoles) in dry tetrahydrofuran (300 mL) was cooled to 5 C. in an ice-alcohol bath. To this cold stirred solution 2-[[(tert-butyoxycarbonyl)oxy]imino]-2-phenylacetonitrile (BOC-ON) (56.58 g, 230 mmoles) in dry tetrahydrofuran (300 mL) was added slowly during a 6 hour period. After the addition of BOC-ON, the reaction mixture was stirred at room temperature under argon for an additional 6 hours. The reaction mixture was evaporated to dryness and the residue was dissolved in ether (750 mL). The ether extract was washed with 5% sodium hydroxide solution (4×100 mL), dried over anhydrous sodium sulfate, and concentrated to dryness. The residue was purified by flash column using a chromatography over a silica dichloromethane: methanol gradient. The pure fractions were pooled together and evaporated to give 49.5 g (81%) of product as oil: ¹H nmr (deuteriochloroform): 1.42 (s, 9H, t-Boc), 1.65 (m, 2H, CH₂CH₂CH₂), 2.70 (t, 2H, CH₂NHCH₂), 3.20 (m, 2H, BocNHCH₂), 3.78 (s, 2H, ArCH₂), 5.32 (br s, 1H, BocNH), 7.30 (m, 5H, ArH).

### Example 109

10-Cyano-9-(phenylmethyl)-2,2-dimethyl-3-oxa-4-oxo-5,9-diazadecane (48).

To a stirred solution of the compound 47 (24 g, 91 mmoles) in dry acetonitrile (500 ml) was added potassium/celite (50 g) and chloroacetonitrile (27.3 g, 364 mmoles) at room temperature. The reaction mixture was placed in a preheated oil bath at 85° C. and allowed to stir at that temperature under argon for 12 hours. The reaction mixture was cooled, filtered and washed with dichloromethane (100 mL). The combined filtrate was evaporated to dryness. The residue was dissolved in dichloromethane (100 mL) and washed with 5% sodium bicarbonate solution (100 mL), water (100 mL) and brine (100 mL). The organic extract was dried over anhydrous sodium sulfate and concentrated to give a solid. The solid was crystallized from dichloromethane/hexane to give 24 g ((87%) as colorless needles, mp 70-73° C.; ¹H nmr (deuteriochloroform): 1.44 (s, 9H, t-Boc), 1.71 (m, 2H, CH₂CH₂CH₂), 2.67 (t, 2H, J=6.4Hz, CH₂NHCH₂), 3.23 (m, 2H,BocNHCH₂), 3.46 (s, 2H, CH₂CN), 3.65 (s, 2H, ArCH₂), 4.85 (br s, 1H, BocNH), 7.33 (s, 5H, ArH).

Anal. Calcd. for C₁₇H₂₅N₃O₂: C, 67.29; H, 8.31; N, 13.85, Found: C, 67.34; H, 8.45; N, 13.85.

### Example 110

9,12-Di(phenylmethyl)-2,2-dimethyl-3-oxa-4-oxo-5,9,12-triazadodecane (49).

The nitrile compound of Example 48 (34 g, 112.21 mmoles) was dissolved in ethanol (100 mL) and placed in a parr hydrogenation bottle. Sodium hydroxide (7 g) was dissolved in water (20 mL), mixed with ethanol (180 mL) and added into the parr bottle. Ra/Ni (5 g, wet) was added and shaked in a parr apparatus over hydrogen (45 psi) for 12 hours. The catalyst was filtered, washed with 95% ethanol (100 mL). The combined filtrate was concentrated to 100 mL and cooled to 5° C. in an ice bath mixture. The cold solution was extracted with dichloromethane (3×200 mL). The combined extract dried over anhydrous sodium sulfate and evaporated to give 32 g (92%) of an oil product. The product was used as such for the next reaction. ¹H nmr (deuteriochloroform): 1.32 (br s, 2H, NH₂), 1.42 (s, 9H, t-Boc), 1.67 (m, 2H, CH₂CH₂CH₂), 2.48 (m, 4H, CH₂CH₂NH₂), 2.75 (t, 2H, J=6.4Hz, CH₂NHCH₂), 3.15 (m, 2H, BocNHCH₂), 3.55 (s, 2H, ArCH₂), 5.48 (br s, 1H, BocNH), 7.31 (m, 5H, ArH).

The above amine (33 g, 107.5 mmoles) in dry methanol (100 mL) was mixed with anhydrous magnesium sulfate (30 g) and allowed to stir at room temperature under argon atmosphere. To this stirred solution benzaldehyde (13.2 g, 125 mmoles) was added and the stirring was continued for 4 hours under argon. The reaction mixture was diluted with methanol (150 mL) and cooled to −5° C. in an ice salt bath. Solid sodium borohydride (30 g) was added in 1 g lots at a time during 2 hour periods, keeping the reaction temperature below 0° C. After the addition of sodium borohydride, the reaction mixture was allowed to stir at room temperature overnight and filtered over celite. The filtrate was evaporated to dryness. The residue was partitioned between water (350 mL)/ether (500 mL) and extracted in ether. The ether extract was dried over anhydrous sodium sulfate and evaporated to dryness. The residue was purified on a silica gel column using dichloromethane:methanol as eluent. The pure fractions were pooled together and evaporated to give 35 g (82%) as oil; ¹H nmr (deuteriochloroform): 1.42 (s, 9H, t-Boc), 1.65 (m, 2H, CH₂CH₂CH₂), 1.75 (br s, 1H, ArCH₂NH), 2.55 (m, 4H,CH₂CH₂, 2.70 (t, 2H, J=6.4Hz, CH₂NHCH₂), 3.15 (m, 2H, BocNHCH₂), 3.52 (s, 2H, ArCH₂), 3.72 (s, 2H, ArCH₂), 5.55 (br s, 1H, BocNH), 7.28 (m, 10 H, ArH).

Anal. Calcd. for C₂₄H3₅N₃O₂: C, 72.51; H, 8.87; N, 10.57. Found: C, 72.39; H, 8.77; H, 10.72.

### Example 111

13-cyano-9,12-di(phenylmethyl)-2,2-dimethyl-3-oxa-4-oxo-5,9,12-triazatridecane (50).

The title compound was prepared from compound 49 by following the procedure used for the preparation of the compound of Example 48. Materials used: Substrate 49 (4.55 g, 11.46 mmoles); chloro acetonitrile (2.6 g, 34.38 mmoles); potassium fluoride/celite (9.0 g) and dry acetonitrile (100 mL). The crude product was purified by flash chromatography over silica gel using dichloromethane:acetone as the eluent to give 4.8 g (96%); ¹H nmr (deuteriochloroform): 1.42 (s, 9H, t-Boc), 1.68 (m, 2H, CH₂CH₂CH₂), 2.52 (m, 4H, CH₂CH₂), 2.68 (t, 2H, J=6.2Hz, CH₂NHCH₂), 3.22 (m, 2H, BocNHCH₂), 3.36 (s, 2H, CNCH₂), 3.50 (s, 2H, ArCH₂), 3.62 (s, 2H, ArCH₂), 5.72 (br s, 1H, BocNH), 7.32 (m, 10H, ArH).

Anal. Calcd. for C₂₆H₃₆H₄O₂: C, 71.52; H, 8.31; H, 12.83. Found: C, 71.17; H, 8.14; N, 12.82.

### Example 112

9,12,15-Tri(phenylmethyl)2,2-dimethyl-3-oxa-4-oxo-5,9,12,15-tetraazapentadecane (51).

The title compound was prepared from compound 50 by following a two step procedure used in Example 49. Materials used in the first step: The substrate 50 (25 g, 57.34 mmoles); Ra/Ni (5 g); sodium hydroxide in ethanol (200 mL, 7 g of sodium hydroxide was dissolved in 20 mL of water and mixed with ethanol) and ethanol used to dissolve the substrate (100 mL). The crude product was extracted in dichloromethane which on evaporation gave 22 g (87%) of an oily product; ¹H nmr (deuteriochloroform): 1.40 (s, 9H, t-Boc), 1.50 (m, 4H, CH₂CH₂CH₂ & NH₂), 2.48 (m, 8H, 2 CH₂CH₂), 2.66 (t, 2H, J=6.2Hz, CH₂NHCH₂), 3.24 (m, 2H, BocNHCH₂), 3.50 (s, 2H, ArCH₂), 3.56 (s, 2H, ArCH₂), 5.48 (br s, 1H, BocNH), 7.28 (m, 10H, ArH).

Materials used in the second step: Above amine (24.4 g, 55.33 mmoles); benzaldehyde (6.36 g, 60.00 mmoles); magnesium sulfate (20.0 g) and dry methanol (200 mL). The crude product was purified by flash chromatography over silica gel using dichloromethane:methanol as the eluent to give 20.0 g (68%) of compound 51 as oil; ¹H nmr (deuteriochloroform): 1.40 (s, 9H, t-Boc), 1.52 (m, 2H, CH₂CH₂CH₂), 1.84 (br s, 1H, ArCH₂NH), 2.38 (t, 2H, J=6.2Hz, CH₂NHCH₂), 2.54 (m, 8H 2 CH₂CH₂), 3.08 (m, 2H, BocNHCH₂), 3.42 (s, 2H, ArCH₂), 3.50 (s, 2H, ArCH₂), 3.65 (s, 2H, ArCH₂), 3.65 (s, 2H, ArCH₂), 5.45 (br s, 1H, BocNH), 7.28 (m, 15H, ArH).

Anal. Calcd. for C₃₃H₄₆N₄O₂: C, 74.67; H, 8.74; N, 10.56. Found: C, 74.92; H, 8.39; N, 10.71.

### Example 113

16-Cyano-9,12,15-tri(phenylmethyl)-2,2-dimethyl-3-oxa-oxo-5,9,12,15-tetraazahexadecane (52).

The title compound was prepared from compound 51 by following the procedure used in Example 48. Materials used: Substrate (Example 51 compound 51, 8.30 g, 15.66 mmoles); chloro acetonitrile (3.52 g, 46.98 mmoles); potassium fluoride/celite (10.0 g and dry acetonitrile (150 mL). The crude product was purified by flash chromatography over silica gel using dichloromethane:ethyl acetate as the eluent to give 7.6 g (85%); ¹H nmr (deuteriochloroform): 1.42 (s, 9H, t-Boc), 1.60 (m,2H, CH₂CH₂CH₂), 2.42 (t, 2H, J=6.2Hz, CH₂NHCH₂), 2.60 (m, 8H, 2CH₂CH₂), 3.14 (m, 2H, BocNHCH₂), 3.38 (s, 2H, CNCH₂), 3.48 (s, 2H, ArCH₂), 3.54 (s, 2H, ArCH₂), 3.60 (s, 2H, ArCH₂), 5.42 (br s, 1H, BocNH), 7.26 (m, 15H, ArH).

Anal. Calcd. for C₃₅H₄₇N₅O₂: C, 73,77; H, 8.32; N, 12.29. Found: C, 73.69; H, 8.19; N, 12.31.

### Example 114

9,12,15,18-Tetra(phenylmethyl)-2,2-dimethyl-3-oxa-4-oxo-5,9,12,15,18-petaazaoctadecane (53).

The title compound was prepared from compound 52 by following a two step procedure used for the preparation of the Example 49 compound 49. Materials used in the first step: The substrate (compound 52, 7 g, 12.30 mmoles); Ra/Ni (2 g); sodium hydroxide in ethanol (160 mL, 3.5 g of sodium hydroxide was dissolved in 10 mL of water and mixed with ethanol) and ethanol used to dissolve the substrate (100 ml). The crude product was extracted in dichloromethane which on evaporation gave 5.6 g (79%) as oil; ¹H nmr (deuteriochloroform): 1.40 (s, 9H, t-Boc), 1.50 (m, 4H, CH₂CH₂CH₂ & NH₂), 2.48 (m, 12H, 3 CH₂CH₂), 2.66 (m, 2H,CH₂NHCH₂), 3.24 (m, 2H, BocNHCH₂), 3.50 (s, 2H, ArCH₂), 3.56 (s, 4H, 2 ArCH₂), 3.62 (s, 2H, ArCH₂), 5.48 (br s, 1H, BocNH), 7.28 (m, 15H, ArH).

Material used in the second step: above amine (21.2 g, 36.74 mmoles); benzaldehyde (4.24 g, 40.00 mmoles); magnesium sulfate (10.0 g), dry methanol (200 mL) and sodium borohydride (4.85 g, 128.45 mmoles). The crude product was purified by flash chromatography over silica gel using dichloromethane:methanol as the eluent to give 18.67 g (77%) of compound 53 as oil; ¹H nmr (deuteriochloroform): 1.40 (s, 9H, t-Boc), 1.52 (m, 2H, CH₂CH₂CH₂), 2.05 (br s, 1H, ArCH₂NH), 2.38 (t, 2H, J=6.0Hz, CH₂NHCH₂), 2.54 (m, 12H, 2 CH₂CH₂), 3.08 (m, 2H, BocNHCH₂), 3.40 (s, 2H, ArCH₂), 3.50 (s, 4H, 2 ArCH₂), 3.64 (s, 2H, ArCH₂), 5.55 (br s, 1H, BocNH), 7.28 (m, 20H, ArH).

Anal. Calcd. for C₄₂H₅₇N₅O₂: C, 75.98; H, 8.65; N, 10.55. Found: C, 75.72; H, 8.67; N, 10.39.

### Example 115

13-amino-1,4,7,10-tetra(phenylmethyl)-1,4,7,10-tetraazatridecane (54).

To a stirred solution of compound 53 (2.65 g, 4 mmoles) in dichloromethane (10 mL) was added trifluoroacetic acid (10 mL) at room temperature. The reaction mixture was allowed to stir at room temperature for 30 minutes and evaporated to dryness. The residue was dissolved in dichloromethane (100 mL) and washed with 5% sodium bicarbonate solution (150 mL) to pH 8, and brine (50 mL). The organic extract was dried over anhydrous sodium sulfate and concentrated to dryness. The oily residue that obtained was used as such for the next reaction. ¹H nmr (deuteriochloroform): 1.50 (m, 5H, CH₂CH₂CH₂, NH₂, & ArCH₂NH), 2.38 (t, 2H, J=6.4Hz, CH₂NHCH₂), 2.54 (m, 14H, 7 CH₂), 3.52 (s, 2H, ArCH₂), 3.56 (s, 4H, 2 ArCH₂). 3.62 (s, 2H, ArCH₂), 7.28 (m, 20H, ArH).

### Example 116

3′,5′-O-(Tetraisopropyldisiloxane-1 3-diyl)-N-[4,7,10,13-tetrakis-(phenylmethyl)-4,7,10,13-tetraazatridec-1-yl]-2′-deoxyquanosine (56).

A mixture of 2-chloroinosine (55 in reaction scheme 3, 2.12 g, 4 mmoles) and compound 54 (2.5 g, 4.4 mmoles) in 2-methoxyethanol (50 mL) was heated at 80° C. for 12 hours. The reaction mixture was evaporated to dryness and the residue on flash chromatography over silica gel using dichloromethane and methanol (9:1) gave 2.55 g (60%) of the title compound as foam. ¹H nmr (deuteriochloroform): 1.00 (m, 24H, 4 Isobutyl-H), 1.62 (m, 1H, C_{2′}H), 1.80 (m, 4H, CH₂CH₂CH₂, C_{2′}H, & ArCH₂NH), 2.52 (m, 14H, 7 CH₂), 3.20 (s, 2H, ArCH₂), 3.32 (s, 2H, ArCH₂), 3.42 (s, 2H, ArCH₂), 3.48 (s, 4H, ArCH₂ & CH₂), 3.78 (m, 1H, C_{4′}H), 4.05 (m, 2H, C_{5′}CH₂), 4.72 (m, 1H, C_{3′}H), 6.22 (m, 1H, C_{1′}H), 6.94 (m. 1H, N₂H), 7.26 (m, 20H, ArH), 7.72 (s, 1H, C₈H), 10.52 (br s, 1H, NH).

Anal. Calcd. for C₅₉H₈₅N₉O₅Si₂: C, 67.07; H, 8.11; N, 11.93. Found: C, 67.22; H, 8.24; N, 11.81.

### Example 117

3′,5′-O-(Tetraisopropyldisiloxane-1,3-diyl)-6-O-(phenylmethyl)-N-[15-methyl-14-oxo-4,7,10,13-tetrakis (phenylmethyl)-4,7,10,13-tetraazahexadec-1-yl]-2′-deoxyguanosine (57).

The compound of Example 55 (2.00 g, 1.89 mmoles) was coevaporated with dry pyridine (30 mL) two times. The resulting residue was dissolved in dry pyridine (50 mL) and cooled to 0° C. in an ice bath mixture. To this cold stirred solution was added triethylamine (0.61 g, 6 mmoles) followed by isobutyryl chloride (0.64 g, 6 mmoles) slowly under argon atmosphere. After the addition of isobutyryl chloride, the reaction mixture was stirred at room temperature for 12 hours and evaporated to dryness. The residue was dissolved in dichloromethane (150 mL), washed with 5% sodium bicarbonate (50 mL), water (50 mL) and brine (50 mL). The organic extract was dried over anhydrous sodium sulfate and evaporated to dryness. The residue on purification over silica gel using dichloromethane/methanol (95:5) gave 1.88 g (88%) of the title compound as a foam.

The above foam (1.8 g, 1.61 mmoles) was dried over phosphorous pentaoxide under vacuum for 12 hours. The dried residue was dissolved in dry dioxane (50 mL) and treated with triphenyl phosphine (0.83 g, 3.2 mmoles), benzyl alcohol (0.35 g, 3.2 mmoles), and diethylazodicarboxylate (0.54 g, 3.2 mmoles) at room temperature under argon atmosphere. The reaction mixture after stirring for 10 hours evaporated to dryness. The residue was dissolved in dichloromethane (150 mL) and washed with 5% sodium bicarbonate (50 mL), water (50 mL) and brine (50 mL). The organic extract was dried over anhydrous sodium sulfate and evaporated to dryness. The residue was flash chromatographed over silica gel using dichloromethane/acetone (7:3) as the eluent. The pure fractions were collected together and evaporated to give 1.7 g (74%) of foam: ¹H nmr (deuteriochloroform) : 1.04 (m, 30H, 5 Isobutyl-CH₃), 1.68 (m, 2H, CH₂CH₂CH₂), 2.55 (m, 16H, 7 CH₂, C_{2′}H, & isobutyl-CH), 3.08 (m, 1H, C_{2′}H), 3.36 (m, 2H, CH₂), 3.52 (m, 8H, 4 ArCH₂), 3.84 (m, 1H, C_{4′}H), 4.00 (m, 2H, C_{5′}CH₂), 4.72 (m, 1H, C_{3′}H), 5.50 (s, 2H, ArCH₂), 6.18 (m, 1H, C_{1′}H) 7.04 (m, 1H, N₂H), 7.26 (m, 25H, ArH), 7.76 (s, 1H, C₈H)

Anal. Calcd. for C₇₀H₉₇N₉O₆Si₂: C, 69.09; H, 8.04; N, 10.36. Found: C, 69.12; H, 8.23; N, 10.19.

### Example 118

6-O-(Phenylmethyl)-N-[15-methyl-14-oxo-4,7,10,13-tetrakis(phenylmethyl)-4,7,10,13-tetraazahexadec-1-yl]-2′-deoxyguanosine (58).

To a stirred solution of compound 57 (5.0 g, 4.11 mmoles) in pyridine (50 mL) was added freshly prepared 1N solution of tetrabutylammonium fluoride (20 mL, 20 mmoles; prepared in a mixture of pyridine:tetrahydrofuran:water in the ratio of 5:4:1) at room temperature. The reaction mixture was allowed to stir for 30 minutes and quenched with H⁺ resin (pyridinium form) to pH 6-7. The resin was filtered, washed with methanol (50 mL), and the combined filtrate evaporated to dryness. The residue was dissolved in dichloromethane (200 mL), washed with water (50 mL), and brine (50 mL). The organic extract was dried over sodium sulfate and concentrated to dryness. The foam that obtained was purified by flash chromatography over silica gel column using dichloromethane/methanol (95:5) as the eluent. The required fractions were collected together and evaporated to give 3.5 g (87%) of the titled compound as foam. ¹H nmr (deuteriochloroform): 1.04 (m, 30H, 5 isobutyryl CH₃), 1.68 (m, 2H, CH₂CH₂CH₂), 2.55 (m, 16H, 7 CH₂, C_{2′}H, & isobutyryl CH), 3.08 (m, 1H, C_{2′}H), 3.36 (m, 2H, CH₂), 3.52 (m, 8H, 4 ArCH₂), 3.84 (m, 1H, C_{4′}H), 4.00 (m, 2H, C_{5′}CH₂), 4.72 (m, 1H, C_{3′}H), 5.50 (s, 2H, ArCH₂), 6.18 (m, 1H, C_{1′}H), 7.04 (m, 1H, N₂H), 7.26 (m, 25H, ArH), 7.76 (s, 1H, C₈H).

Anal. Calcd. for C₇₀H₉₇N₉O₆Si₂: C, 69.09; H, 8.04; N, 10.36. Found: C, 69.12; H, 8.23; N, 10.19.

What is claimed is:

1. An antisense compound 8 to 30 nucleobases in length targeted to the 5′ untranslated region, coding region, intron:exon junction, intron region, exon region, translation termination codon region or 3′ untranslated region of a nucleic acid molecule encoding mdm2, wherein said antisense compound modulates the expression of mdm2.

2. The antisense compound of claim 1 wherein said antisense compound inhibits the expression of human mdm2.

3. The antisense compound of claim 1 which is an antisense oligonucleotide.

4. An antisense compound up to 30 nucleobases in length comprising at least an 8-nucleobase portion of SEQ ID NO: 3, 4, 5, 7, 10, 15, 17, 18, 19, 21, 36, 42, 52, 54, 59, 60, 61, 62, 64, 66, 67, 68, 69, 70, 72, 73, 74, 75, 77, 78, 80, 81, 84, 88, 90, 96, 98, 103, 105, 109, 111, 114, 117, 118, 120, 121, 124, 126, 127, 129, 130, 137, 145, 147, 151, 154, 156, 158, 160, 165, 171, 175, 177, 178, 180, 182, 183, 184, 186, 188, 189, 191, 192, 193, 195, 196, 197, 199, 200, 201, 203, 206, 210, 212, 215, 216, 218, 221, 225, 231, 235, 241, 243, 245, 246, 249, 251, 254, 256, 258, 260, 264, 268, or 373 which inhibits the expression of mdm2.

5. The antisense compound of claim 2 which is targeted to the 5′ untranslated region of the S-mdm2 transcript.

6. The antisense compound of claim 1 which contains at least one phosphorothioate intersugar linkage.

7. The antisense compound of claim 1 which has at least one 2′-O-methoxyethyl modification.

8. The antisense compound of claim 1 which contains at least one 5-methyl cytidine.

9. The antisense compound of claim 8 in which every 2′-O-methoxyethyl modified cytidine residue is a 5-methyl cytidine.

10. A pharmaceutical composition comprising the antisense compound of claim 1 and a pharmaceutically acceptable carrier or diluent.

11. The pharmaceutical composition of claim 10 wherein said pharmaceutically acceptable carrier or diluent further comprises a lipid or liposome.

12. A method of modulating the expression of mdm2 in cells or tissues comprising contacting said cells or tissues with the antisense compound of claim 1.

13. A method of reducing hyperproliferation of human cells comprising contacting proliferating human cells with the antisense compound of claim 2 or a pharmaceutical composition comprising said antisense compound.

14. A method of treating an animal having a disease or condition associated with mdm2 comprising administering to said animal a therapeutically or prophylactically effective amount of an antisense compound of claim 1.

15. The method of claim 14 wherein the disease or condition is associated with overexpression of mdm2 and the antisense compound inhibits the expression of mdm2.

16. The method of claim 14 wherein the disease or condition is associated with amplification of the mdm2 gene and the antisense compound inhibits the expression of mdm2.

17. The method of claim 14 wherein the disease or condition is a hyperproliferative condition and the antisense compound inhibits the expression of mdm2.

18. The method of claim 17 wherein the hyperproliferative condition is cancer.

19. The method of claim 18 wherein the cancer is a blood, bone, brain, breast, lung or a soft tissue cancer.

20. The method of claim 17 wherein the hyperproliferative condition is psoriasis, fibrosis, atherosclerosis or restenosis.

21. The method of claim 14 wherein said antisense compound is administered in combination with a chemotherapeutic agent to overcome drug resistance.

22. An antisense compound up to 30 nucleobases in length targeted to the translational start site of a nucleic acid molecule encoding human mdm2, wherein said antisense compound inhibits the expression of said human mdm2 and comprises at least an 8-nucleobase portion of SEQ ID NO: 56, SEQ ID NO: 57, SEQ ID NO: 60, SEQ ID NO: 61, SEQ ID NO: 69, SEQ ID NO: 70 or SEQ ID NO: 72.

23. The antisense compound of claim 22 which contains at least one phosphorothioate intersugar linkage.

24. The antisense compound of claim 22 which has at least one 2′-O-methoxyethyl modification.

25. The antisense compound of claim 22 which contains at least one 5-methyl cytidine.

26. The antisense compound of claim 25 in which every 2′-O-methoxyethyl modified cytidine residue is a 5-methyl cytidine.

27. A pharmaceutical composition comprising the antisense compound of claim 22 and a pharmaceutically acceptable carrier or diluent.

28. The pharmaceutical composition of claim 27 wherein said pharmaceutically acceptable carrier or diluent comprises a lipid or liposome.

29. A method of modulating the expression of human mdm2 in cells or tissues comprising contacting said cells or tissues with the antisense compound of claim 22.

30. A method of reducing hyperproliferation of human cells comprising contacting proliferating human cells with the antisense compound of claim 22.

31. A method of reducing hyperproliferation of human cells comprising contacting proliferating human cells with the pharmaceutical composition of claim 27.

32. A method of treating an animal having a disease or condition associated with mdm2 comprising administering to said animal a therapeutically or prophylactically effective amount of the antisense compound of claim 22.

33. The method of claim 32 wherein the disease or condition is associated with overexpression of mdm2 and the antisense compound inhibits the expression of mdm2.

34. The method of claim 32 wherein the disease or condition is associated with amplification of the mdm2 gene and the antisense compound inhibits the expression of mdm2.

35. The method of claim 32 wherein the disease or condition is a hyperproliferative condition and the antisense compound inhibits the expression of mdm2.

36. The method of claim 35 wherein the hyperproliferative condition is cancer.

37. The method of claim 36 wherein the cancer is a blood, bone, brain, breast, lung or a soft tissue cancer.

38. The method of claim 35 wherein the hyperproliferative condition is psoriasis, fibrosis, atherosclerosis or restenosis.

39. The method of claim 32 wherein said antisense compound is administered in combination with a chemotherapeutic agent to overcome drug resistance.

40. A method of modulating apoptosis in cells or tissues comprising contacting said cells or tissues with the compound of claim 1 so that apoptosis is modulated.

41. A method of modulating apoptosis in cells or tissues comprising contacting said cells or tissues with the compound of claim 22 so that apoptosis is modulated.

42. A method of inducing the expression of p21 in cells or tissues comprising contacting said cell with the compound of claim 1 so that p21 expression is increased.

43. A method of inducing the expression of p21 in cells or tissues comprising contacting said cells or tissues with the compound of claim 22 so that p21 expression is increased.

44. An oligonucleotide comprising at least one nucleotide comprising a heterocycle member covalently bound to a substituted sugar member which is further covalently bound through at least one linker to a sugar moiety member of a second nucleotide, said at least one modified nucleotide described according to structure I; 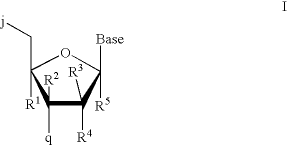
j and q are each independently covalently linkers of about 1-15 atoms selected from the group comprising phosphorothioates, methylene(methylimino),phosphodiester, morpholino, amide, thioamide, polyamide, (CH₂)ₙ(G)N(R¹¹) (G)N(R¹¹), (CH₂)ₙN (G) R¹¹, N— (CH₂)ₙ(G) R¹¹ and (CH₂)ₙN(R¹¹)C(G)where G is a heteroatom, n is an integer between about 0 and 5 and each R¹¹ is independently selected from the group comprising alkyl, heteroalkyl, cyclic alkyl, heterocycle, aryl, heteroaryl and hydrogen;
R¹, R², R³, R⁴ and R⁵ are each independently selected from the group comprising halo, hydrogen and GR¹¹ and;
where Base is a nucleobase selected from the group comprising structure II, structure III or structure IV; 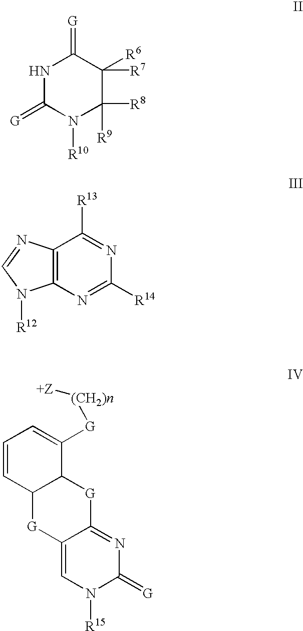
where R⁶, R⁷, R⁸, R⁹, R¹⁰, R¹¹, R¹², R¹³, R¹⁴ and R¹⁵ are independently selected from members of the group comprising alkyl, heteroalkyl, cyclic alkyl, heterocycle, aryl, heteroaryl, halo and hydrogen, and;
where G is a heteroatom and Z+ is a hypervalent species selected from the group comprising a quaternary amine, a cationic alkyl oxygen member, an alkyl sulfonium member or an alkyl phosphonium member and;
where at least one of R¹, R², R³, R⁴, R⁵, R¹⁰, R⁹, R⁸, R⁷, R⁶, R¹², R¹³, R¹⁴ or R¹⁵ is substituted, forming thereby said modified nucleotide.

45. The oligonucleotide according to claim 44 wherein G is O and R⁴ is 2′-O-dimethylamine oxyethylene and the Base is according to structure II;
Wherein R¹⁰ is a bond to the sugar, j is O, q is 3′-O-(2-methoxyethyl).

46. The oligonucleotide according to claim 44 wherein said nucleotide is according to structure IV;

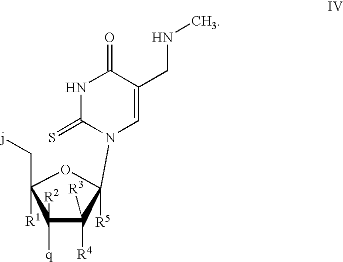

47. The oligonucleotide according to claim 44 where G is oxygen and R¹¹ is selected from the group comprising;

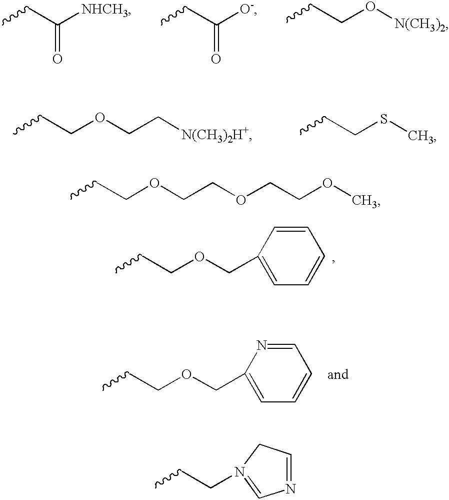

48. The oligonucleotide according to claim 44 wherein the Base is according to structure V; 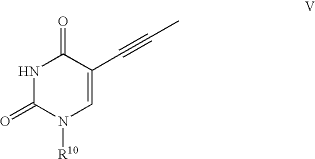
where R¹⁰ is a bond to the sugar.

49. The oligonucleotide according to claim 44 further associated with a pharmaceutically acceptable carrier, diluent, prodrug or lubricant.

50. The oligonucleotide according to claim 44 which is targeted to a nucleic acid encoding mdm2.
